SEQUENCE LISTING (37 sequences)

SEQ ID NO: 1 - DNA, 62 bp, Artificial Sequence
  Template UEI-DraIII
gagcggataacaatttcacacaggcacggggtgtgagatcacagatcggaagagcgtcgtgt

SEQ ID NO: 2 - DNA, 24 bp, Artificial Sequence
  UEI-Fwd
gagcggataacaatttcacacagg

SEQ ID NO: 3 - DNA, 19 bp, Artificial Sequence
  UEI-Rev
acacgacgctcttccgatc

SEQ ID NO: 4 - DNA, 142 bp, Artificial Sequence
  Template Calibrator-N15-AlwNI
  misc_feature (86)..(110): n is a, c, g, or t
tcgtcggcagcgtcagatgtgtataagagacagggaagacgtagcaagggagtagccaatgtgaattgagagccttaagctgtatnnnnnnnnnnnnnnnnnnnnnnnnncaggggctggtcgtgactgggaaaaccctggc

SEQ ID NO: 5 - DNA, 23 bp, Artificial Sequence
  Calib-Rev
gccagggttttcccagtcacgac

SEQ ID NO: 6 - DNA, 19 bp, Artificial Sequence
  Illu-2-Fwd
tcgtcggcagcgtcagatg

SEQ ID NO: 7 - DNA, 67 bp, Artificial Sequence
  Template UEI-N15-DraIII*
  misc_feature (34)..(48): n is a, c, g, or t
gagcggataacaatttcacacaggcacggggtgnnnnnnnnnnnnnnngatcggaagagcgtcgtgt

SEQ ID NO: 8 - DNA, 149 bp, Artificial Sequence
  Template Calibrator-N15-AlwNI-lg
  misc_feature (103)..(117): n is a, c, g, or t
tcgtcggcagcgtcagatgtgtataagagacagggaagacgtagcaagggagtagccaatgtgaattgagagccttaagctgtatcaggaccagagagatgannnnnnnnnnnnnnncaggggctggtcgtgactgggaaaaccctggc

SEQ ID NO: 9 - DNA, 53 bp, Artificial Sequence
  Illu1 rev UEI
gtctcgtgggctcggagatgtgtataagagacagacacgacgctcttccgatc

SEQ ID NO: 10 - DNA, 19 bp, Artificial Sequence
  Illu1
gtctcgtgggctcggagat

SEQ ID NO: 11 - DNA, 90 bp, Artificial Sequence
  Template-AlwNI-N15-Calib-DraIII
  misc_feature (44)..(58): n is a, c, g, or t
ggaagacgtagcaagggagtagccaatgtgaattcacacagtgnnnnnnnnnnnnnnncaggggctggtcgtgactgggaaaaccctggc

SEQ ID NO: 12 - DNA, 24 bp, Artificial Sequence
  Calib-Fwd
ggaagacgtagcaagggagtagcc

SEQ ID NO: 13 - DNA, 80 bp, Artificial Sequence
  RTmimicks
  misc_feature (36)..(43): n is a, c, g, or t
ctgcgcggatcccggaagcgaggccagctggctgcnnnnnnnnttggctgtctcttatacacatctgacgctgccgacga

SEQ ID NO: 14 - DNA, 38 bp, Artificial Sequence
  antiSBACA-AlwN1
gcagccagctggcctcgcttccgggatccgcgcagaca

SEQ ID NO: 15 - DNA, 101 bp, Artificial Sequence
  Template UEI-N15-DraIII-Illu1
  misc_feature (34)..(48): n is a, c, g, or t
gagcggataacaatttcacacaggcacggggtgnnnnnnnnnnnnnnngatcggaagagcgtcgtgtctgtctcttatacacatctccgagcccacgagac

SEQ ID NO: 16 - RNA, 539 nt, Staphylococcus aureus
auuaauacgacucacuauaccuagaucacagagaugugauggaaaauaguugaugaguuguuuaauuuuaagaauuuuuaucuuaauuaaggaaggagugauuucaauggcacaagauaucauuucaacaaucggugacuuaguaaaauggauuaucgacacagugaacaaauucacuaaaaaauaagaugaauaauuaauuacuuucauuguaaauuuguuaucuacguauaguacuaaaaguaugaguuauuaagccaucccaacuuaauaaccauguaaaauuagcaagugaguaacauuugcuaguagaguuaguuuccuuggacucagugcuauguauuuuucuuaauuaucauuacagauaauuauuucuagcauguaagcuaucguaaacaacaucgauuuaucauuauuugauaaauaaaauuuuuuucauaauuaauaacauccccaaaaauagauugaaaaaauaacuguaaaacauucccuuaauaauaaguauggucgugagccccucccaagcucgcggccuuuug

SEQ ID NO: 17 - DNA, 10 bp, Artificial Sequence
  empty
  misc_feature (1)..(10): n is a, c, g, or t
nnnnnnnnnn

SEQ ID NO: 18 - DNA, 55 bp, Artificial Sequence
  RT-UMI-RNAIII*
  misc_feature (36)..(41): n is a, c, g, or t
ctgcgcggatcccggaagcgaggccagctggctgcnnnnnnttgggaggggctca

SEQ ID NO: 19 - DNA, 10 bp, Artificial Sequence
  empty
  misc_feature (1)..(10): n is a, c, g, or t
nnnnnnnnnn

SEQ ID NO: 20 - DNA, 35 bp, Artificial Sequence
  SB-alone
ctgcgcggatcccggaagcgaggccagctggctgc

SEQ ID NO: 21 - DNA, 63 bp, Artificial Sequence
  RNAIII-Fwd
tcgtcggcagcgtcagatgtgtataagagacagataactgtaaaacattcccttaataataag

SEQ ID NO: 22 - RNA, 867 nt, Artificial Sequence
  Gfp mRNA
augaccagcuacccauacgauguuccagauuacgcuaucgaaggccgcggccgccaucaucaucaucaucaugauaucgguaccaguaaaggagaagaacuuuucacuggaguugucccaauucuuguugaauuagauggugauguuaaugggcacaaauuuucugucaguggagagggugaaggugaugcaacauacggaaaacuuacccuuaaauuuauuugcacuacuggaaaacuaccuguuccauggccaacacuugucacuacuuucgcguauggucuucaaugcuuugcgagauacccagaucauaugaaacagcaugacuuuuucaagagugccaugcccgaagguuauguacaggaaagaacuauauuuuucaaagaugacgggaacuacaagacacgugcugaagucaaguuugaaggugauacccuuguuaauagaaucgaguuaaaagguauugauuuuaaagaagauggaaacauucuuggacacaaauuggaauacaacuauaacucacacaauguauacaucauggcagacaaacaaaagaauggaaucaaaguuaacuucaaaauuagacacaacauugaagauggaagcguucaacuagcagaccauuaucaacaaaauacuccaauuggcgauggcccuguccuuuuaccagacaaccauuaccuguccacacaaucugcccuuucgaaagaucccaacgaaaagagagaccacaugguccuucuugaguuuguaacagcugcugggauuacacauggcauggaugaacuauacaaagagaauucagagcucggauccacucgagaugcauuagaacaaaaauuauuaucagaagaagauuuaaauuaa

SEQ ID NO: 23 - DNA, 69 bp, Artificial Sequence
  gfp-mut2-RT-UMI-AlwNI*
  misc_feature (36)..(43): n is a, c, g, or t
ctgcgcggatcccggaagcgaggccagctggctgcnnnnnnnncaaataaatttaagggtaagttttcc

SEQ ID NO: 24 - DNA, 26 bp, Artificial Sequence
  Gfp-Fwd-25nt
tgaattagatggtgatgttaatgggc

SEQ ID NO: 25 - DNA, 70 bp, Artificial Sequence
  Template-UEI-DraIII-N2x5
  misc_feature (34)..(35): n is a, c, g, or t
  misc_feature (38)..(39): n is a, c, g, or t
  misc_feature (42)..(43): n is a, c, g, or t
  misc_feature (46)..(47): n is a, c, g, or t
  misc_feature (50)..(51): n is a, c, g, or t
gagcggataacaatttcacacaggcacggggtgnnacnngannctnngcnngatcggaagagcgtcgtgt

SEQ ID NO: 26 - DNA, 76 bp, Artificial Sequence
  Template-UEI-DraIII-N4443
  misc_feature (34)..(37): n is a, c, g, or t
  misc_feature (41)..(44): n is a, c, g, or t
  misc_feature (48)..(51): n is a, c, g, or t
  misc_feature (55)..(57): n is a, c, g, or t
gagcggataacaatttcacacaggcacggggtgnnnnacannnngagnnnntctnnngatcggaagagcgtcgtgt

SEQ ID NO: 27 - DNA, 85 bp, Artificial Sequence
  Template-Calibrator-AlwNI
ggaagacgtagcaagggagtagccaatgtgaattcacacagtgtctacaagtacaggggctggtcgtgactgggaaaaccctggc

SEQ ID NO: 28 - DNA, 93 bp, Artificial Sequence
  Template-Calibrator-AlwNI-N2x5
  misc_feature (44)..(45): n is a, c, g, or t
  misc_feature (48)..(49): n is a, c, g, or t
  misc_feature (52)..(53): n is a, c, g, or t
  misc_feature (56)..(57): n is a, c, g, or t
  misc_feature (60)..(61): n is a, c, g, or t
ggaagacgtagcaagggagtagccaatgtgaattcacacagtgnntcnntanncannagnncaggggctggtcgtgactgggaaaaccctggc

SEQ ID NO: 29 - DNA, 99 bp, Artificial Sequence
  Template-Calibrator-AlwNI-N4443
  misc_feature (44)..(47): n is a, c, g, or t
  misc_feature (51)..(54): n is a, c, g, or t
  misc_feature (58)..(61): n is a, c, g, or t
  misc_feature (65)..(67): n is a, c, g, or t
ggaagacgtagcaagggagtagccaatgtgaattcacacagtgnnnntctnnnnacannnngagnnncaggggctggtcgtgactgggaaaaccctggc

SEQ ID NO: 30 - DNA, 90 bp, Artificial Sequence; identical to SEQ ID NO: 11
  Template-Calibrator-AlwNI-N15
  misc_feature (44)..(58): n is a, c, g, or t

SEQ ID NO: 31 - DNA, 100 bp, Artificial Sequence
  Tag-NaBAb-AlwNI
  misc_feature (32)..(39): n is a, c, g, or t
cacacaggaaacagctatgacccagtgtctgnnnnnnnntgtggacgctgtctcttatacacatctgacgctgccgacgacgtcgtgactgggaaaaccc

SEQ ID NO: 32 - DNA, 20 bp, Artificial Sequence
  BG-M13-Fwd**
gggttttcccagtcacgacg

SEQ ID NO: 33 - DNA, 22 bp, Artificial Sequence
  Alexa488-M13-Rev
cacacaggaaacagctatgacc

SEQ ID NO: 34 - DNA, 38 bp, Artificial Sequence
  DNA based probe specific to RNAIII of Staphylococcus aureus
gcagccagctggcctcgcttccgggatccgcgcagggg

SEQ ID NO: 35 - DNA, 55 bp, Artificial Sequence
  DNA based probe specific to RNAIII of Staphylococcus aureus
  misc_feature (36)..(41): n is a, c, g, or t
gatcgcggatcccggaagcgaggccagctggctgcnnnnnnttgggaggggctca

SEQ ID NO: 36 - DNA, 62 bp, Artificial Sequence
  Example of entity identification sequence
  misc_feature (34)..(43): n is a, c, g, or t
gagcggataacaatttcacacaggcacggggtgnnnnnnnnnngatcggaagagcgtcgtgt

SEQ ID NO: 37 - DNA, 62 bp, Artificial Sequence
  example of entity identification sequence
  misc_feature (20)..(29): n is a, c, g, or t
acacgacgctcttccgatcnnnnnnnnnncaccccgtgcctgtgtgaaattgttatccgctc

(19) United States
(12) Patent Application Publication
(10) Pub. No.: US 2019/0316180 A1
(43) Pub. Date: Oct. 17, 2019

(54) TANDEM BARCODING OF TARGET MOLECULES FOR THEIR ABSOLUTE QUANTIFICATION AT SINGLE-ENTITY RESOLUTION

(71) Applicants: UNIVERSITE DE STRASBOURG, STRASBOURG (FR); CENTRE NATIONAL DE LA RECHERCHE SCIENTIFIQUE, PARIS (FR)

(72) Inventors: MICHAËL RYCKELYNCK, STRASBOURG (FR); STÉPHANIE BAUDREY, LINGOLSHEIM (FR)

(21) Appl. No.: 16/463,492

(22) PCT Filed: Nov. 23, 2017
(86) PCT No.: PCT/EP2017/080264

§ 371 Date: May 23, 2019

(30) Foreign Application Priority Data
Nov. 23, 2016 (EP) 16306543.6

(51) Int. Cl. C12Q 1/6806; B01L 3/00

(52) U.S. Cl. CPC C12Q 1/6806; B01L 3/50273

(57) ABSTRACT

The present invention relates to a new method of labelling any target molecules from a plurality of entities, preferably in high throughput regimes, i.e. allowing the analysis of several thousands of entities per run, while preserving the integrity of the single-entity information. This method is based on a tandem molecular barcoding in which all molecular targets are labelled with a first unique barcode which is different for each molecular target from an entity, and with a tag sequence coding the entity from which the molecular target originates. Once this tandem barcoding is performed, the absolute quantification of all molecular targets with a single-entity resolution may be carried out in a single run of next-generation sequencing. The present invention also relates to a method of quantifying one or several molecular targets from a plurality of entities with single-entity resolution, as well as a kit and the use of such kit to label a plurality of molecular targets from a plurality of entities according to the method of the invention.

### FIELD OF THE INVENTION

The present invention relates to methods and systems for labelling nucleic acids and other biological molecules from entities, e.g. cells, within emulsion droplets in high throughput regimes while preserving the integrity of the single-entity information.

### BACKGROUND OF THE INVENTION

Single cell analytics are gaining popularity due to the insight that taking into account the heterogeneity of a population of cell may be of capital interest to understand the function and behaviour of diverse biological systems. Individual cells of a population of clonal unicellular organisms or of multicellular organisms, even among cells of the same tissue or cell-type, often exhibit heterogeneous gene regulation or protein expression patterns. It is thus increasingly recognized that traditional analytical approaches that analyse large populations of cells yield ensemble views that, although informative, only reflect the dominant biological mechanism and fail to identify cell-to-cell variations.

RNA levels are considered a useful marker of phenotypic heterogeneity and, as a consequence, considerable efforts were done to analyse RNA content in single cells. Probe-dependent methods including fluorescence in situ hybridization (FISH) or reporter fusions to fluorescent proteins, was replaced with the probe-independent RNA-seq technique in which cellular RNA molecules are converted into cDNA and subsequently sequenced in parallel using next-generation sequencing technology. Single-cell RNA-seq requires the isolation of individual cells, the conversion of cellular RNA into cDNA and the massively parallel sequencing of cDNA libraries.

The rapid expansion of microfluidic devices has resulted in the development of valve-based microfluidic chips wherein cells are isolated in nano-liter reaction chambers (Streets et al., 2014). However, this approach remains limited not only by the cost but also because the number of single cells that can be currently processed with said chips remains at less than one hundred per run. Alternatively, microfluidic droplets also provide a compartment in which cells can be isolated. Typically, droplets of one phase are generated in another, immiscible phase by exploiting capillary instabilities in a microfluidic two-phase flow. The addition of a surfactant to either or both of the phases stabilizes the droplets against coalescence and allows them to function as discrete microreactors.

Furthermore, as single-cell RNA-seq analysis may require the profiling of several thousands if not millions of representative individual cells, barcoding strategies have been developed to reduce sequencing costs and increase throughput. Using unique cellular identifiers, it has made possible to pool up a multitude of cells for simultaneous sequencing since each read could subsequently be assigned to its original cell through the unique cellular barcode (Islam et al., 2012).

The main technical challenge when combining barcoding strategy and compartmentalization of cells into droplets is to ensure that each droplet carries a different barcode and thus that the integrity of the single cell information is preserved.

Several methods have been described to address this problem. Each cell may be co-encapsulated with a distinctly barcoded particle, such as bead (Macosko et al. 2015) or hydrogel microsphere (Klein et al., 2015), in a nano-liter scale droplet. Each of these particles contains more than 10⁸ individual primers that share the same “cell barcode”. In such a method, in order to ensure that a droplet comprises only one particle, the number of droplets created greatly exceeds the number of particles or cells injected, so that a droplet will generally contain zero or one cell and zero or one particle (Macosko et al. 2015; Klein et al., 2015).

Alternatively, a barcode-library emulsion may be produced using a microfluidic device consisting of 96 drop-makers creating millions of drops containing a high concentration of a single one of the 96 barcodes (Rotem et al. 2015). Each cell-bearing drop is then paired and fused with one barcode-drop. However, to ensure that each cell-bearing drop is fused with at most one barcode drop, only half of the cell-bearing drops actually fuse with a barcode drop. Furthermore, cases where two cell-bearing drops fuse with a single barcode drop or where two barcode drops fuse with a single cell-bearing drop, introduce errors in the resultant labelling and are a potential source of noise (Rotem et al. 2015).

Thus, whatever the method used to ensure that each droplet carries a different barcode, this results in a limited droplet occupancy, a reduced useful fraction of droplets, and thus a reduced throughput.

Furthermore, even if RNA levels have been recognized as useful marker for phenotypic heterogeneity, current methods provide limited information since levels of protein or other biological molecules cannot be assessed with the same system. Indeed, to date, quantification of the protein expression at the single cell level, which is critical for complete characterization of the phenotypic states, is generally based on fluorescence imaging methods.

Consequently, there is a great need for new methods and devices allowing high-throughput quantification of RNA, proteins and other biological molecules of interest at the single entity level.

### SUMMARY OF THE INVENTION

The present invention provides a new method of labelling any target molecules from a plurality of entities in high throughput regimes while preserving the integrity of the single-entity information.

Accordingly, in a first aspect, the present invention relates to a method of labelling a plurality of molecular targets from a plurality of entities while preserving the integrity of the single-entity information, said method comprising providing a first set of emulsion droplets comprising droplets containing labelled molecular targets, wherein each of these droplets contains a plurality of molecular targets originating from no more than one entity and wherein, in each of these droplets, each molecular target is labelled with a molecular identification DNA sequence comprising (i) a unique molecular identification (UMI) barcode which is different for each molecular target and (ii) an overhang or an overhang producing restriction site;

providing a second set of emulsion droplets comprising droplets containing entity identification sequences, wherein each of these droplets contains at least one entity identification sequence which is a DNA sequence, preferably a double stranded DNA sequence, comprising a unique entity identification (UEI) barcode which is different for each droplet of the second set, and an overhang producing restriction site;

fusing droplets of the first set with droplets of the second set wherein a droplet of the first set is fused with no more than one droplet of the second set; and

ligating UEI barcodes to labelled molecular targets, optionally after restriction enzyme digestion, and

optionally breaking the emulsion.

The method may further comprise

encapsulating a plurality of entities within emulsion droplets, each droplet containing no more than one entity, and optionally lysing said entities within the droplets to release molecular targets;

labelling said molecular targets with probes, each probe comprising

a capture moiety capable of specific binding or ligation to a molecular target or to an adaptor linked to said molecular target, and

a DNA moiety comprising (i) a region proximal to the capture moiety and comprising the unique molecular identification (UMI) sequence and (ii) a region distal from the capture moiety and comprising an overhang or an overhang producing restriction site,

thereby obtaining the first set of emulsion droplets.

The method may further comprise

(i) encapsulating a plurality of entity identification sequences within emulsion droplets, each droplet containing no more than one entity identification sequence, with an amplification reaction mixture, and

amplifying the entity identification sequences within droplets

thereby obtaining the second set of emulsion droplets, or

(ii) encapsulating a plurality of entity identification sequences within emulsion droplets in the presence of UEI-calibrators, wherein at least some droplets comprise one or several entity identification sequences and one or several UEI-calibrators, and wherein said UEI-calibrators are DNA sequences, preferably double stranded DNA sequences, comprising a unique calibrator barcode which is different for each UEI-calibrator and for each droplet, and one or two overhang producing restriction sites; and

amplifying entity identification sequences and/or UEI-calibrators within droplets;

thereby obtaining the second set of emulsion droplets.

Preferably, after fusion of droplets of the first and second sets, (i) UEI calibrator barcodes and UEI barcodes and (ii) UEI barcodes and labelled molecular targets are assembled through restriction enzyme digestion and ligation of compatible overhangs of (i) UEI calibrators and entity identification sequences and of (ii) entity identification sequences and labelled molecular targets.

Alternatively, after fusion of droplets of the first and second sets, (i) UEI calibrator barcodes, UEI barcodes and labelled molecular targets may be assembled through restriction enzyme digestion and ligation of compatible overhangs of (i) UEI calibrators and labelled molecular targets and (ii) UEI calibrators and entity identification sequences, or of i) UEI calibrators and entity identification sequences and (ii) entity identification sequences and labelled molecular targets.

Alternatively, entity identification sequences and UEI-calibrators may be assembled through their compatible overhangs before amplification and, after fusion of droplets of the first and second sets, the amplified fragment comprising UEI calibrator and UEI barcodes is ligated to labelled molecular targets through compatible overhangs of i) UEI calibrators and labelled molecular targets or (ii) entity identification sequences and labelled molecular targets.

Preferably, at least some of molecular targets are nucleic acids and at least some probes comprise

a capture moiety which is a single stranded DNA region which drives the specific recognition of a nucleic acid molecular target through conventional Watson-Crick base-pairing interactions and

a DNA moiety comprising a 3′ single stranded region comprising the unique molecular identification (UMI) sequence and a 5′double-stranded region comprising the overhang or overhang producing restriction site.

Preferably, said nucleic acid molecular targets are labelled using said probes as priming sites for a DNA polymerase synthesizing complementary strands of molecular targets.

In particular embodiments, at least some of molecular targets are RNA molecules and the DNA polymerase is a reverse transcriptase.

In some embodiments, at least some probes comprise a capture moiety which is

(i) a binding moiety that specifically binds to a molecular target and is directly bound to the DNA moiety,

(ii) a chimeric protein comprising a first domain that specifically binds to a molecular target and a second domain that binds to the DNA moiety, or

(iii) a binding moiety that binds specifically to a molecular target and a protein bridge, said protein bridge comprising a first domain that binds to the binding moiety and a second domain that binds to the DNA moiety.

Preferably, (i) the binding moiety or the first domain of the chimeric protein is selected from the group consisting of an antibody, a ligand of a ligand/anti-ligand couple, a peptide aptamer, a nucleic acid aptamer, a protein tag, or a chemical probe (e.g. suicide substrate) reacting specifically with a molecular target or a class of molecular targets, preferably is an antibody, (ii) the first domain of the protein bridge is an immunoglobulin-binding bacterial protein, preferably is domains A to E of protein A, and/or (iii) the second domain of the protein bridge or the chimeric protein is selected from the group consisting of SNAP-tag, CLIP-tag or Halo-Tag, preferably is a SNAP-tag.

In some preferred embodiments, at least some probes comprise a capture moiety comprising an antibody moiety specific to a molecular target and a protein bridge, said protein bridge comprising a first domain that binds to a Fc region of the antibody moiety and a second domain that binds to the DNA moiety, preferably a SNAP-tag.

The present invention also relates to a method of quantifying one or several molecular targets from a plurality of entities with single-entity resolution, said method comprising

labelling said molecular targets according to the method of the invention;

capturing said labelled molecular targets,

amplifying sequences comprising UMI and UEI barcodes, and optionally UEI-calibrator barcodes sequencing amplified sequences.

The sequencing of UMI and UEI barcodes, and optionally UEI-calibrator barcodes, allows to unambiguously assign each molecular target to a droplet/entity and thus to quantify the content of molecular targets in each droplet/entity.

Preferably, the entity is a cell, or a particle or an oil-in-water emulsion droplet exposing molecular targets on its outer surface. More preferably, the entity is a cell.

When the entity is a particle or an emulsion droplet exposing molecular targets on its outer surface, the method may further comprise
labelling molecular targets with probes as defined herein, and
encapsulating entities attached to labelled molecular targets within emulsion droplets, each droplet containing no more than one entity,

thereby obtaining the first set of emulsion droplets.

The present invention further relates to the use of a kit to label a plurality of molecular targets from a plurality of entities according to the method of the invention or to quantify one or several molecular targets from a plurality of entities with single-entity resolution according to the method of the invention, wherein the kit comprises

a microfluidic device comprising
a first emulsion re-injection module or on-chip droplet generation module;
a second emulsion re-injection module or on-chip droplet generation module
a droplet-pairing module, and
a module coupling droplet fusion to injection,

wherein emulsion re-injection modules and/or on-chip droplet generation modules are in fluid communication and upstream to the droplet-pairing module, the droplet-pairing module is in fluid communication and upstream to the module coupling droplet fusion to injection,

and optionally,
one or several probes as defined herein; and/or
one or several entity identification sequences as defined herein; and/or
one or several UEI-calibrators as defined herein; and/or
one or several primers suitable to amplify entity identification sequences and/or UEI-calibrators; and/or
an aqueous phase and/or an oil phase; and/or
a leaflet providing guidelines to use such a kit.

Alternatively, the invention relates to the use of a kit to label a plurality of molecular targets from a plurality of entities according to the method of the invention or to quantify one or several molecular targets from a plurality of entities with single-entity resolution according to the method of the invention, wherein the kit comprises
one or several probes as defined herein; and/or
one or several entity identification sequences as defined herein; and/or
one or several UEI-calibrators as defined herein; and/or
one or several primers suitable to amplify entity identification sequences and/or UEI-calibrators; and/or
an aqueous phase and/or an oil phase; and/or
a microfluidic device, preferably a microfluidic device of the invention, and optionally
a leaflet providing guidelines to use such a kit.

### BRIEF DESCRIPTION OF THE DRAWINGS

FIG. 1: Droplet-based microfluidics platforms for single cell molecular labeling. A. Single cell individualization and lysis. An aqueous stream containing the cells is combined with a stream of aqueous solution containing a lysis agent and optionally a double strand specific DNase. The emulsion is generated, collected and incubated to allow cell lysis and DNA degradation to occur. B. Droplet fusion. Droplets containing cell lysate are reinjected into a droplet fusion microfluidic chip and synchronized with on-chip generated droplets containing labeling mixture (reverse transcription mixture, DNA probes, antibodies . . . ). Pairs of droplets are then fused when passing between a pair of electrodes at the fusion point (arrow).

FIG. 2: Exemplary embodiment of synthetic DNA-based probe for targeted labeling of nucleic acids. In this example, the sequence of the probe designed to quantify RNA III of Staphylococcus aureus.

FIG. 3: Exemplary embodiment with a DNA probe comprising a capture moiety driving the specific recognition of a DNA adaptor linked to RNA molecular targets. The pre-adenylated adaptor (5′-App oligonucleotide) acts as a substrate for T4 ligase and is thus ligated to RNA molecules. The capture moiety of the probe then specifically hybridizes with the DNA adaptor.

FIG. 4: Exemplary embodiments with a DNA probe comprising a capture moiety which is a 5′ single stranded DNA region comprising 5′,5′-adenyl pyrophosphoryl moiety (App) onto its 5′-end. Such moiety acts as a substrate for T4 ligase and is thus ligated to RNA molecules.

FIG. 5: Exemplary embodiment of chimeric probe. A. Aptamer-based probe. This probe is composed of a RNA or DNA aptamer specific of the target molecule and fused to a synthetic DNA labeling moiety. B. Chimeric probe comprising a capture moiety comprising a protein bridge (SNAP-Tag and protein A) and an antibody specific of the molecular target. C. Schematic organization of the capture moiety.

FIG. 6: Exemplary embodiment of entity identification sequence. The entity identification sequence comprises a constant region, a unique restriction site, a UEI barcode and a sequencing primer annealing sequence.

FIG. 7: Multiple encapsulations of entity identification sequences (UEI): consequences and correction using UEI-calibrators. Two scenarios are exemplified using the simplified case of 2 UEI and 2 calibrators encapsulated into the same compartment. A. Multiple encapsulations of UEI without calibrators. Two cells are encapsulated into distinct droplets together with 2 different UEI. Upon UEI addition, target molecules from the same cell are associated with 2 different UEI. This will lead to the misassignment of target molecules to clusters representing only a sub-fraction of the cell and the integrity of the single-cell information will be compromised. B. Multiple encapsulations of UEI in the presence of calibrators. Two cells are encapsulated into distinct droplets together with 2 different UEI and 2 different calibrators. Upon UEI addition, both target molecules from the same cell and Calibrators are associated with 2 different UEI. At the end of the process, a first analysis defines the clusters of UEI associated with the same set of Calibrators. Target molecules labeled by UEI belonging to the same cluster are then clustered together and the integrity of the single-cell information is preserved.

FIG. 8: Exemplary embodiment with UEI-calibrators comprise two overhang producing restriction sites (RS), a first restriction site generating an overhang compatible with overhangs of digested entity identification sequences comprising UEI barcodes and a second restriction site generating an overhang compatible with overhangs of labelled molecular targets. In this embodiment, the digestion/ligation step used for UEI addition to labelled molecular targets, leads to the formation of tripartite molecules comprising a labelled molecular target, a UEI barcode and a UEI-calibrator barcode.

FIG. 9: Exemplary embodiment of UEI-Calibrator. The molecule is shown as a PCR-amplification product (double-stranded DNA). The UEI-Calibrator comprises a sequencing primer annealing sequence, a spacer, a UEI-calibrator barcode (UEI calibrator), a unique restriction site and a constant region comprising a primer binding site allowing amplification of UEI calibrator.

FIG. 10: Exemplary embodiment of a microfluidic device suitable to implement the method of the invention. The first set and second set of emulsion droplets are reinjected into the device (left micrographs), spaced with oil streams and synchronized at the junction of both reinjection channels. Synchronized droplets are allowed to circulate into a long channel having a width larger than the small droplet but narrower than the large droplet. As a consequence, the small droplet catches the large one and pairs of droplets (middle micrograph) are formed at the exit of the channel. Finally pairs of droplets are at the injection point where the enzyme solution is infused and the presence of electrodes trigger both droplets fusion and enzyme delivery (right micrograph).

FIG. 11: Co-flow droplet generator. The key dimensions of the microfluidic device are indicated. The depth of the channels was 10 μm.

FIG. 12: Droplet fluorescence analyzer. The key dimensions of the microfluidic device are indicated. The depth was 15 μm. Fluorescence measurement point is indicated by the open arrow.

FIG. 13: Fluorescence profile of orange-labelled droplets containing intact or lysed bacteria. Top panel: fluorescence profile of droplet containing intact bacteria. Each orange peak corresponds to a droplet. Green spikes observed into each orange peak corresponds to a fluorescent particle, therefore an intact bacterium. Bottom panel: fluorescence profile of droplet containing lyzed bacteria. Each orange peak corresponds to a droplet. Moreover, the presence of homogeneous green having the same width as the orange peak (e.g. the second peak from the left) indicates that the fluorescently-labelled nucleic acids have been released into the droplet, so that the bacterium has been lyzed.

FIG. 14. Bright-field and green fluorescence imaging of the water-in-oil droplets. The bacteria (green particles, arrows) encapsulated in the presence of CutSmart® buffer but in the absence of B-PER™ are shown on the left side whereas the bacteria encapsulated in the presence of B-PER™ are shown on the right side. Note that the lower number of fluorescent droplets after bacteria lysis is due to the more than a thousand-fold dilution of the fluorescence in the droplets following bacteria lysis, making these droplets difficult to distinguish from the background.

FIG. 15: Main steps in Unique Identifiers (UI) preparation.

FIG. 16: Analysis of PCR amplification and (co)-amplification of UEI-Calibrators and UEIs. Left panel: analysis of the PCR amplification of the UEIs (lane 1), the UEI-Calibrators (lane 2) or of both together (lane 3). Right panel: analysis of the PCR co-amplification of UEI-Calibrators and UEIs in bulk (lane 4) and in droplets (lane 5). The position of the expected size for UEI-Calibrators and of the UEIs amplification products size are labelled respectively by an open and a closed arrow. The lane L corresponds to the low range ladder (SM1203, Fermentas). Both gels were 8% native polyacrylamide-TBE 1× gels.

FIG. 17: Droplet generator. The key dimensions of the microfluidic device are indicated and the channels were 40 μm deep.

FIG. 18. Droplet picoinjector. Key dimensions are indicated and the channels were 40 μm deep. Ground and positive electrodes are shown in light and dark gray respectively.

FIG. 19. Analysis of DNA labelling efficiency. Top panel: The proper formation of an UI following the recombination of a UEI-Calibrator-bearing DNA (black square) with an UEI-bearing DNA (dashed squares) at the level of a restriction site (open square) brings annealing sites of primer 6 and 10 on the same DNA allowing for qPCR to take place. Middle panel: The Ct values are given for experiments performed both in bulk and in emulsion at both UEI-Calibrator/UEI ratios. Moreover, labelling reactions were performed in the presence (+) or in the absence (−) of restriction/ligation enzymes. Finally, for each reaction the number of the corresponding lane on analysis gel is given. Bottom left panel: analysis of qPCR products on 8% native polyacrylamide-TBE 1×. The lane L corresponds to the low range ladder (SM1203, Fermentas). The position of the product of expected size is indicated by the black arrow. Bottom right panel: gel purification of indexed library. The library of indexed UIs was purified on a 1% native agarose gel in TBE. The lane L corresponds to the 1 kb ladder (SM1163, Fermentas). The position of the product of expected size is indicated by the black arrow. The white dotted line box shows the band recovered for sequencing.

FIG. 20. Bioinformatics algorithm used to analyze sequencing data.

FIG. 21: Barcodes distribution and signature occurrence in droplets. Left panel: Distribution of UEI-Calibrators and UEIs in droplets. The distribution of the number of different barcode sequences per droplet is shown for the UEI-Calibrator (gray dashed bars) as well as for UEIs (open bars). Right panel: upon UIs clustering in Signatures, the occurrence of signature in the sequence pool was determined.

FIG. 22. Analysis of UI formation at various barcode lambda values. Left panel: qPCR analysis of UI formation. The proper formation of an UI upon the recombination of a UEI-Calibrator-bearing DNA (black square) with an UEI-bearing DNA (dashed squares) at the level of a restriction site (open square) brings annealing sites of primer 6 and 10 on the same DNA allowing for qPCR to take place. Ct values are given for the different lambda (number of different UEI-Calibrators and UEIs per droplet) tested. Right panel: analysis of qPCR products on 8% native polyacrylamide-TBE 1×. The lane L corresponds to the low range ladder (SM1203, Fermentas). The position of the product of expected size is indicated by the black arrow.

FIG. 23. Distribution profile of UEI-Calibrators and UEIs in the droplets. Occurrence at values 1 and 2 were intentionally removed as they contained significant sequencing noise.

FIG. 24: Scheme representation of the target DNA used.

FIG. 25: Analysis of UI formation and grafting to a target DNA. Left panel: qPCR analysis of UI formation and grafting to a target DNA. The proper formation of an UI upon the recombination of a UEI-Calibrator-bearing DNA (black square) with an UEI-bearing DNA (dashed squares) at the level of a restriction site (open square) as well as the attachment of the UI at an extremity of the target DNA (polka-dotted squares) mimicking a digested product brings annealing sites of primer 6 and 3 on the same DNA allowing for qPCR to take place. Ct values are given in the table. Right panel: analysis of qPCR products on 8% native polyacrylamide-TBE 1×. The lane L corresponds to the low range ladder (SM1203, Fermentas). The position of the product of expected size is indicated by the black arrow.

FIG. 26. Schematic workflow of UI preparation and target DNA labelling in droplets.

FIG. 27. Barcoding chip. Key dimensions are indicated and the channels were 40 μm deep. Ground and positive electrodes are shown in light and dark gray respectively.

FIG. 28. Purification gel of the Target DNA-UI indexed library. The indexed library was purified on a 1% native agarose gel in TBE. The lane L corresponds to the 1 kb ladder (SM1163, Fermentas). The position of the product of expected size is indicated by the black arrow. The white dotted line box shows the band recovered for sequencing.

FIG. 29. Bioinformatics algorithm used to deconvolute sequencing data and determine the copy number of target molecules per droplet/cell.

FIG. 30. Droplet-to-droplet reliability. The number of DNA molecules identified per droplet is represented for each droplet. Note that droplets with low DNA content (between 1.8 and 0.5) were likely to be noise but were kept in the analysis to not bias it.

FIG. 31. Scheme representation of the primer used to reverse transcribed the RNA-III.

FIG. 32. 2 μL droplet generator. The key dimensions of the devices are indicated. The channels were 10 μm deep.

FIG. 33. Microfluidic droplet fuser. Key dimensions are indicated and the channels were 15 μm deep. Ground and positive electrodes are shown in light and dark gray respectively.

FIG. 34. Analysis of reverse transcription products. Top: upon reverse transcription, the RT primer is extended and contain annealing site of primer 21. The generated cDNA contains both primer-binding sites (20 and 21) and can be detected by qPCR. Ct values are given in the table. Bottom: analysis of qPCR products on 8% native polyacrylamide-TBE 1×. Gels on the left, the middle and the right correspond respectively to the experiment started with 1000, 100 and 10 RNA per droplet. Lanes 1, 4 and 7 are the negative controls, lanes 2, 5 and 8 correspond to the experiment performed in bulk and lanes 3, 6 and 9 correspond to the reaction performed in droplets. The lanes L correspond to the low range ladder (SM1203, Fermentas). The position of the product of expected size is indicated by the black arrows whereas small parasitic side products are shown by the open arrows.

FIG. 35. Analysis of PCR products on 1% agarose gel-TBE. UI were initially prepared with random region-free (lane 1), N2×5 (lane 2), N4443 (lane 3) and N15 (lane 4) templates. The position of the product of expected size is indicated by the black arrow. The black vertical bar shows the short parasitic side products.

FIG. 36. Workflow of the preparation of NaBAb-DNA/IgG complex.

FIG. 37. Preparation of NaBAb-DNA/IgG complex. Left panel: Incubation of BG-labelled fluorescent DNA with (lane 2) and without (lane 1) NaBAb protein. L indicates a molecular weight ladder. Right panel: NaBAb protein was incubated alone (lane 1), with BG-labelled fluorescent DNA (lane 2) as well as an increasing concentration of IgG (62.5 μg/mL on lane 3, 112 μg/mL on lane 4 and 225 μg/mL on lane 5). Upon incubation, the reaction products were loaded on a native polyacrylamide gel and the position of DNA molecule was revealed by imaging gel fluorescence (emitted by the Alexa488 conjugated with the DNA) without further staining.

FIG. 38. UI attachment to NaBAb-DNA complex. Left panel: qPCR analysis of UI grafting to DNA-labelled protein. The proper formation of an UI upon the recombination of a UEI-Calibrator-bearing DNA (black square) with an UEI-bearing DNA (dashed squares) at the level of a restriction site (open square) as well as the attachment of the UI, via a compatible restriction site at the extremity of a DNA covalently attached to a protein brings annealing sites of primer 6 and 10 on the same DNA allowing for qPCR to take place. Ct values are given in the table. Right panel: analysis of qPCR products on 1% agarose gel-TBE 1×. The lane L corresponds to the low range ladder (SM1203, Fermentas). The position of the product of expected size is indicated by the black arrow.

### DETAILED DESCRIPTION OF THE INVENTION

The inventors conceived a new method of labelling any target molecules from a plurality of entities in high throughput regimes, i.e. allowing the analysis of several thousands of entities per run, while preserving the integrity of the single-entity information. This method is based on a tandem molecular barcoding in which all molecular targets (nucleic acids, proteins, . . . ) are labelled (i) with a first unique barcode (unique molecular identification barcode or UMI barcode) which is different for each molecular target from an entity, and (ii) with a tag sequence coding the entity from which the molecular target originates, i.e. unique entity identification barcode or UEI barcode which is different for each entity but identical for all molecular targets originating from the same entity. Once this tandem barcoding is performed, the absolute quantification of all molecular targets with a single-entity resolution may be carried out in a single run of next-generation sequencing making this method highly sensitive and cost-effective.

In a first aspect, the present invention relates to a method of labelling a plurality of molecular targets from a plurality of entities, said method comprising providing a first set of emulsion droplets comprising droplets containing labelled molecular targets, wherein each of these droplets contains a plurality of molecular targets originating from no more than one entity and wherein, in each of these droplets, each molecular target is labelled with a molecular identification DNA sequence comprising (i) a unique molecular identification (UMI) barcode which is different for each molecular target and (ii) an overhang or an overhang producing restriction site;

providing a second set of emulsion droplets comprising droplets containing entity identification sequences, wherein each of these droplets contains at least one entity identification sequence which is a DNA sequence, preferably a double-stranded DNA sequence, comprising a unique entity identification (UEI) barcode which is different for each droplet of the second set, and an overhang producing restriction site;

fusing droplets of the first set with droplets of the second set wherein a droplet of the first set is fused with no more than one droplet of the second set; and

ligating UEI barcodes to labelled molecular targets, optionally after restriction enzyme digestion.

The method of the invention may be used to label molecular targets from any type of entities.

As used herein, the term “entity” refers to any entity comprising or exposing on its surface, molecular targets as defined below. In particular, this term refers to a cell, or refers to a particle or an emulsion droplet, preferably an oil-in-water emulsion droplet, exposing molecular targets on its outer surface.

As used herein, the term “cell” refers to a prokaryotic cell or a eukaryotic cell such as animal, plant, fungal or algae cell. The population of cells to be processed may be homogenous, i.e. comprising only one cellular type, or may be heterogeneous, i.e. comprising several cellular types. In some embodiments, the population of cells is obtained from a tissue sample, preferably an animal tissue sample, more preferably from a pathological sample such as a tumor sample. In some other embodiments, the population of cells is a population of bacterial, fungal or algae cells, preferably of bacterial or fungal cells. This population may comprise bacteria, fungi or algae of the same species or bacteria, fungi or algae of different species.

The terms “particle” and “bead” are used herein interchangeably and refer to any solid support, preferably a spherical solid support, of 50 nm to 10 μm in size which is suitable to expose one or several molecular targets on its outer surface. In particular, these terms may refer to polymer beads (e.g. polyacrylamide, agarose, polystyrene), latex beads, magnetic beads or hydrogel beads. Methods for covalent or non-covalent binding of molecular targets such as nucleic acids or proteins, to beads are well known by the skilled person and various techniques are commercially available. In particular, this binding may be carried out through reactive groups on the surface of the particle. For example, nucleic acids may be attached to the surface by carbodiimide-mediated end-attachment of 5′-phosphate and 5′-NH2 modified nucleic acids to respectively amino and carboxyl beads. Proteins may also be covalently or non-covalently attached to beads via any suitable method such as using sulphate, amidine, carboxyl, carboxyl/sulphate or chloromethyl modified beads.

The term “entity” may also refer to an emulsion droplet, preferably an oil-in-water emulsion droplet, exposing molecular targets on its outer surface. Molecular targets may be covalently or non-covalently attached to the droplet through reactive groups exposed on the surface of the droplets such as nitrilotriacetate which can specifically interact with his-tagged proteins, or through any other functional moiety which is able to covalently or non-covalently interact with a molecular target of interest. The skilled person may use any known method to produce such emulsion droplets exposing molecular targets on its outer surface, in particular methods described in international patent application WO 2017/174610.

The method of the invention allows labelling molecular targets from a high number of entities in a single run. Thus, as used herein, the term “plurality of entities” refers to at least 1,000 entities, preferably at least 5,000 entities, more preferably at least 10,000 entities, and even more preferably at least 50,000 entities.

As used herein, the term “target molecule” or “molecular target” refers to any kind of molecules, and in particular any kind of molecules which may be possibly present in a cell. The molecular target can be a biomolecule, i.e. a molecule that is naturally present in living organisms, or a chemical compound that is not naturally found in living organism such as pharmaceutical drugs, toxicants, heavy metals, pollutants, etc. . . . . Preferably, the molecular target is a biomolecule. Examples of biomolecules include, but are not limited to, nucleic acids, e.g. DNA or RNA molecules, proteins such as antibodies, enzymes or growth factors, lipids such as fatty acids, glycolipids, sterols or glycerolipids, vitamins, hormones, neurotransmitters, and carbohydrates, e.g., mono-, oligo- and polysaccharides. The terms “polypeptide”, “peptide” and “protein” are used interchangeably to refer to a polymer of amino acid residues, and are not limited to a minimum length. The protein may comprise any post-translational modification such as phosphorylation, acetylation, amidation, methylation, glycosylation or lipidation. As used herein, the term “nucleic acid” or “polynucleotide” refers to a polymeric form of nucleotides of any length, either ribonucleotides or deoxyribonucleotides.

One of the main advantage of the method of the invention, is the possibility to label, in a single run, different types of molecular targets such as proteins and nucleic acids. Thus, the term “plurality of molecular targets” may refer to different copies of the same molecule, e.g. different copies of the same mRNA or of the same protein, or may refer to different copies of different molecules, e.g. different copies of a mRNA and different copies of a protein.

In an embodiment, molecular targets are different copies of the same molecule. Preferably the molecule is biomolecule, more preferably a nucleic acid or a protein, even more preferably a RNA molecule or a protein. In another embodiment, molecular targets are different copies of different molecules. Preferably said molecules are biomolecules, more preferably are nucleic acids and/or proteins, even more preferably RNA molecules and/or proteins. In a particular embodiment, molecular targets are different copies of at least two different nucleic acids, preferably RNA. In another particular embodiment, molecular targets are different copies of at least two different proteins. In a further embodiment, molecular targets are different copies of one or several nucleic acid, preferably RNA, and different copies of one or several proteins.

In preferred embodiments, the method of the invention is implemented using one or several microfluidic systems, i.e. at least one step of the method is implemented using a microfluidic system. In some embodiments, the method is implemented using several microfluidic systems, for example a microfluidic system to generate the first set of emulsion droplets, a microfluidic system to generate the second set of emulsion droplets and a microfluidic system to fuse the two sets, to incorporate UEI barcodes and optionally to conduct some subsequent steps. In some other embodiments, the method is implemented using a microfluidic system wherein the first set of emulsion droplets and/or the second set of emulsion droplets are generated and wherein droplets of the two sets are fused.

As used herein, the terms “emulsion droplet”, “droplet” and “microfluidic droplet” are used interchangeably and may refer to a water-in-oil emulsion droplet (also named w/o droplet), i.e. an isolated portion of an aqueous phase that is completely surrounded by an oil phase, an oil-in-water emulsion droplet (also named o/w droplet), i.e. an isolated portion of an oil phase that is completely surrounded by an aqueous phase, a water-in-oil-in-water emulsion droplet (also named w/o/w droplet) consisting of an aqueous droplet inside an oil droplet, i.e. an aqueous core and an oil shell, surrounded by an aqueous carrier fluid, or an oil-in-water-in-oil emulsion droplet (also named o/w/o droplet) consisting of an oil droplet inside an aqueous droplet, i.e. an oil core and an aqueous shell, surrounded by an oil carrier fluid. Preferably, this term refers to a w/o emulsion droplet.

A droplet may be spherical or of other shapes depending on the external environment. Typically, the droplet has a volume of less than 100 nL, preferably of less than 10 nL, and more preferably of less than 1 nL. For instance, a droplet may have a volume ranging from 2 pL to 1 nL, preferably from 2 to 500 pL, more preferably from 2 to 100 pL. Preferably, the droplets have a homogenous distribution of diameters, i.e., the droplets may have a distribution of diameters such that no more than about 10%, about 5%, about 3%, about 1%, about 0.03%, or about 0.01% of the droplets have an average diameter greater than about 10%, about 5%, about 3%, about 1%, about 0.03%, or about 0.01% of the average diameter of the droplets. Preferably, the emulsion is a monodispersed emulsion, i.e. an emulsion comprising droplets of the same volume. Techniques for producing such a homogenous distribution of diameters are well-known by the skilled person (see for example WO 2004/091763).

The aqueous phase is typically water or an aqueous buffer solution, such as but not limited to Tris-HCl buffer, Tris-acetate buffer, phosphate buffer saline (PBS) or acetate buffer. Preferably, the aqueous phase is an aqueous buffer solution. Optionally, the aqueous phase may comprise bovine serum albumin or additive such as Pluronic. In preferred embodiments, the aqueous phase is chosen in order to be compatible with enzymatic reactions performed during the process of the invention, such as enzymatic digestion, amplification, ligation, etc. . . . . An example of such aqueous phase includes, but is not limited to, CutSmart restriction enzyme buffer (New England Biolabs).

The oil phase used to generate the emulsion droplets may be selected from the group consisting of fluorinated oil such as FC40 oil (3M®), FC43 (3M®), FC77 oil (3M®), FC72 (3M®), FC84 (3M®), FC70 (3M®), Novec-7500 (3M®), Novec-7100 (3M®), perfluorohexane, perfluorooctane, perfluorodecane, Galden-HT135 oil (Solvay Solexis), Galden-HT170 oil (Solvay Solexis), Galden-HT110 oil (Solvay Solexis), Galden-HT90 oil (Solvay Solexis), Galden-HT70 oil (Solvay Solexis), Galden PFPE liquids, Galden® SV Fluids or H-Galden® ZV Fluids; and hydrocarbon oils such as Mineral oils, Light mineral oil, Adepsine oil, Albolene, Cable oil, Baby Oil, Drakeol, Electrical Insulating Oil, Heat-treating oil, Hydraulic oil, Lignite oil, Liquid paraffin, Mineral Seal Oil, Paraffin oil, Petroleum, Technical oil, White oil, Silicone oils or Vegetable oils. Preferably, the oil phase is fluorinated oil such as Novec-7500, FC40 oil, Galden-HT135 oil or FC77 oil, more preferably is Novec-7500. The skilled person may easily select suitable phase oil to implement the methods of the invention.

The emulsion droplets comprise one or several surfactants. Said surfactant(s) can aid in controlling or optimizing droplet size, flow and uniformity and stabilizing aqueous emulsions. Suitable surfactants for preparing the emulsion droplets used in the present invention are typically non-ionic and contain at least one hydrophilic head and one or several lipophilic tails, preferably one (diblock surfactant) or two (triblock surfactant) lipophilic tails. Said hydrophilic head(s) and the tail(s) may be directly linked, or linked via a spacer moiety. Examples of suitable surfactants include, but are not limited to, sorbitan-based carboxylic acid esters such as sorbitan monolaurate (Span 20), sorbitan monopalmitate (Span 40), sorbitan monostearate (Span 60) and sorbitan monooleate (Span 80); block copolymers of polyethylene glycol and polypropylene glycol such as the triblock copolymer EA-surfactant (RainDance Technologies), DMP (dimorpholino phosphate)-surfactant (Baret, Kleinschmidt, et al., 2009) and Jeffamine-surfactant; polymeric silicon-based surfactants such as Abil EM 90; triton X-100; and fluorinated surfactants such as PFPE-PEG and perfluorinated polyethers (e.g., Krytox-PEG, DuPont Krytox 157 FSL, FSM, and/or ESH). In the context of the invention, preferred surfactants are fluorinated surfactants, i.e. fluorosurfactants.

In some particular embodiments, the emulsion droplets comprise one or several functionalized surfactants at their interface. As used herein, a “functionalized surfactant” refers to a surfactant which bears at least one functional moiety either on one of its hydrophilic head(s) or lipophilic tail(s), preferably on a hydrophilic head. As used herein, a “functional moiety” is virtually any chemical or biological entity which provides the surfactant with a function of interest. For instance, the functional moiety can enable to create a covalent or non covalent interaction between the surfactant and a molecular target of interest. Thanks to the use of such functionalized surfactants, molecular targets may be exposed on the surface of emulsion droplets. The interface of these droplets may comprise only functionalized surfactant(s) or a mix of functionalized and non-functionalized surfactants. The ratio between functionalized and non-functionalized surfactants may vary and can be easily adapted by the skilled person. For example, functionalized surfactant may represent from 1 to 100% (w/w) of total surfactants, preferably from 2 to 80% (w/w), and more preferably from 5 to 50% (w/w).

The total amount of surfactant in the carrier oil is preferably chosen in order to ensure stability of the emulsion and prevent spontaneous coalescence of droplets. Typically, the carrier oil comprises from 0.5 to 10% (w/w), preferably from 1 to 8% (w/w), and more preferably from 2 to 5% (w/w) of surfactant.

The emulsion can be prepared by any method known by the skilled artisan. Preferably, the emulsion can be prepared on a microfluidic system.

Providing the First Set of Emulsion Droplets Comprising Labelled Molecular Targets

The first set of emulsion droplets comprises droplets containing labelled molecular targets, wherein each of these droplets contains a plurality of molecular targets originating from no more than one entity and wherein, in each of these droplets, molecular targets are labelled with a molecular identification DNA sequence.

The first set of emulsion droplets may be a w/o or o/w/o emulsion depending on the nature of the entities. In some embodiments, the entities are cells or particles and emulsion droplets of the first set are w/o emulsion droplets. In some other embodiments, the entities are o/w emulsion droplets exposing molecular targets on their outer surfaces and emulsion droplets of the first set are o/w/o emulsion droplets.

Preferably, the first set of emulsion droplets comprises at least 10,000 droplets, preferably at least 100,000 droplets, preferably at least 500,000 droplets, and even more preferably at least 1,000,000 droplets.

In some embodiments, the first set of emulsion droplets is obtained by
encapsulating entities within emulsion droplets, each droplet containing no more than one entity, and optionally lysing said entities within the droplets to release molecular targets and
labelling said molecular targets.

In particular embodiments, entities are particles or o/w emulsion droplets exposing molecular targets on their outer surfaces, and the first set of emulsion droplets is obtained by
encapsulating entities within emulsion droplets, each droplet containing no more than one entity, and
labelling said molecular targets.

In some particular embodiments wherein entities are particles or o/w emulsion droplets exposing molecular targets on their outer surfaces, labelling of molecular targets may be performed in bulk, and then entities attached to labelled molecular targets may be encapsulated.

Thus, when the entity is a particle or an emulsion droplet exposing molecular targets on its outer surface, the method may comprise
labelling said molecular targets, and
encapsulating entities attached to labelled molecular targets within emulsion droplets, each droplet containing no more than one entity, thereby obtaining the first set of emulsion droplets.

In preferred embodiments, entities are cells and the first set of emulsion droplets is obtained by
encapsulating entities within emulsion droplets, each droplet containing no more than one entity,
lysing said entities within the droplets to release molecular targets, and
labelling said molecular targets.

To ensure single-entity resolution, entities have to be confined and isolated from the beginning to the end of the barcoding process. Encapsulation of entities into microfluidic droplets is a convenient way to isolate said entities and is particularly suitable to high throughput regimes.

Those of ordinary skill in the art is aware of techniques for encapsulating cells or particles within microfluidic droplets (see, for example, the international patent application WO 2004/091763 incorporated herein by reference). If cells are adherent or from tissue, they may be first dissociated and optionally filtered or centrifuged to remove clumps of two or more cells before encapsulation. Cells may typically be suspended in an aqueous buffer such as PBS buffer.

Methods for producing monodisperse w/o/w double emulsions, i.e. for encapsulating o/w droplets are also well known by the skilled person and microfluidic systems generating such emulsions are commercially available.

During encapsulation, the entity number density (entities per unit volume) has to be adjusted to minimize incidences of two or more entities becoming captured in the same droplet. In particular, entities may be encapsulated at a density of less than 1 entity per droplet, preferably at a density of less than 0.2 entity per droplet, in order to prevent co-encapsulation of two or more entities. In preferred embodiments, the entity number density and the average occupancy is adjusted in order to ensure that most, preferably at least 98%, or all of the droplets have only zero or one entity present in them.

Using any well-known microfluidic method to encapsulate entities into microfluidic droplets, entity-bearing droplets may be produced at high frequency, e.g. ranging from 0.5 kHz to 15 kHz, preferably from 1 kHz to 10 kHz, more preferably from 1 kHz to 5 kHz.

In embodiments wherein entities are cells, after encapsulation, cells may be lysed within the droplets in order to release molecular targets. Cell lysis may be performed using any method known by the skilled person such as using physical, chemical or biological means. In particular, cells may be lysed using radiation (e.g. UV, X or y-rays) or laser (see e.g. Rau et al., 2004). The lysis may also be induced by osmotic shock or by addition of a detergent or enzyme (see, e.g. Kintses et al., 2012; Novak et al., 2011; Brown & Audet, 2008). The lysis may also be induced by heat shock.

In some embodiments, the lysis is induced by a lysis agent. Preferably, the lysis agent comprises one or several components altering the osmotic balance, one or several detergents and/or one or several enzymes. More preferably, the lysis agent is Triton X-100, BugBuster® reagent (Merck Millipore), Nonidet P40™ (MP BioMedical), M-PER™ (Thermo Scientific) or B-PER™ (Thermo Scientific).

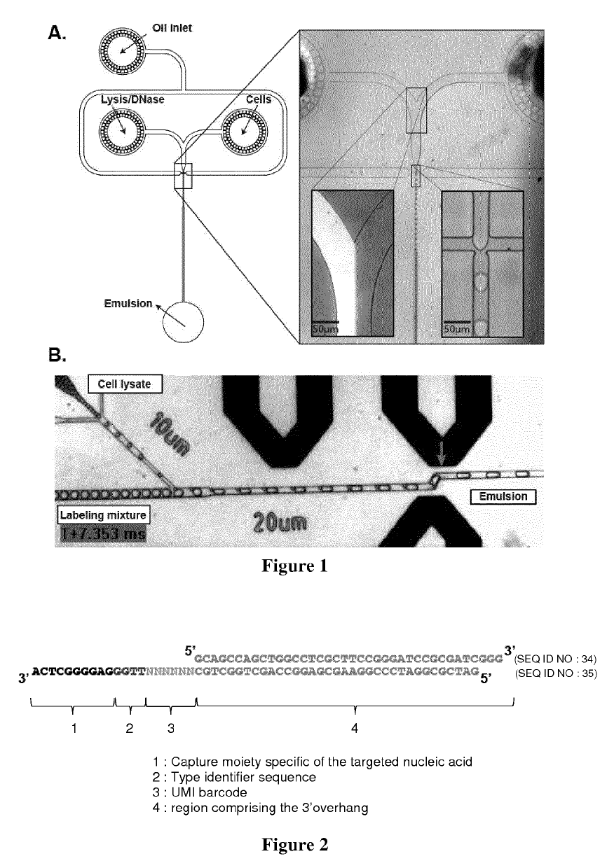

In an embodiment, the lysis agent is directly added to the aqueous phase of the droplets before encapsulation. In such embodiment, an aqueous stream containing the cells may be combined with a stream of aqueous solution containing the lysis agent just before generation of droplets (see, e.g. FIG. 1A). The emulsion may be then generated, collected and incubated to allow cell lysis.

In another embodiment, the lysis agent is introduced inside the droplet after droplet generation by any known technique such as pico-injection or droplet fusion. The emulsion may be then collected and incubated to allow cell lysis.

Typically, the emulsion is incubated from 5 minutes to 1 hour and at a temperature ranging from 4° C. to 25° C. to allow cell lysis.

Alternatively, the lysis may be induced by a heat treatment. Typically, in this case, the emulsion may be incubated from 5 minutes to 1 hour and at a temperature up to 95° C. to allow cell lysis.

The skilled person can easily adapted the incubation temperature during the lysis to the used method.

In some embodiments wherein the w/o interface of the first set droplets comprises functionalized surfactant(s), some or all molecular targets released by cell lysis, may be bound by said surfactant(s) and concentrated onto the inner w/o interface of droplets. Possibly, these w/o droplets can be convert into o/w droplets using droplet inversion as presented in international patent application WO 2017/174610.

In some embodiments wherein entities are particles or o/w emulsion droplets exposing molecular targets on their outer surfaces, molecular targets can be released from said particles or from the surface of said o/w emulsion droplets by the action of a cleaving agent (e.g. restriction enzyme).

Optionally, depending on the nature of molecular targets, one or several additional reagents may be added to the aqueous phase before collection and incubation of the first set emulsion. Examples of such additional reagents may include, but are not limited to DNases, RNases, proteases, protease inhibitors and/or nuclease inhibitors.

In some embodiments, molecular targets are RNA and one or several additional reagents, preferably comprising one or several DNases and/or one or several proteases, are added to the aqueous phase. In some other embodiments, molecular targets are proteins and/or RNA and additional reagents, preferably comprising one or several DNases, are added to the aqueous phase.

Additional reagent(s) and lysis agent may be added simultaneously to the aqueous phase, i.e. directly added to the aqueous phase of the droplets just before encapsulation or after droplet generation by any known technique such as pico-injection or droplet fusion. Alternatively, additional reagent(s) and lysis agent may be added sequentially. In particular, the lysis agent may be added to the aqueous phase before encapsulation by co-flowing a flow of an aqueous solution containing the entities and a flow of a solution containing the lysis agent, and additional reagent(s) may be added after encapsulation, and vice-versa, or the lysis agent and additional reagent(s) may be added sequentially after encapsulation, e.g. separate pico-injection or droplet fusion.

In preferred embodiments, additional reagent(s) and lysis agent are added simultaneously to the aqueous phase, i.e. directly added to the aqueous phase of the droplets just before encapsulation (see, e.g. FIG. 1A).

After encapsulation, and optionally cell lysis and/or elimination/degradation of some non-targeted molecules, as detailed above, molecular targets are labelled with a molecular identification DNA sequence comprising (i) a unique molecular identification (UMI) barcode which is different for each molecular target and (ii) an overhang or an overhang producing restriction site.

The “UMI sequence” or “UMI barcode” is a randomized nucleotide sequence assigning a unique barcode to each molecular target and thus allows further performing the digital detection/counting of molecular targets initially present into or onto the entity, their absolute quantification and correcting for amplification biases. Indeed, in a droplet, each probe carries a unique identification number (the UMI) and therefore counting the number of different UMI gives the absolute number of labelled molecular targets. Preferably, the UMI sequence is a randomized nucleotide sequence having a length of at least 5 nucleotides, preferably a length from 5 to 15 nucleotides, more preferably a length from 5 to 10 nucleotides. The randomized sequence can be a stretch of contiguous randomized nucleotides or a stretch of semi-randomized nucleotides (i.e. contiguous randomized nucleotides spaced by constant nucleotides). Typical examples of a stretch of semi-randomized nucleotides are stretches where several randomized dinucleotides are spaced by constant dinucleotides, or stretches where several randomized trinucleotides are spaced by constant trinucleotides. Preferably, the UMI sequence is a stretch of semi-randomized nucleotides, in particular a stretch where several randomized dinucleotides are spaced by constant dinucleotides.

An overhang is required to add the UEI barcode and optionally the UEI-calibrator barcode, as detailed below, after fusion with the droplets of the second set. This overhang may be a 3′overhang or a 5′overhang, preferably is a 3′overhang.

The molecular identification DNA sequence may comprise an overhang (3′ or 5′ overhang) compatible with a cohesive end generated by a restriction enzyme, or may comprise an overhang producing restriction site.

In some embodiments, the molecular identification DNA sequence comprises an overhang, preferably a 3′ overhang, compatible with a cohesive end generated by a restriction enzyme. The choice of this configuration ensures that no complementary strand will be synthesized by the filling activity of a polymerase so that the extremity will stay competent for the later UEI addition. In addition, this overhang is compatible with the cleavage product of the restriction enzyme which is used to digest double stranded DNA sequences bearing UEI barcodes or UEI calibrators and comprised in the droplets of the second set as detailed below. Using digestion and ligation, this overhang allows UEI, and optionally UEI-calibrator, addition to each UMI sequence.

In some other embodiments, the molecular identification DNA sequence comprises an overhang producing restriction site, i.e. a restriction site generating a 3′ or 5′overhang, preferably 3′overhang, which is compatible with the cleavage product of the restriction enzyme which is used to digest double stranded DNA sequences bearing UEI barcodes or UEI calibrators and comprised in the droplets of the second set. In a particular embodiment, the overhang producing restriction site on the molecular identification DNA sequence is recognized by the same enzyme than the overhang producing restriction site on double stranded DNA sequences bearing UEI calibrators (see below). In particular, overhangs on labelled molecular targets may be generated while digesting DNA sequences bearing UEI barcodes and DNA sequences bearing UEI calibrators after fusion of droplets of the first and second sets.

Alternatively, in embodiments wherein entities particles or o/w emulsion droplets exposing molecular targets on their outer surfaces, molecular targets may be released from said particles or from the surface of said o/w emulsion droplets by the action of a restriction enzyme, thereby generating a 3′ or 5′overhang. Optionally, the molecular identification DNA sequence may further comprise a type identifier sequence which is a short predefined sequence, preferably having a length from 4 to 8 nucleotides, coding for the nature (e.g. nucleic acid or protein) and/or the identity (e.g. GFP mRNA) of the molecular target. Preferably, the type identifier sequence is proximal to the UMI sequence, more preferably directly adjacent to the UMI sequence.

In preferred embodiments, labelling of molecular targets with UMI barcodes is performed using probes, each probe comprising

a capture moiety capable of specific binding to a molecular target or specific ligation to a molecular target or an adaptor linked to said molecular target and

a DNA moiety comprising the molecular identification DNA sequence, i.e. (i) a region proximal to the capture moiety and comprising the unique molecular identification (UMI) sequence, and optionally a type identifier sequence, and (ii) a region distal from the capture moiety and comprising an overhang, preferably an overhang compatible with a cohesive end generated by a restriction enzyme, or an overhang producing restriction site. Preferably the region distal from the capture moiety comprises a 3′overhang or a 3′overhang producing restriction site.

In embodiments wherein the region distal from the capture moiety comprises a 3′overhang, the 5′end is a phosphorylated 5′end.

Preferably, the restriction site or overhang is separated from the UMI sequence by at least 10 nucleotides, preferably at least 20 nucleotides, and more preferably 20 to 40 nucleotides. Preferably, this separating region has a melting temperature of at least 50° C., more preferably at least 55° C., is GC rich in order to form stable duplexes, does not exhibit any sequence identity with a nucleic acid found in the organism from which the cell is originated and does not contain one of the restriction sites later used for labelling. In preferred embodiments, this region is identical for all probes.

In a particular embodiment, the DNA moiety comprises, from the capture moiety to the restriction site or overhang, a type identifier sequence of 4 to 8 nucleotides, preferably of 4 nucleotides, a UMI sequence of 5 to 15 nucleotides, preferably of 8 nucleotides, and a region of 20 to 40 nucleotides comprising the restriction site or overhang.

The nature of the capture moiety and the structure of the DNA moiety may differ according to type and/or the chemical structure of molecular targets. Since molecular targets of different types and/or chemical structures may be labelled simultaneously, probes contained in the same droplet may have different structures.

In some embodiments, at least some of molecular targets are nucleic acids and at least some probes specific of said nucleic acids are DNA probes comprising

a capture moiety which is a single stranded DNA region which drives the specific recognition of a nucleic acid molecular target, or the specific recognition of a nucleic acid adaptor linked to said molecular target, through conventional Watson-Crick base-pairing interactions, and

a DNA moiety comprising (i) a 3′ single stranded region proximal to the capture moiety and comprising the unique molecular identification (UMI) sequence, and optionally a type identifier sequence, and (ii) a 5′double-stranded region distal from the capture moiety and comprising an overhang, preferably an overhang compatible with a cohesive end generated by a restriction enzyme, or an overhang producing restriction site.

Preferably, the DNA moiety comprises (i) a 3′ single stranded region proximal to the capture moiety and comprising the unique molecular identification (UMI) sequence, and optionally a type identifier sequence, and (ii) a 5′ double-stranded region distal from the capture moiety and comprising a 3′overhang, preferably compatible with a cohesive end generated by a restriction enzyme.

These DNA probes may be produced by any method known by the skilled person such as chemical synthesis.

An exemplary illustration of such a probe is presented in FIG. 2.

In these embodiments, the length of the capture moiety has to be sufficient to allow the specific recognition of the target molecule through hybridization. Preferably, the capture moiety is a single stranded DNA region of at least 8 nucleotide long, preferably of 8 to 25 nucleotide long, more preferably of 10 to 15 nucleotide long.

The melting temperature (Tm) of the perfect hybrid formed upon association of the capture moiety with the molecular target is preferably adjusted (e.g. by modulating the length of the sequence specific to the target nucleic acid) in order to be ranged between 30° C. and 70° C., preferably between 30° C. and 60° C., more preferably between 40° C. and 60° C., and even more preferably between 40° C. and 50° C.

Preferably, the difference between melting temperature (Tm) of all probes specific to nucleic acid molecular targets, is lower than 3° C., more preferably lower than 2° C. and even more preferably lower than 1° C.

The capture moiety may be specific to a particular DNA or RNA or may be complementary to a sequence region common to all RNAs, e.g. the capture moiety may be a poly-T tract which is complementary to the poly-A tails of eukaryotic mRNAs or complementary to a nucleic acid adaptor, preferably a DNA adaptor, added to all RNA, for instance through the action of an RNA ligase.

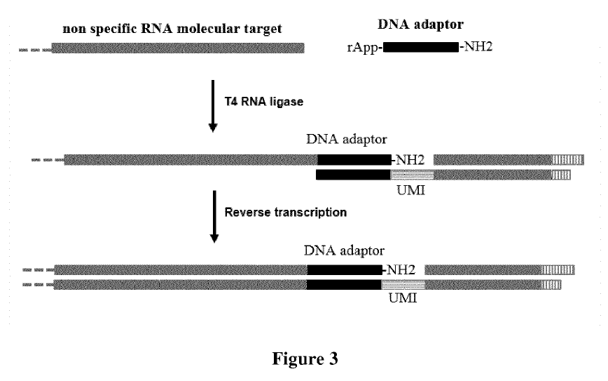

Thus, in some embodiments, the capture moiety drives the specific recognition of a nucleic acid adaptor linked to the molecular targets of interest, e.g. all RNAs. Such adaptor may be, for example, pre-adenylated oligonucleotides (5′-App oligos) which act as substrates for T4 ligases and thus can be ligated to any RNA molecule. Typically such adaptor may be a single stranded DNA region of at least 8 nucleotide long, preferably of 8 to 25 nucleotide, more preferably of 10 to 15 nucleotide long. An exemplary illustration of such embodiment is presented in FIG. 3.

In some other embodiments, at least some of molecular targets are nucleic acids and at least some probes specific of said nucleic acids are DNA probes comprising

a capture moiety which is a single stranded DNA region which is able to ligate to a nucleic acid molecular target, and

a DNA moiety comprising (i) a 5′ single stranded region proximal to the capture moiety and comprising the unique molecular identification (UMI) sequence, and optionally a type identifier sequence, and (ii) a 3′double-stranded region distal from the capture moiety and comprising an overhang, preferably an overhang compatible with a cohesive end generated by a restriction enzyme, or an overhang producing restriction site.

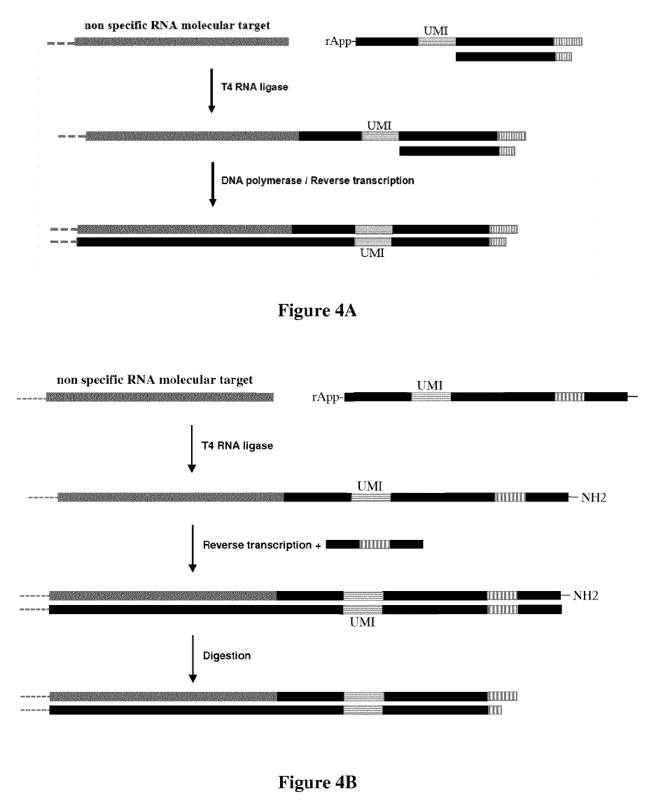

Preferably, the capture moiety is a 5′ single stranded DNA region comprising 5′,5′-adenyl pyrophosphoryl moiety (App) onto its 5′-end. Such moiety may act as substrate for T4 ligases and thus can be ligated to the 3′ end any RNA molecule. In this embodiment, the 5′App extremity of the capture moiety directly interacts with the RNA molecular target and ligates to said target in the presence of T4 ligase. Exemplary illustrations of such embodiment are presented in FIGS. 4A and 4B.

These DNA probes may be produced by any method known by the skilled person such as chemical synthesis.

Preferably, the capture moiety is specific to a particular DNA or RNA molecule, more preferably is specific to a particular RNA molecule.

In some embodiments, nucleic acid molecular targets may be labelled using probes as described above, and in particular the capture moiety of said probes, as priming sites for a DNA polymerase synthesizing complementary strands of molecular targets. DNA and/or RNA molecular targets are converted into barcoded complementary DNA (cDNA) upon reverse transcription or other DNA polymerization reaction.

In some embodiments, some molecular targets are RNA molecules and the DNA polymerase is a reverse transcriptase. Upon hybridization of the probe, through its capture moiety, to the targeted RNA or the adaptor, reverse transcription can occur and first strand of complementary DNA (cDNA) can be synthesized, said cDNA comprising the UMI sequence of the DNA moiety of the probe.

It will be recognized that DNA polymerization reaction or reverse transcription requires appropriate conditions, for example the presence of an appropriate buffer and DNA polymerase enzyme, temperatures appropriate for annealing of the probes to targeted RNAs or DNAs and the activity of the enzyme and optionally presence of DTT. These conditions mainly depend on the polymerase and may be adapted according to the supplier guidance.

Preferably, in these embodiments, the method further comprises, after lysis step when necessary,

contacting molecular targets with a labelling mixture comprising said DNA probes as described above, at least one DNA polymerase, a dNTP mix (dATP, dCTP, dGTP, dTTP) and optionally an appropriate buffer, and

converting RNA molecular targets into cDNA comprising a molecular identification DNA sequence (UMI) using polymerization reaction.

Alternatively, or additionally, at least some probes may be chimeric molecules made of synthetic DNA oligonucleotides, i.e. the DNA moiety, covalently associated to a second molecule, i.e. the capture moiety, targeting specifically and with high affinity the target molecule and making possible to specifically label any molecule with a signal amplifiable and readable. These probes may be specific of any type of molecular targets, preferably are specific of protein molecular targets.

The capture moiety of these probes may be

(i) a binding moiety that specifically binds to a molecular target and is directly bound to the DNA moiety,

(ii) a chimeric protein comprising a first domain that specifically binds to a molecular target and a second domain that binds to the DNA moiety, or

(iii) a binding moiety that binds specifically to a molecular target and a protein bridge, said protein bridge comprising a first domain that binds to the binding moiety and a second domain that binds to the DNA moiety.

The term “specifically binding” or “specifically binds” is used herein to indicate that this moiety has the capacity to recognize and interact specifically with the molecular target of interest, while having relatively little detectable reactivity with other structures present in the aqueous phase such as other molecular targets that can be recognized by other probes. There is commonly a low degree of affinity between any two molecules due to non-covalent forces such as electrostatic forces, hydrogen bonds, Van der Waals forces and hydrophobic forces, which is not restricted to a particular site on the molecules, and is largely independent of the identity of the molecules. This low degree of affinity can result in non-specific binding. By contrast when two molecules bind specifically, the degree of affinity is much greater than such non-specific binding interactions. In specific binding a particular site on each molecule interacts, the particular sites being structurally complementary, with the result that the capacity to form non-covalent bonds is increased. Specificity can be relatively determined by binding or competitive assays, using e.g., Biacore instruments. The affinity of a molecule X for its partner Y can generally be represented by the dissociation constant (Kd). In preferred embodiments, the Kd representing the affinity between the capture moiety and the molecular target of interest is from 1·10⁻⁷M or lower, preferably from 1·10⁻⁸M or lower, and even more preferably from 1·10⁻⁹M or lower.

In some embodiments, at least some probes comprise a capture moiety which is a binding moiety that specifically binds to a molecular target and is directly bound to the DNA moiety. Preferably the binding moiety is covalently bound to the DNA moiety.

Examples of binding moieties include, but are not limited to, antibodies, ligands of ligand/anti-ligand couples, peptide and nucleic acid aptamers, protein tags, or chemical probes (e.g. suicide substrate) reacting specifically with a molecular target or a class of molecular targets.

Examples of ligand/anti-ligand couples include, but are not limited to, antibody/antigen or ligand/receptor. In particular, in some embodiments, the molecular target is an antibody and the binding moiety is an antigen recognized by said antibody, or vice-versa. In some other embodiments, the molecular target is a receptor and the binding moiety is a ligand recognized by said receptor, or vice-versa.

A multitude of protein tags are well-known by the skilled person (see for example Young et al. Biotechnol. J. 2012, 7, 620-634) and may be used in the present invention. Examples of such protein tags include, but are not limited to, biotin (for binding to streptavidin or avidin derivatives), glutathione (for binding to proteins or other substances linked to glutathione-S-transferase), lectins (for binding to sugar moieties), c-myc tag, hemaglutinin antigen (HA) tag, thioredoxin tag, FLAG tag, polyArg tag, polyHis tag, Strep-tag, OmpA signal sequence tag, calmodulin-binding peptide, chitin-binding domain, cellulose-binding domain, S-tag, and Softag3, and the like.

A multitude of chemical probes are well-known by the skilled person (see for example Niphakis and Cravatt, Ann. Rev. of Biochem. 2014, 83, 341-77 and Willems et al. Bioconjugate Chem. 2014, 25, 1181-91) and may be used in the present invention. Examples of chemical probes include, but are not limited to, electrophile or photoreactive Activity-Based Probes (ABP), suicide substrate-based ABP and inhibitors-based ABP.

In a particular embodiment, at least some probes are aptamer-based probes, i.e. probes comprising a nucleic acid or peptide aptamer as capture moiety. Similar to antibodies, aptamers interact with their targets by recognizing a specific three-dimensional structure. Aptamers can specifically recognize a wide range of targets, such as proteins, nucleic acids, ions or small molecules such as drugs and toxins.

Peptides aptamers consist of a short variable peptide loop attached at both ends to a protein scaffold such as the bacterial protein thioredoxin-A. Typically, the variable loop length is composed of ten to twenty amino acids. Peptide aptamer specific of a target of interest may be selected using any method known by the skilled person such as the yeast two-hybrid system or Phage Display. Peptides aptamers may be produced by any method known by the skilled person such as chemical synthesis or production in a recombinant bacterium followed by purification.

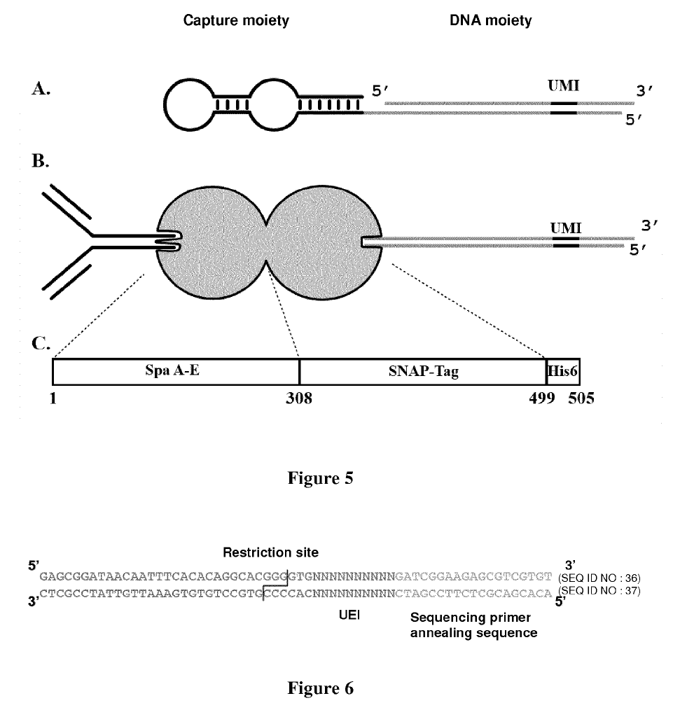

Preferably, at least some probes comprise a nucleic acid aptamer as capture moiety. Nucleic acid aptamers are a class of small nucleic acid ligands that are composed of RNA or single-stranded DNA oligonucleotides and have high specificity and affinity for their targets. Systematic Evolution of Ligands by EXponential enrichment (SELEX) technology to develop nucleic acid aptamers specific of a target of interest, is well known by the skilled person and may be used to obtain aptamers specific of a particular molecular target. Nucleic acid aptamers may be produced by any method known by the skilled person such as chemical synthesis or in vitro transcription for RNA aptamers. Preferably, nucleic acid aptamers used as capture moiety are selected from the group consisting of DNA aptamers, RNA aptamers, XNA aptamers (nucleic acid aptamer comprising xeno nucleotides) and spiegelmers (which are composed entirely of an unnatural L-ribonucleic acid backbone). An exemplary illustration of such a probe is presented in FIG. 5A.

In another particular embodiment, at least some probes are antibody-based probes, i.e. probes comprising an antibody as capture moiety.

The term “antibody” herein is used in the broadest sense and specifically covers monoclonal antibodies, polyclonal antibodies, antibody fragments, and derivatives thereof, so long as they specifically bind to the molecular target of interest. In particular, the antibody may be a full length monoclonal or polyclonal antibody, preferably a full length monoclonal antibody. Preferably, this term refers to an antibody with heavy chains that contain an Fc region. By “Fc”, “Fc fragment” or “Fc region”, used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. Preferably, the antibody is a full length monoclonal or polyclonal IgG antibody, preferably a full length monoclonal IgG antibody. A large number of specific and high affinity monoclonal antibodies are currently available on the market.

As used herein, the term “antibody fragment” refers to a protein comprising a portion of a full length antibody, generally the antigen binding or variable domain thereof. Examples of antibody fragments include Fab, Fab′, F(ab)₂, F(ab′)₂, F(ab)₃, Fv (typically the VL and VH domains of a single arm of an antibody), single-chain Fv (ScFv), dsFv, Fd (typically the VH and CH1 domains) and dAb (typically a VH domain) fragments, nanobodies, minibodies, diabodies, triabodies, tetrabodies, kappa bodies, linear antibodies, and other antibody fragments that retain antigen-binding function (e.g. Holliger and Hudson, Nat Biotechnol. 2005 September; 23(9):1126-36). Antibody fragments can be made by various techniques, including but not limited to proteolytic digestion of intact antibody as well as recombinant host cells (e.g. E. coli or phage). These techniques are well-known by the skilled person and are extensively described in the literature. Preferably, the antibody fragment is selected from the group consisting of Fab′, F(ab)₂, F(ab′)₂, F(ab)₃, Fv, single-chain Fv (ScFv) fragments and nanobodies.

The term “antibody derivative”, as used herein, refers to an antibody provided herein, e.g. a full-length antibody or a fragment of an antibody, wherein one or more of the amino acids are chemically modified, e.g. by alkylation, PEGylation, acylation, ester or amide formation or the like. In particular, this term may refer to an antibody provided herein that is further modified to contain additional nonproteinaceous moieties that are known in the art and readily available.

In some embodiments, the capture moiety is selected from the group consisting of monoclonal and polyclonal antibodies, Fab′, F(ab)₂, F(ab′)₂, F(ab)₃, Fv, single-chain Fv (ScFv) fragments and nanobodies, and derivatives thereof. In some preferred embodiments, the capture moiety is selected from the group consisting of a monoclonal antibody, a ScFv fragment or a nanobody.

In some embodiments, at least some probes comprise a capture moiety which is a chimeric protein comprising a first domain that specifically binds to a single molecular target and a second domain that binds to a single DNA moiety. Preferably the second domain is covalently bound to the DNA moiety.

The first domain of the chimeric protein specifically binds to a single molecular target. Examples of first domains include, but are not limited to, antibodies, ligands of ligand/anti-ligand couples, peptide and nucleic acid aptamers, protein tags, and chemical probes, as described above. Preferably, the first domain of the chimeric protein is selected from the group consisting of antibodies and peptide aptamers, more preferably is a monoclonal antibody.

In a particular embodiment, the first domain of the chimeric protein is an antibody, preferably selected from the group consisting of monoclonal and polyclonal antibodies, Fab′, F(ab)₂, F(ab′)₂, F(ab)₃, Fv, single-chain Fv (ScFv) fragments and nanobodies, and derivatives thereof. More preferably, the first domain of the chimeric protein is selected from the group consisting of a monoclonal antibody, a ScFv fragment or a nanobody, and even more preferably from the group consisting of a ScFv fragment or a nanobody.

The second domain that covalently binds to the DNA moiety may be any domain allowing covalently grafting of a single nucleic acid. Examples of such domains include, but are not limited to, SNAP-tag® (New England Biolabs), CLIP-tag® (New England Biolabs), Halo-tag® (Promega). Preferably, the second domain is a SNAP-tag®. The SNAP-tag is a 20 kDa mutant of the DNA repair protein O⁶-alkylguanine-DNA alkyltransferase that reacts specifically and rapidly with benzylguanine (BG) derivatives leading to irreversible covalent association of the SNAP-tag with the DNA moiety attached to BG.

The chimeric protein used as capture moiety and comprising the first and second domains may be produced as fusion protein using any well-known recombinant engineering technology, before to be covalently associated to the DNA moiety of the probe.

In some embodiments, at least some probes comprise a capture moiety comprising (i) a binding moiety that specifically binds to a molecular target and (ii) a protein bridge, said protein bridge comprising a first domain that binds to the binding moiety and a second domain that binds to the DNA moiety. Preferably the second domain is covalently bound to the DNA moiety. The first domain may be covalently or non-covalently bound to the binding moiety, preferably non-covalently bound. In embodiments wherein the first domain non-covalently binds to the binding moiety, the non-covalent interaction is preferably turned into covalent interaction by cross-linking the first domain and the binding moiety.

The second domain of the protein bridge may be as described above for the chimeric protein, i.e. any domain allowing covalently grafting of a single nucleic acid. Preferably, the second domain of the protein bridge is a SNAP-tag®.

The first domain of the protein bridge may be any domain allowing covalent or non-covalent interaction with the binding moiety, preferably non covalent interaction. Examples of such domain includes, but are not limited to, immunoglobulin-binding bacterial proteins such as protein A, protein A/G, protein G and protein L.

In an embodiment, the first domain of the protein bridge is an immunoglobulin-binding bacterial protein and the binding moiety is an antibody, preferably an antibody containing a Fc region, more preferably a full length monoclonal or polyclonal IgG antibody, preferably a full length monoclonal IgG antibody. The immunoglobulin-binding bacterial protein is preferably selected from protein A, protein A/G, protein G and protein L, one or several IgG-binding domains thereof, and functional derivatives thereof.

Protein A is a cell surface protein found in Staphylococcus aureus. It has the property of binding the Fc region of a mammalian antibody, in particular of IgG class antibodies. The amino-terminal region of this protein contains five highly homologous IgG-binding domains (termed E, D, A, B and C), and the carboxy terminal region anchors the protein to the cell wall and membrane. All five IgG-binding domains of protein A bind to IgG via the Fc region and in principle, each of these domains is sufficient for binding to the Fc-portion of an IgG.

Thus, in a particular embodiment, the first domain of the protein bridge is selected from the group consisting of domains A, B, C D and E of protein A, combinations thereof and functional derivatives thereof retaining IgG binding functionality of wild-type protein A. Preferably, the first domain of the protein bridge comprises domains A to E of protein A.

The protein bridge may be produced as fusion protein using any well-known recombinant engineering technology, before to be covalently associated to the DNA moiety of the probe.

Preferably, the first domain is located at the N-terminal part of the protein bridge and the second domain is located at the C-terminal part of the protein bridge.

Optionally, the protein bridge may further comprise at the C-terminal extremity an affinity tag (e.g. a polyhistidine-tag) to facilitate its purification.

In a particular embodiment, the protein bridge comprises an immunoglobulin-binding bacterial protein, preferably domains A to E of protein A, as first domain, a SNAP-tag® as second domain and a monoclonal or polyclonal IgG antibody as binding moiety, preferably a monoclonal IgG antibody. An exemplary illustration of such a probe is presented in FIGS. 5B and C.

The binding moiety may be covalently or non-covalently bound to the protein bridge. In some embodiments, the protein bridge can be cross-linked with the binding moiety to ensure long-term physical link.

In chimeric probes described above, the DNA moiety consists of a double stranded DNA molecule comprising an overhang, preferably compatible with a cohesive end generated by a restriction enzyme, or an overhang producing restriction site as described above for DNA probes. Preferably, the overhang comprised in the DNA moiety or generated by the restriction site is a 3′overhang. As described above, the DNA moiety comprises a unique molecular identification (UMI) sequence, and optionally a type identifier sequence.

Preferably, in chimeric probes, the DNA moiety further comprises a sequencing primer annealing sequence which is proximal to the capture moiety. After labelling of molecular targets, this sequence allows direct amplification using sequencing primers.

Preferably, in embodiments wherein at least some probes are chimeric probes, the method further comprises, after lysis step when necessary, contacting molecular targets with a labelling mixture comprising at least one chimeric probe.

The labelling mixture comprising probes, and optionally reagents to perform DNA polymerization reaction, may be added to the aqueous phase of the droplets before encapsulation of cells (i.e. by direct inclusion in the mixture or via a co-flow) or after droplet generation by any known technique such as pico-injection or droplet fusion. Preferably, the labelling mixture is added after incubation allowing cell lysis and/or elimination/degradation of some non-targeted molecules.

In a particular embodiment, the labelling mixture is encapsulated into w/o droplets and these droplets are fused with droplets comprising molecular targets. The content of each droplet comprising the labelling mixture should be substantially identical. Methods to produce such emulsion are well known by the skilled person as well as methods of generating, synchronizing and fusing emulsion droplets, in particular on a microfluidic device.

Providing the Second Set of Emulsion Droplets Comprising Cell Identification Sequences

The second set of emulsion droplets comprises droplets containing entity identification sequences, wherein each of these droplets contains at least one entity identification sequence.

The entity identification sequence is a double stranded DNA sequence of 40 to 100 nucleotide long, preferably of 50 to 70 nucleotide long, comprising a unique entity identification (UEI) barcode which is different for each droplet of the second set, and an overhang producing restriction site, preferably a 3′overhang producing restriction site.

The “UEI sequence” or “UEI barcode” is a randomized nucleotide sequence assigning the same barcode to each molecular target originating from the same entity. Preferably, the UEI sequence is a randomized nucleotide sequence having a length of at least 8 nucleotides, preferably a length from 8 to 20 nucleotides, more preferably a length from 8 to 15 nucleotides. The randomized sequence can be a stretch of contiguous randomized nucleotides or a stretch of semi-randomized nucleotides (i.e. contiguous randomized nucleotides spaced by constant nucleotides). Typical examples of a stretch of semi-randomized nucleotides are stretches where several randomized dinucleotides are spaced by constant dinucleotides, or stretches where several randomized trinucleotides are spaced by constant trinucleotides. Preferably, the UEI sequence is a stretch of semi-randomized nucleotides, in particular a stretch where several randomized dinucleotides are spaced by constant dinucleotides.

The restriction site comprised in the entity identification sequence may generate, upon digestion with the corresponding restriction enzyme, an overhang compatible with the overhangs of labelled molecular targets, or an overhang compatible with the overhangs of UEI calibrators as described below, and thus allows addition of UEI sequence to the molecular identification DNA sequence. Preferably, this restriction site is a non-palindromic cleavage site.

Optionally, the entity identification sequence may further comprise a sequencing primer annealing sequence adjacent to the UEI barcode and at the opposite end of the restriction site.

In preferred embodiments, the entity identification sequence comprises a constant region allowing for amplification of the UEI-bearing DNA, adjacent to the restriction site and at the opposite end of the UEI barcode and the sequencing primer annealing sequence when present. Preferably, this constant region has a length of 15 to 35 nucleotides, preferably of 20 to 30 nucleotides. Preferably, this constant region has a melting temperature comprised between 50° C. and 70° C.

An exemplary illustration of such entity identification sequence is presented in FIG. 6.

Preferably, all entity identification sequences have to same sequence except for the UEI barcode sequence, i.e. they exhibit the same sequencing primer annealing sequence, the same restriction site and the same constant region.

Whereas the UEI sequence has to be different for each droplet, it also has to be present in large number of identical copies in each droplet.

Thus, in an embodiment, the method further comprises encapsulating a plurality of entity identification sequences within emulsion droplets, each droplet containing no more than one entity identification sequence, with an amplification reaction mixture, and amplifying the entity identification sequences within droplets, thereby obtaining the second set of emulsion droplets. This way, more than one million of copies of the same entity identification sequence can be generated in each droplet and later serve to barcode the previously labeled molecular targets with an identical and unique UEI sequence. Entity identification sequences may be encapsulated in single or double stranded form, preferably in double stranded form.

The amplification reaction mixture comprises all reagents required to perform DNA amplification into the droplets, i.e. typically a DNA polymerase, primers, buffers, dNTPs, salts (e.g. MgCl2), etc. . . . . Primers are designed in order to allow the complete amplification of the entity identification sequence. In certain embodiments, amplification relies on alternating cycles of heating and cooling (i.e., thermal cycling) to achieve successive rounds of replication (e.g., PCR). Methods of amplifying genetic elements compartmentalized in emulsion droplets are well-know and widely practiced by the skilled person (see for example, Chang et al. Lab Chip. 2013 Apr. 7; 13(7):1225-42; Zanoli and Spoto, Biosensors 2013, 3, 18-43). In particular, the amplification may be performed by any known technique such as polymerase chain reaction (PCR), nucleic acid sequence-based amplification (NASBA), loop-mediated isothermal amplification (LAMP), helicase-dependent amplification (HDA), rolling circle amplification (RCA), multiple displacement amplification (MDA) and recombinase polymerase amplification (RPA). The suitable method can be easily chosen by the skilled person depending on the nature of the encapsulated genetic element and the molecular target. Preferably, entity identification sequences are amplified into the droplets by PCR amplification.

Preferably, in this embodiment, in order to prevent co-encapsulation of more than one entity identification sequence in one droplet, and thus to preserve single-entity resolution, the DNA solution comprising entity identification sequences is strongly diluted, e.g. in the amplification reaction mixture, such that, according to Poisson statistics driving molecule distribution, droplet occupancy, i.e. the percentage of droplets comprising a entity identification sequence, is limited to less than 20%, preferably to less than 10%. Thus, in particular embodiments, the method further comprises, before encapsulating entity identification sequences and amplification reaction mixture within emulsion droplets, diluting entity identification sequences, preferably into the amplification reaction mixture, in order to obtain a droplet occupancy of less than 20%, preferably less than 10%.

In order to increase droplet occupancy, encapsulation of several entity identification sequence in one droplet may be tolerated. In this case, the inventors found that single-entity resolution can be preserved by adding UEI-calibrator molecules to entity identification sequences. Thus, in a preferred embodiment, the method further comprises encapsulating a plurality of entity identification sequences within emulsion droplets in the presence of UEI-calibrators and an amplification reaction mixture, wherein at least some droplets comprise one or several entity identification sequences and one or several UEI-calibrators; and

amplifying entity identification sequences and/or UEI-calibrators within droplets, preferably entity identification sequences and UEI-calibrators using a multiplex reaction;

thereby obtaining the second set of emulsion droplets.

Entity identification sequences and UEI-calibrators may be encapsulated in single or double stranded form, preferably in double stranded form.

Preferably, for multiplex reaction, a higher amount of primers specific of the entity identification sequences is used relative to those specific for the UEI-calibrators.

The possibility of co-encapsulating several entity identification sequences in the same droplets leads to a dramatic increase of droplet occupancy. Indeed, adjusting the dilution of the entity identification sequence solution in order to co-encapsulate five entity identification sequences per droplets, leads to a droplet occupancy close to 100%.

Preferably, dilutions of entity identification sequence solution and UEI-calibrator solution are adjusted in order to co-encapsulate 2 to 10 entity identification sequences per droplet, preferably 4 to 6 entity identification sequences per droplet, and 2 to 10 UEI-calibrators per droplet, preferably 4 to 6 UEI-calibrators per droplet.

UEI-calibrators are DNA sequences, preferably double stranded DNA sequences, comprising a unique calibrator barcode which is different for each UEI-calibrator and for each droplet, and one or two overhang producing restriction sites, preferably generating overhangs compatible with overhangs of digested entity identification sequences and/or labelled molecular targets.

Preferably, these overhang producing restriction sites are non-palindromic cleavage sites. Preferably, these restriction sites generate 3′ overhangs. Preferably, these restriction sites are different from the restriction site generating compatible overhangs on entity identification sequences and/or the restriction site generating compatible overhangs on labelled molecular targets. The “UEI-calibrator sequence” or “UEI-calibrator barcode” is a randomized nucleotide sequence having a length of at least 15 nucleotides, preferably a length from 15 to 40 nucleotides, more preferably a length from 15 to 20 nucleotides. The randomized sequence can be a stretch of contiguous randomized nucleotides or a stretch of semi-randomized nucleotides (i.e. contiguous randomized nucleotides spaced by constant nucleotides). Typical examples of a stretch of semi-randomized nucleotides are stretches where several randomized dinucleotides are spaced by constant dinucleotides, or stretches where several randomized trinucleotides are spaced by constant trinucleotides. Preferably, the UEI-calibrator barcode is a stretch of semi-randomized nucleotides, in particular a stretch where several randomized dinucleotides are spaced by constant dinucleotides.

In an embodiment, UEI-calibrators comprise an overhang producing restriction site which generates, upon digestion with the corresponding restriction enzyme, an overhang compatible with overhangs of digested entity identification sequences comprising UEI barcodes. In this embodiment, since both types of molecules, i.e. UEI-calibrator sequences and entity identification sequences, carry restriction sites generating compatible extremities, the subsequent digestion/ligation step used for UEI addition to labelled molecular targets, leads to the formation of different chimeras between UEI and UEI-calibrator barcodes. Indeed, whereas a large fraction of UEI barcodes will stay available for the barcoding of the labeled molecular targets originating from the entity, a sub fraction of UEI barcodes will get associated to each of the UEI-calibrator barcodes. These pairs of sequences constitute a signature unique to each droplet and allow reassigning each analyzed molecule to its original entity, despite the presence of multiple entity identification sequences in some droplets (see e.g. FIG. 7). Optionally, in this embodiment, UEI-calibrators may further comprise a sequencing primer annealing sequence adjacent to the UEI-calibrator barcode and at the opposite end of the restriction site generating overhangs compatible with digested entity identification sequences. Optionally, UEI-calibrators may further comprise a binding tag allowing specific capture of the molecule at the extremity proximal to the sequencing primer annealing sequence. Preferably, the binding tag is selected from biotin or digoxigenin, more preferably is biotin.

Preferably, in this embodiment, UEI-calibrators comprise a constant region adjacent to the restriction site and at the opposite end of the UEI-calibrator barcode and the sequencing primer annealing sequence when present. This constant region allows amplifying UEI-calibrators, preferably using PCR amplification. In particular, this region may comprise a primer binding site. Preferably, this constant region has a length of 10 to 35 nucleotides, preferably of 15 to 25 nucleotides. Preferably this constant region is orthogonal to that of entity identification sequences in order to prevent unwilling hybridization. UEI-calibrators may further comprise an additional region comprised between the UEI-calibrator barcode and the sequencing primer annealing sequence when present. This region acts as a spacer between the sequencing primer annealing sequence and the UEI-calibrator barcode and may be used to adjust the length of the amplified sequences comprising UEI and UEI-calibrator barcodes to the length of the amplified sequences comprising UMI and UEI barcodes to limit potent amplification biases. An exemplary illustration of such UEI-calibrator is presented in FIG. 9.

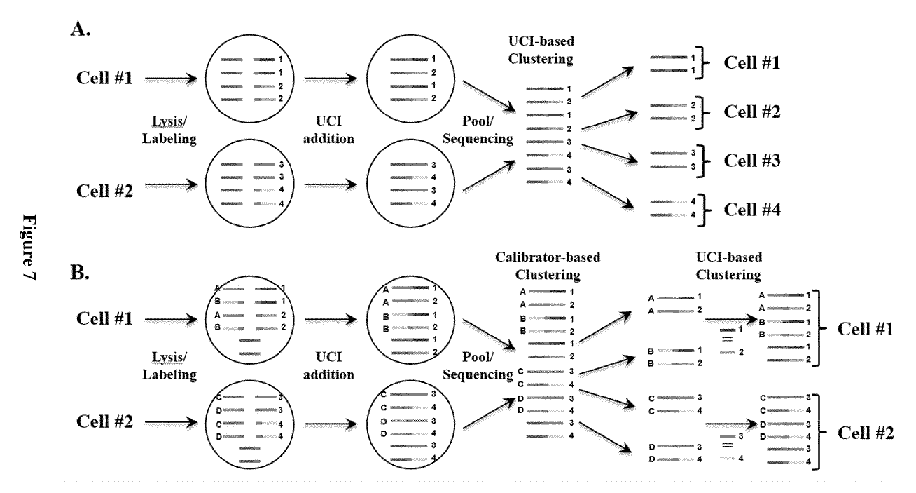

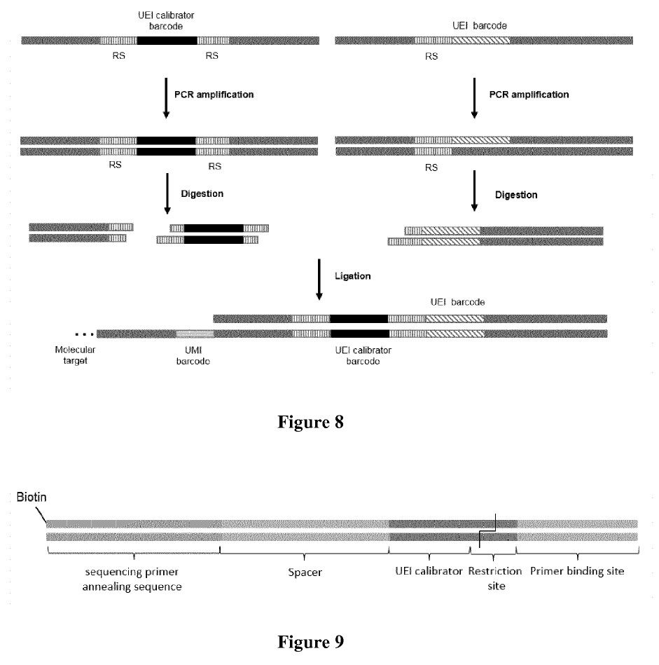

In another embodiment, UEI-calibrators comprise two overhang producing restriction sites, a first restriction site generating an overhang compatible with overhangs of digested entity identification sequences comprising UEI barcodes and a second restriction site generating an overhang compatible with overhangs of labelled molecular targets. In this embodiment, the subsequent digestion/ligation step used for UEI addition to labelled molecular targets, leads to the formation of tripartite molecules comprising a labelled molecular target, an UEI sequence and a UEI-calibrator barcode (see e.g. FIG. 8).

In this embodiment, UEI-calibrators may comprise constant regions adjacent to each of the two overhang producing restriction sites. These constant regions allow amplifying UEI-calibrators, preferably using PCR amplification. In particular, these regions may comprise primer binding sites. Preferably, these constant regions have a length of 10 to 35 nucleotides, preferably of 15 to 25 nucleotides. Preferably these constant regions are orthogonal to that of entity identification sequences in order to prevent unwilling hybridization. An exemplary illustration of such UEI-calibrator is presented in FIG. 9.

Preferably, all UEI-calibrators have to same sequence except for the UEI-calibrator barcode sequence, i.e. they exhibit, the same restriction site, the same constant region(s) and optionally the same sequencing primer annealing sequence.

As described above, multiplex amplification of entity identification sequences and UEI-calibrators within droplets may be performed using any method known by the skilled person, preferably multiplex PCR. The amplification reaction mixture comprises all reagents required to perform DNA amplification into the droplets, i.e. typically a DNA polymerase, primers, buffers, dNTPs, salts (e.g. MgCl2), etc. . . . and primers are designed in order to allow the complete amplification of entity identification sequences and UEI calibrators.

Preferably, in embodiments producing chimeras between UEI and UEI-calibrator barcodes, in order to ensure a large over-representation of entity identification sequences and allow the proper barcoding of molecular targets after droplet fusion, the amplification reaction mixture comprises a higher amount of primers specific of the entity identification sequences relative to those specific for the UEI-calibrators. More preferably, the amplification reaction mixture comprises at least 5 times, preferably at least 10 times, and more preferably at least 100 times, higher amount of primers specific of the cell identification sequences relative to those specific for the UEI-calibrators.

In embodiments producing tripartite molecules comprising a labelled molecular target, an UEI sequence and a UEI-calibrator barcode, the amplification reaction mixture may comprise similar amounts of primers specific of the entity identification sequences and primers specific for the UEI-calibrators.

Optionally, UEI sequence and UEI-calibrators may be assembled through their compatible overhangs before amplification. In this case, amplification reaction therefore directly amplify a fragment comprising UEI sequence and UEI-calibrators. After fusion with the first set of droplets, said fragment is ligated to labelled molecular targets.

Droplet Fusion and Incorporation of UEI Barcodes, and Optionally UEI-Calibrator Barcodes, into Labelled Molecular Targets

As described above, the first set of emulsion droplets comprises molecular targets labelled with molecular identification sequence comprising UMI barcodes, and the second set of emulsion droplets comprises entity identification sequences comprising UEI barcodes, and optionally UEI calibrators.

The method of the invention comprises fusing droplets of the first set with droplets of the second set wherein a droplet of the first set is fused with no more than one droplet of the second set.

Any technique known by the skilled person may be used to fuse a first droplet and a second droplet together to create a combined droplet. For example, opposite electric charges may be given to the first and second droplets (i.e., positive and negative charges, not necessarily of the same magnitude), which may increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc.

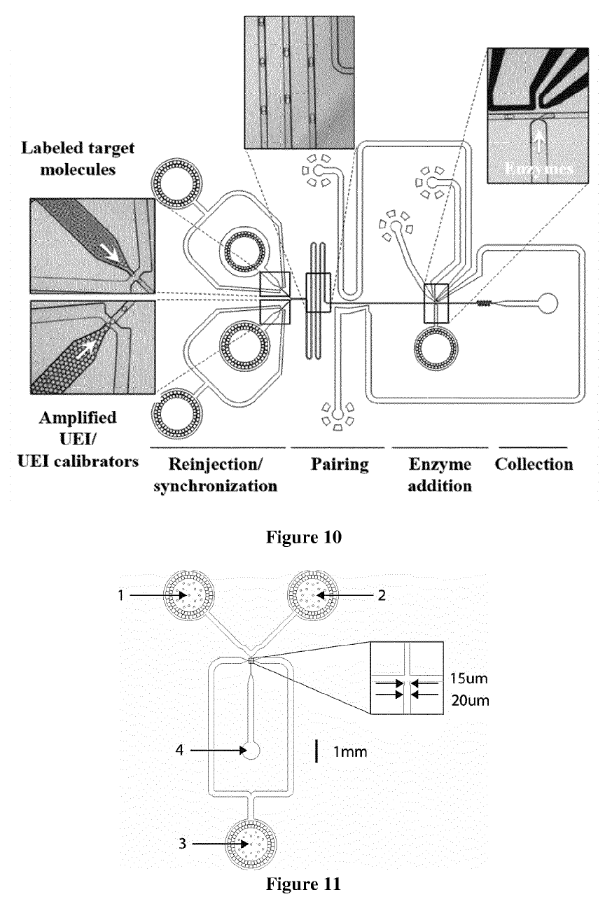

Any technique known by the skilled person may be used to ensure that a droplet of the first set is fused with no more than one droplet of the second set. In particular, droplets may be paired through a pairing channel before to reach the coalescence point. The use of such channel is well-known by the skilled person in order to control fusion of microfluidic droplets. In embodiments wherein a pairing channel is used, droplets of the first and second sets should have different sizes. The pairing channel is a long channel having a width larger than the smallest droplets and narrower than the largest droplets. As a consequence, the small droplet catches the large one and pairs of droplets are formed at the exit of the channel. This configuration ensures that droplets properly pairwise prior to reaching the coalescence point. An exemplary embodiment of such pairing channel is illustrated in FIG. 10.

In some embodiments, droplets of the first set and/or droplets of the second sets are generated on separate microfluidic system(s) and re-injected into the device. Preferably, droplets are spaced with oil streams and synchronized before to enter the pairing channel.

Preferably at least 60%, more preferably at least 80% of droplets, and even more preferably at least 95% of the first set are fused with a droplet of the second set.

After droplet fusion, UEI barcodes are incorporated into labelled molecular targets through restriction enzyme digestion and ligation.

In embodiments wherein droplets of the second set do not comprise UEI calibrators, UEI barcodes are incorporated into labelled molecular targets through restriction enzyme digestion and ligation of the compatible overhangs of labelled molecular targets and digested entity identification sequences comprising UEI barcodes.

In embodiments wherein droplets of the second set comprise UEI calibrators and said UEI calibrators comprise a overhang producing restriction site, (i) UEI calibrator barcodes and UEI barcodes on one hand, and (ii) UEI barcodes and labelled molecular targets on the other hand, may be assembled through restriction enzyme digestion and ligation of compatible overhangs of (i) UEI calibrators and entity identification sequences and of (ii) entity identification sequences and labelled molecular targets. In this embodiment, entity identification sequences are appended to both the labelled molecular targets and the UEI-calibrators through a digestion/ligation coupled reaction. An exemplary illustration of such embodiment is presented in FIG. 7B. Alternatively, UEI calibrator barcodes, UEI barcodes and labelled molecular targets are assembled through restriction enzyme digestion and ligation of compatible overhangs of i) UEI calibrators and entity identification sequences and (ii) entity identification sequences and labelled molecular targets.

In embodiments wherein droplets of the second set comprise UEI calibrators and said UEI calibrators comprise two overhang producing restriction sites, UEI calibrator barcodes, UEI barcodes and labelled molecular targets are assembled to through restriction enzyme digestion and ligation of compatible overhangs of (i) UEI calibrators and labelled molecular targets on one hand and (ii) UEI calibrators and entity identification sequences on the other hand. In this embodiment, the entity identification sequence, the UEI-calibrator and the labelled molecular target together form a tripartite molecule. An exemplary illustration of such embodiment is presented in FIG. 8.

In an embodiment, restriction sites comprised on entity identification sequences and UEI calibrators are identical and only one restriction enzyme is used to digest entity identification sequences and UEI calibrators. In embodiments wherein molecular identification DNA sequences comprise a restriction site generating an overhang, this restriction site and restriction sites comprised on entity identification sequences and UEI calibrators may be identical or different, preferably different.

In another embodiment, restriction sites comprised on entity identification sequences and UEI calibrators are different and two different restriction enzymes are used to digest entity identification sequences and UEI calibrators. The use of different restriction sites and of labels with compatible extremities ensures that a productive ligation event leads to the destruction of the restriction site. Therefore, the resulting chimeric molecule will not be a substrate of the restriction enzymes present in the mixture and the equilibrium is pulled toward the formation of the wished ligation products. In embodiments wherein molecular identification DNA sequences comprise a restriction site generating an overhang, this restriction site may be identical or different of the restriction site comprised on entity identification sequences or on UEI calibrators. Preferably, this restriction site is identical to the restriction site comprised on UEI calibrators.

Preferably, these restriction sites are non-palindromic in order to ensure directionality of the association of cell identification sequences, UEI calibrators and labelled molecular targets.

In embodiments wherein UEI sequence and UEI-calibrators are assembled through their compatible overhangs before amplification, i.e. before fusion of the two sets of droplets, the fragment comprising UEI sequence and UEI-calibrators can be ligated to labelled molecular targets through compatible overhangs of i) UEI calibrators and labelled molecular targets or (ii) entity identification sequences and labelled molecular targets.

Restriction enzymes, DNA ligase and optionally buffer may be added to the droplets after droplet fusion, e.g. by pico-injection or droplet fusion, or concomitantly to droplet fusion, e.g. by injection at a T-junction (see e.g. FIG. 10). Injection may be performed at the coalescence point where the presence of electrodes triggers both droplet fusion and enzyme delivery.

The emulsion may be then collected and incubated to allow digestion and ligation and thus association of UEI barcodes and labelled molecular targets, and UEI-calibrator barcodes when present.

In a particular embodiment wherein the molecular target is a nucleic acid, preferably a RNA, a sequencing primer annealing sequence may be added to labelled molecular target after incorporating UEI barcodes, and optionally UEI-calibrator barcodes. In particular, the method may further comprise, after incorporating UEI barcodes, and optionally UEI-calibrator barcodes, performing primer extension reaction using primers comprising from their 3′end to their 5′end, a region that hybridizes to complementary strands of molecular targets, i.e. to cDNA, and a sequencing primer annealing sequence.

Optionally, at their 5′ extremity, the primers may further comprise a binding tag allowing the specific capture of primer extension reaction products. Preferably, the binding tag is biotin or digoxigenin, more preferably is biotin.

Primer extension reaction may be performed as described above and reaction mixture may be brought into the droplet using any known method such as pico-injection or droplet fusion. Alternatively, the primer extension reaction can be performed in bulk upon droplet breaking and content recovery.

After completion of the labelling process, droplets may be broken and their content may be recovered, i.e. droplet lysate, e.g. to be further analysed/sequenced.

Analysis of Molecular Targets Labelled with the Method of the Invention

In a further aspect, the present invention also relates to a method of quantifying one or several molecular targets from a plurality of entities with single-entity resolution, said method comprising

labelling said molecular targets according to the method of the invention as described above, capturing said labelled molecular targets comprising UMI, UEI, and optionally UEI calibrator barcodes,

amplifying sequences comprising UMI and UEI barcodes, and optionally UEI-calibrator barcodes

sequencing said amplified sequences.

All embodiments described above for the method of the invention are also encompassed in this aspect.

The step of capturing labelled molecular target comprising UMI, UEI, and optionally UEI calibrator barcodes, allows removing from the droplet lysate untargeted molecules, unreacted entity identification sequences and/or UEI calibrators when present. This step may be performed using any capture molecule which is able to specifically bind molecular targets such as an antibody or nucleic acid specific of a molecular target, attached to a support. The capture molecule may be directly or indirectly attached to the support. In particular, the capture molecule may comprise a binding tag, e.g. biotin, interacting with a partner, e.g. streptavidin, linked to the support. The capture molecule may be for example a biotinylated monoclonal antibody specific of a targeted protein, or synthetic DNA oligonucleotide specific of a cDNA produced from a targeted RNA. A binding tag can be added during the synthesis of the second cDNA strand by primer extension mentioned above. The support may be chosen in order to allow washing of unreacted molecules. Preferably the support is beads, more preferably streptavidin conjugated beads. In some preferred embodiments, the support is magnetic beads, in particular streptavidin conjugated magnetic beads.

The nucleic acids comprising (i) the molecular identification DNA sequence comprising the UMI barcode and optionally the type identifier and (ii) the entity identification sequence comprising the UEI barcode are then sequenced using any method known by the skilled person, preferably using a next generation sequencing method.

In some embodiments wherein the entity identification sequence, the UEI-calibrator and the labelled molecular target together form a tripartite molecule, the nucleic acids to be sequenced comprise i) the molecular identification DNA sequence comprising the UMI barcode and optionally the type identifier, (ii) the entity identification sequence comprising the UEI barcode and (iii) the UEI-calibrator barcode.

In some other embodiments wherein the entity identification sequences are separately associated with UEI-calibrators and with molecular identification DNA sequence, the nucleic acids to be sequenced comprise (i) the molecular identification DNA sequence comprising the UMI barcode and optionally the type identifier and the entity identification sequence comprising the UEI barcode, and (ii) the entity identification sequence comprising the UEI barcode and the UEI-calibrator barcode.

In some embodiments, wherein said nucleic acids do not comprise any sequencing primer annealing sequences, said sequences may be added before the sequencing step, preferably by DNA amplification using primers comprising said sequences or by ligation of oligonucleotides comprising said sequences.

Preferably, when the molecular target is RNA, a part of this RNA is also sequenced.

One of the main advantage of the present invention is the possibility of pooling all captured labelled molecular targets prior to the sequencing step, whatever the type of molecular targets (e.g. RNA or protein). Sequences may be then analyzed using any method known by the skilled person such as bioinformatics to cluster said sequences and determining the absolute quantification of molecular targets. Firstly, UEI and UEI-calibrator barcodes are used to cluster the set of UEI labeling the molecules originating from the same entity. Secondly, UEI barcodes are used to cluster molecules according to their entity of origin. Third, molecules are clustered according to their type and identity, optionally using the type Identifiers, and, finally, redundant sequences are eliminated using the UMI barcodes giving access to the absolute quantification of each molecule with single-entity resolution.

Microfluidic Devices

In a further aspect, the present invention also relates to a microfluidic device suitable for implementing at least one step of the methods of the invention.

All embodiments described above for the methods of the invention are also encompassed in this aspect.

As used herein, the term “microfluidic device”, “microfluidic chip” or “microfluidic system” refers to a device, apparatus or system including at least one microfluidic channel.

The microfluidic system may be or comprise silicon-based chips and may be fabricated using a variety of techniques, including, but not limited to, hot embossing, molding of elastomers, injection molding, LIGA, soft lithography, silicon fabrication and related thin film processing techniques. Suitable materials for fabricating a microfluidic device include, but are not limited to, cyclic olefin copolymer (COC), polycarbonate, poly(dimethylsiloxane) (PDMS), poly(methyl methacrylate) (PMMA), and glass. Preferably, microfluidic devices are prepared by standard soft lithography techniques in PDMS and subsequent bonding to glass microscope slides. Due to the hydrophilic or hydrophobic nature of some materials, such as glass, which adsorbs some proteins and may inhibit certain biological processes, a passivating agent may be necessary. Suitable passivating agents are known in the art and include, but are not limited to silanes, fluorosilanes, parylene, n-dodecyl-β-D-maltoside (DDM), poloxamers such as Pluronics.

As used herein, the term “channel” refers to a feature on or in an article (e.g., a substrate) that at least partially directs the flow of a fluid. The term “microfluidic channel” refers to a channel having a cross-sectional dimension of less than 1 mm, typically less than 500 μm, 200 μm, 150 μm, 100 μm or 50 μm, and a ratio of length to largest cross-sectional dimension of at least 2:1, more typically at least 3:1, 5:1, 10:1 or more. It should be noted that the terms “microfluidic channel”, microchannel” and “channel” are used interchangeably in this description. The channel can have any cross-sectional shape (circular, oval, triangular, irregular, square or rectangular, or the like). Preferably, the channel has a square or rectangular cross-sectional shape. The channel can be, partially or entirely, covered or uncovered. As used herein, the term “cross-sectional dimension” of a channel is measured perpendicular to the direction of fluid flow.

The microfluidic device of the invention comprises
a first emulsion re-injection module or on-chip droplet generation module;
a second emulsion re-injection module or on-chip droplet generation module
a droplet-pairing module, and
a module coupling droplet fusion to injection,

wherein emulsion re-injection modules and/or on-chip droplet generation modules are in fluid communication and upstream to the droplet-pairing module, the droplet-pairing module is in fluid communication and upstream to the module coupling droplet fusion to injection.

As used herein, the term “upstream” refers to components or modules in the direction opposite to the flow of fluids from a given reference point in a microfluidic system.

As used herein, the term “downstream” refers to components or modules in the direction of the flow of fluids from a given reference point in a microfluidic system.

In an embodiment, the microfluidic device of the invention comprises

two emulsion re-injection modules

a droplet-pairing module, and

a module coupling droplet fusion to injection.

An exemplary embodiment of such microfluidic device is presented in FIG. 10.

In another embodiment, the microfluidic device of the invention comprises

an emulsion re-injection module and an on-chip droplet generation module,

a droplet-pairing module, and

a module coupling droplet fusion to injection.

In a further embodiment, the microfluidic device of the invention comprises

two on-chip droplet generation modules,

a droplet-pairing module, and

a module coupling droplet fusion to injection.

The emulsion re-injection module may be easily designed by the skilled person based on any known techniques. Typically, an emulsion re-injection module comprises a v-shaped structure where injected droplets are spaced by carrier oil supplying by at least one, preferably two side channels connected with the re-injection channel.

The module for generating droplets may be easily designed by the skilled person based on any known techniques. For example, emulsion droplets may be produced in the droplet generation module by any technique known by the skilled person such as drop-breakoff in co-flowing streams, cross-flowing streams in a T-shaped junction (see for example WO 2002/068104), and hydrodynamic flow-focussing (reviewed by Christopher and Anna, 2007, J. Phys. D: Appl. Phys. 40, R319-R336).

As explained above, the droplet-pairing module is a channel with dimensions allowing the contact between droplets of the two sets. In an embodiment, the width of the channel is about the diameter of the larger droplets and the depth of the channel is lower than the diameter of the larger droplets. In another embodiment, the depth of the channel is about the diameter of the larger droplets and the width of the channel is lower than the diameter of the larger droplets.

The length of the pairing channel has to be sufficient to obtain a contact between droplets of the first and second sets. Preferably, the time of contact is greater than 1 ms, preferably greater than 4 ms. As used herein, “the contact time τ” refers to the time in which paired droplets stay in physical contact before reaching the end of the pairing channel. Typically, the length of the pairing channel is ranging from 100 μm to 10 mm, preferably from 500 μm to 2 mm, and more preferably is about 1.5 mm.

The module coupling droplet fusion to injection is preferably a module wherein droplets, after pairing, are exposed to an electric field destabilizing their interface thanks to the proximity of electrodes, and are, in the same time, contacted with a stream injected in the channel. Destabilization of the interface then leads not only to droplet coalescence but also to infusion of the injected stream into droplets.

The microfluidic device may further comprise a collection module wherein fused droplets are recovered.

Optionally, the microfluidic device of the invention may comprise an inlet downstream to the module coupling droplet fusion to injection and upstream to the collection module. This inlet may be used to inject additional surfactant in order to increase the stability of fused droplets and to prevent any coalescence during the storage.

In another aspect, the present invention also relates to a kit comprising a microfluidic device according to the invention and as described above.

The kit may further comprise one or several microfluidic chips comprising an on-chip droplet generation module.

The kit of the invention may further comprise
one or several probes as described above; and/or
one or several entity identification sequences as described above; and/or
one or several UEI-calibrators as described above; and/or
one or several primers suitable to amplify entity identification sequences and/or UEI-calibrators; and/or
an aqueous phase and/or an oil phase; and/or
a leaflet providing guidelines to use such a kit.

All embodiments described above for the methods and the microfluidic device of the invention are also encompassed in this aspect.

In another aspect, the present invention also to a kit comprising
one or several probes as described above; and/or
one or several entity identification sequences as described above; and/or
one or several UEI-calibrators as described above; and/or
one or several primers suitable to amplify entity identification sequences and/or UEI-calibrators; and/or
an aqueous phase and/or an oil phase; and/or
one or several microfluidic devices, in particular one or several microfluidic devices as described above, and optionally
a leaflet providing guidelines to use such a kit.

All embodiments described above for the methods and the microfluidic device of the invention are also encompassed in this aspect.

The present invention further relates to the use of a kit of the invention to label a plurality of molecular targets from a plurality of entities according to the method of the invention, or to quantify one or several molecular targets from a plurality of entities according to the method of the invention. All embodiments described above for the methods, the microfluidic device and the kit of the invention are also encompassed in this aspect.

As used herein, the verb “to comprise” is used in its non-limiting sense to mean that items following the word are included, but items not specifically mentioned are not excluded.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “about” refers to a range of values ±10% of the specified value. For example, “about 20” includes ±10% of 20, or from 18 to 22. Preferably, the term “about” refers to a range of values ±5% of the specified value.

All patent and literature references cited in the present specification are hereby incorporated by reference in their entirety.

### EXAMPLES

I. Microfluidics Chips Fabrication and Operation

In all the examples presented below microfluidic chips were fabricated using the same procedure and they were manipulated using on the same workstation.

a. Microfluidic Chips Preparation and Operation

Microfluidic devices were obtained using a classic replica molding process as described previously in (Mazutis et al., 2009). Briefly, devices were designed on Autocad (Autodesk 2014), negative photomasks were printed (Selba S. A.) and used to prepare molds by standard photolithography methods. SU8-2010 and SU8-2025 photoresist (MicroChem Corp.) were used to pattern 10 to 40 μm deep channels onto silicon wafers (Siltronix). Microfluidic devices were then fabricated in polydimethylsiloxane (PDMS, Silgard 184, Dow-Corning) using conventional soft lithography methods (Xia and Whitesides, 1998). Upon plasma bonding to a glass slide, channels were passivated with a solution of 1% (v/v) 1H, 1H, 2H, 2H-perfluorodecyltrichlorosilane (97%, ABCR GmbH and Co) in HFE7500 (3M) and subsequently flushed with compressed air. Key dimensions and depth of microfluidic devices are given on concerned figures and in their captions.

Aqueous phases were loaded in I.D. 0.75 mm PTFE tubings (Thermo Scientifc) and oils were loaded in 2 mL Micrew Tubes (Thermo Scientific). Liquids were injected into microfluidic devices at constant and highly controlled flow-rates using a 7-bar MFCS™ pressure-driven flow controller (Fluigent) equipped with Flowells (7 μL/min flow-meters) allowing for operation in flow-rate controlled mode.

b. Optical Set-Up, Data Acquisition and Control System

The optical setup was based on an inverted microscope (Nikon Eclipse Ti-S) mounted on a vibration-dampening platform (Thorlabs B75150AE). The beams of a 488 nm laser (CrystaLaser DL488-050-0) and a 561 nm laser (Cobolt DPL 561-NM-100MW) were combined using a dichroic mirror (Semrock 2F495-DI03-2536). They were further combined with a 375 nm laser (CrystaLaser DL375-020-0) using a second dichroic mirror (Semrock Di02-R405-25×36) prior to shaping the resulting combined beam as lines using a pair of lenses (Semrock LJ1878L2-A and 111567L1-A) that was directed into the microscope objective (Nikon Super Plan Fluor 20× ELWD or Nikon Super Plan Fluor 40× ELWD) to be focused in the middle of the channel at the detection point. The emitted fluorescence was collected by the same objective and separated from the laser beams by a multi-edges dichroic mirror (Semrock Di01-R405/488/561/635-25×36). Blue (7-amino coumarin-4-methanesulfonic acid) fluorescence was resolved from green (Syto9, FAM) and orange (Texas-Red) fluorescence by a third dichroic mirror (Semrock LM01-480-25). Then green fluorescence was separated from orange fluorescence by an additional dichroic mirror (Semrock FF562-Di03-25×36). Fluorescence was finally measured by three photomultiplier tubes (Hamamatsu H10722-20) equipped with bandpass filters (Semrock FF01-445/45-25, FF01-600/37-25 and FF03-525/50-25 for blue, green and orange detection respectively). Signal acquisition from the PMTs was performed using an intelligent data acquisition (DAQ) module featuring a user-programmable FPGA chip (National Instruments PCI-7851R) driven by internally developed firmware and software. To monitor the experiment, we used an additional dichroic mirror (Semrock FF665-Di02-25×36) to split light to a CCD camera (Allied Vision Technologies Guppy F-033). A long-pass filter (Semrock BLP01-664R-25) prevented potentially damaging reflections of the lasers into the camera.

II. Sequences Used in the Examples

TABLE 1
Sequences used in the examples

| | SEQ | |
|---|---|---|
| Molecule | ID no | 5′-Sequence-3' |
| Template | 1 | GAGCGGATAACAATTTCACACAGGCACGGGGTGTGAGATCACAGA |
| UEI-DraIII | | TCGGAAGAGCGTCGTGT |
| UEI-Fwd | 2 | GAGCGGATAACAATTTCACACAGG |
| UEI-Rev | 3 | ACACGACGCTCTTCCGATC |
| Template | 4 | TCGTCGGCAGCGTCAGATGTGTATAAGAGACAGGGAAGACGTAGCA |
| Calibrator-N15- | | AGGGAGTAGCCAATGTGAATTGAGAGCCTTAAGCTGTATNNNNNNN |
| AlwNI | | NNNNNNNNNNNNNNNNNNCAGGGGCTGGTCGTGACTGGGAAAAC |
| | | CCTGGC |
| Calib-Rev | 5 | GCCAGGGTTTTCCCAGTCACGAC |
| Illu-2-Fwd | 6 | TCGTCGGCAGCGTCAGATG |
| Template UEI- | 7 | GAGCGGATAACAATTTCACACAGGCACGGGGTGNNNNNNNNNNNN |
| N15-DraIII* | | NNNGATCGGAAGAGCGTCGTGT |
| Template | 8 | TCGTCGGCAGCGTCAGATGTGTATAAGAGACAGGGAAGACGTAGCA |
| Calibrator-N15- | | AGGGAGTAGCCAATGTGAATTGAGAGCCTTAAGCTGTATCAGGACC |
| AlwNI-1g | | AGAGAGATGANNNNNNNNNNNNNNNCAGGGGCTGGTCGTGACTG |
| | | GGAAAACCCTGGC |
| Illu1 rev UEI | 9 | GTCTCGTGGGCTCGGAGATGTGTATAAGAGACAGACACGACGCTCT |
| | | TCCGATC |
| Illu1 | 10 | GTCTCGTGGGCTCGGAGAT |
| Template-AlwNI- | 11 | GGAAGACGTAGCAAGGGAGTAGCCAATGTGAATTCACACAGTGNN |
| N15-Calib-DraIII | | NNNNNNNNNNNNNCAGGGGCTGGTCGTGACTGGGAAAACCCTGGC |
| Calib-Fwd | 12 | GGAAGACGTAGCAAGGGAGTAGCC |
| RTmimicks | 13 | [phosphate]CTGCGCGGATCCCGGAAGCGAGGCCAGCTGGCTGCNNNN |
| | | NNNNTTGGCTGTCTCTTATACACATCTGACGCTGCCGACGA |
| antiSBACA- | 14 | GCAGCCAGCTGGCCTCGCTTCCGGGATCCGCGCAGACA |
| AlwN1 | | |
| Template UEI- | 15 | GAGCGGATAACAATTTCACACAGGCACGGGGTGNNNNNNNNNNNN |
| N15-DraIII-Illu1 | | NNNGATCGGAAGAGCGTCGTGTCTGTCTCTTATACACATCTCCGAGC |
| | | CCACGAGAC |
| RNA III | 16 | AUUAAUACGACUCACUAUACCUAGAUCACAGAGAUGUGAUGGAAA |
| | | AUAGUUGAUGAGUUGUUUAAUUUUAAGAAUUUUUAUCUUAAUUA |
| | | AGGAAGGAGUGAUUUCAAUGGCACAAGAUAUCAUUUCAACAAUC |
| | | GGUGACUUAGUAAAAUGGAUUAUCGACACAGUGAACAAAUUCAC |
| | | UAAAAAAUAAGAUGAAUAAUUAAUUACUUUCAUUGUAAAUUUGU |
| | | UAUCUACGUAUAGUACUAAAAGUAUGAGUUAUUAAGCCAUCCCAA |
| | | CUUAAUAACCAUGUAAAAUUAGCAAGUGAGUAACAUUUGCUAGU |
| | | AGAGUUAGUUUCCUUGGACUCAGUGCUAUGUAUUUUUCUUAAUU |
| | | AUCAUUACAGAUAAUUAUUUCUAGCAUGUAAGCUAUCGUAAACA |
| | | ACAUCGAUUUAUCAUUAUUUGAUAAAUAAAAUUUUUUUCAUAAU |
| | | UAAUAACAUCCCCAAAAAUAGAUUGAAAAAAUAACUGUAAAACA |
| | | UUCCCUUAAUAAUAAGUAUGGUCGUGAGCCCCUCCCAAGCUCGCG |
| | | GCCUUUUG |
| RT-UMI- | 18 | [phosphate]CTGCGCGGATCCCGGAAGCGAGGCCAGCTGGCTGCNNNN |
| RNAIII* | | NNTTGGGAGGGGCTCA |
| SB-alone | 20 | CTGCGCGGATCCCGGAAGCGAGGCCAGCTGGCTGC |
| RNAIII-Fwd | 21 | TCGTCGGCAGCGTCAGATGTGTATAAGAGACAGATAACTGTAAAAC |
| | | ATTCCCTTAATAATAAG |
| Gfp mRNA | 22 | AUGACCAGCUACCCAUACGAUGUUCCAGAUUACGCUAUCGAAGGC |
| | | CGCGGCCGCCAUCAUCAUCAUCAUCAUGAUAUCGGUACCAGUAAA |
| | | GGAGAAGAACUUUUCACUGGAGUUGUCCCAAUUCUUGUUGAAUU |
| | | AGAUGGUGAUGUUAAUGGGCACAAAUUUUCUGUCAGUGGAGAGG |
| | | GUGAAGGUGAUGCAACAUACGGAAAACUUACCCUUAAAUUUAUU |
| | | UGCACUACUGGAAAACUACCUGUUCCAUGGCCAACACUUGUCACU |
| | | ACUUUCGCGUAUGGUCUUCAAUGCUUUGCGAGAUACCCAGAUCAU |
| | | AUGAAACAGCAUGACUUUUUCAAGAGUGCCAUGCCCGAAGGUUAU |
| | | GUACAGGAAAGAACUAUAUUUUUCAAAGAUGACGGGAACUACAA |
| | | GACACGUGCUGAAGUCAAGUUUGAAGGUGAUACCCUUGUUAAUA |
| | | GAAUCGAGUUAAAAGGUAUUGAUUUUAAAGAAGAUGGAAACAUU |
| | | CUUGGACACAAAUUGGAAUACAACUAUAACUCACACAAUGUAUAC |
| | | AUCAUGGCAGACAAACAAAAGAAUGGAAUCAAAGUUAACUUCAA |
| | | AAUUAGACACAACAUUGAAGAUGGAAGCGUUCAACUAGCAGACCA |
| | | UUAUCAACAAAAUACUCCAAUUGGCGAUGGCCCUGUCCUUUUACC |
| | | AGACAACCAUUACCUGUCCACACAAUCUGCCCUUUCGAAAGAUCC |
| | | CAACGAAAAGAGAGACCACAUGGUCCUUCUUGAGUUUGUAACAGC |
| | | UGCUGGGAUUACACAUGGCAUGGAUGAACUAUACAAAGAGAAUU |
| | | CAGAGCUCGGAUCCACUCGAGAUGCAUUAGAACAAAAAUUAUUAU |
| | | CAGAAGAAGAUUUAAAUUAA |
| gfp-mut2-RT- | 23 | [phosphate] CTGCGCGGATCCCGGAAGCGAGGCCAGCTGGCTGCNNNN |
| UMI-AlwNI* | | NNNNCAAATAAATTTAAGGGTAAGTTTTCC |
| Gfp-Fwd-25nt | 24 | TGAATTAGATGGTGATGTTAATGGGC |
| Template-UEI- | 25 | GAGCGGATAACAATTTCACACAGGCACGGGGTGNNACNNGANNCT |
| DraIII-N2x5 | | NNGCNNGATCGGAAGAGCGTCGTGT |
| Template-UEI- | 26 | GAGCGGATAACAATTTCACACAGGCACGGGGTGNNNNACANNNNG |
| DraIII-N4443 | | AGNNNNTCTNNNGATCGGAAGAGCGTCGTGT |
| Template- | 27 | GGAAGACGTAGCAAGGGAGTAGCCAATGTGAATTCACACAGTGTC |
| Calibrator-AlwNI | | TACAAGTACAGGGGCTGGTCGTGACTGGGAAAACCCTGGC |
| Template- | 28 | GGAAGACGTAGCAAGGGAGTAGCCAATGTGAATTCACACAGTGNN |
| Calibrator- | | TCNNTANNCANNAGNNCAGGGGCTGGTCGTGACTGGGAAAACCCT |
| AlwNI-N2x5 | | GGC |
| Template- | 29 | GGAAGACGTAGCAAGGGAGTAGCCAATGTGAATTCACACAGTGNN |
| Calibrator- | | NNTCTNNNNACANNNNGAGNNNCAGGGGCTGGTCGTGACTGGGAA |
| AlwNI-N4443 | | AACCCTGGC |
| Template- | 30 | GGAAGACGTAGCAAGGGAGTAGCCAATGTGAATTCACACAGTGNN |
| Calibrator- | | NNNNNNNNNNNNNCAGGGGCTGGTCGTGACTGGGAAAACCCTGGC |
| AlwNI-N15 | | |
| Tag-NaBAb- | 31 | CACACAGGAAACAGCTATGACCCAGTGTCTGNNNNNNNNTGTGGA |
| AlwNI | | CGCTGTCTCTTATACACATCTGACGCTGCCGACGACGTCGTGACTGG |
| | | GAAAACCC |
| BG-M13-Fwd** | 32 | [BG]-isp18-GGGTTTTCCCAGTCACGACG* |
| Alexa488-M13- | 33 | [Alexa488]-CACACAGGAAACAGCTATGACC |
| Rev | | |

All the oligonucleotides (but 15 obtained by primer extension, 16 and 22) were purchased from Integrated DNA Technologies (IDT). Random sequences corresponding to the UCI or the Calibrator are represented by N arrays and are underlined whereas the UMI is represented by an italicized N arrays. The sequence digested by AlwNI (CAGNNNCTG) and DraIII (CACNNNGTG) are shown in bold.
*These oligonucleotides are 5′ phosphorylated to allow their ligation with another DNA by T4 DNA ligase.
**[BG]: benzyl-guanine; isp18: 18 carbon-long internal spacer.

### Example 1: Cells Preparation, Individualization and Lysis

In this example, we demonstrated that bacterial cells can be individualized in droplets and efficiently lysed using a detergent. E. coli bacteria (T7-Xpress Lys/I^{q}, GFP null strain New England Biolabs) transformed either with a plasmid carrying GFP gene under the control of T7 RNA polymerase promoter or a plasmid bearing an unrelated construct were used as model bacteria and will be later summarized as strains GFP+ and GFP-respectively. Note that both plasmids confer ampicillin resistance to the bacteria allowing for their co-culture.

a. Preparation of the Cell

A cell pre-culture (starter) was first obtained by inoculating a 2YT media supplemented with 0.1 mg/mL ampicillin and 2% glucose, and incubating it over-night at 37° C. and under agitation. 500 μl of this starter (OD600 of 3.77) were then used to inoculate 20 mL of the same medium, and the bacteria were allowed to grow until the culture reached an OD600 of 0.6. Next, the cells were fluorescently stained to allow for monitoring cells during the microfluidic steps. To do so, 2 mL of this culture were stained using 5 μM SYTO 9 (molecular probes by Life technologies, ref 34854) for 2 hours in the dark, following supplier recommendations. Cells were washed once with PBS buffer 1× before being pelleted for further experiments.

b. Cells Encapsulation and Lysis

Experimental Procedure

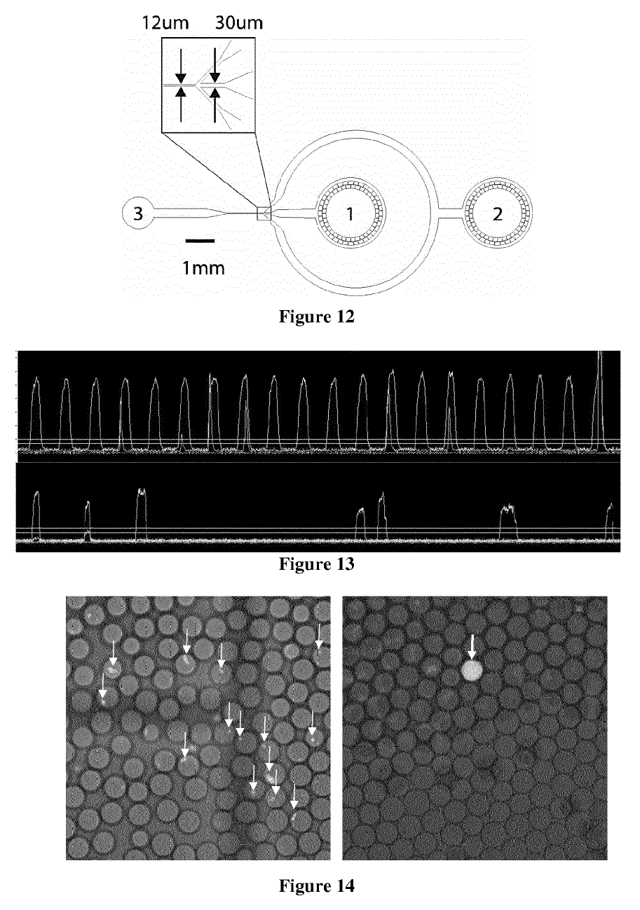

Pellets of labelled cells were diluted to reach an OD600 of 3.75 (giving a droplet occupancy of −20%) in a CutSmart® buffer 1× (50 mM Potassium acetate, 20 mM Tris acetate pH7.9 at 25° C., 10 mM Magnesium acetate and 0.1 mg/mL BSA, New England Biolabs) supplemented with 37.5 μg/mL dextran Texas red 70 000 MW (used as droplet tracker), and 0.05% Pluronic F68. The mixture was then loaded into a 0.5 mL PCR tube containing a magnetic stirring bar (5 mm length and 2 mm diameter) closed by a plug of PDMS. One extremity of the system was connected to a Fluigent infusion device whereas the other extremity was connected to the droplet co-flow generator (FIG. 11, inlet 1). During droplet production, the mixture was stirred by placing a stirring plate (Hei-Mix S, Heidolph instrument) aside the bacteria-containing tube to keep bacteria properly suspended. A solution of CutSmart® buffer 1× supplemented with BPER™ lysis reagent (90% final concentration Thermo Scientific) was loaded into a length of PTFE tubing (I.D. 0.75 mm tubing; Thermo Scientifc) and connected to the Fluigent infusion device at one side of the tubing and to the droplet co-flow generator (FIG. 11, inlet 2) at the other side of the tubing. Both aqueous solutions (bacteria suspension and lysis solution) were combined on-chip prior to being dispersed into a stream of Novec 7500 (3M) fluorinated oil supplemented with 3% Krytox-Jeffamine 1000 diblock copolymer fluorosurfactant (Ryckelynck et al 2015) that was infused into the third inlet (FIG. 11, inlet 3). 4 pL droplets were then produced at a rate of 800 droplets per second by infusing both aqueous phases at 200 nL/min and the oil phase at 1000 nL/min. Droplets were collected at the outlet of the chip (FIG. 11, outlet 4) via a length of tubing into a 0.2 mL PCR tube closed by a plug of PDMS. Moreover, an identical experiment in which the lysis solution was exchanged for a lysis agent-free CutSmart® buffer 1× phase was used for a control experiment. Upon an incubation of 20 min at 25° C., droplet fluorescence was analyzed on-line by re-injecting the droplets into a droplet fluorescence analysis microfluidic device. Droplets contained into a 0.2 mL PCR tube closed by a plug of PDMS were reinjected into a droplet analyzer (FIG. 12, inlet 1) where they were spaced by an oil stream (FIG. 12, inlet 2) and their fluorescence content was analyzed by the optical set-up introduced above. The Texas Red contained into each droplet allows identifying a droplet as an orange peaks (FIG. 13). Moreover, the staining of cell nucleic acids by Syto9 enables both detecting bacteria and appreciating their lysed/integer status. Indeed, the presence of an integer cell is indicated by a spike of green fluorescence into the droplet (orange peak; FIG. 13, top panel), whereas a lysed cell gives a more homogeneous labelling of lower intensity (e.g. second droplet from the left on FIG. 13, bottom panel). To further confirm this result, 10 μL of the droplets were loaded into a plastic Malassez hematimeter and imaged on an epi-fluorescence microscope both in bright-field and green fluorescence (ex/em=470 nm/525 nm) mode (FIG. 14). The green labelling of cell nucleic acids by Syto9 allows discriminating intact bacteria (green rod shapes on FIG. 14, left panel) from lysed cells (homogenous green droplet staining on FIG. 14, right panel). This experiment showed that not only the presence of the lysis agent does not challenge droplets stability, but it also allows to efficiently lyse the cells and release their content into the droplets. Both approaches confirmed that cells can be isolated and lysed in droplets in conditions that do not compromise droplets stability. Moreover, the lysis conditions (i.e. CutSmat® buffer supplemented with B-PER™) are compatible with several molecular biology reactions such as reverse transcription, DNA digestion by restriction enzymes and DNA ligation. This example demonstrates the possibility of lysing cells into the droplets while preserving droplet integrity and being compatible with downstream molecular biology reactions.

### Example 2: Preparation of the Unique Identifier and Droplet Signatures

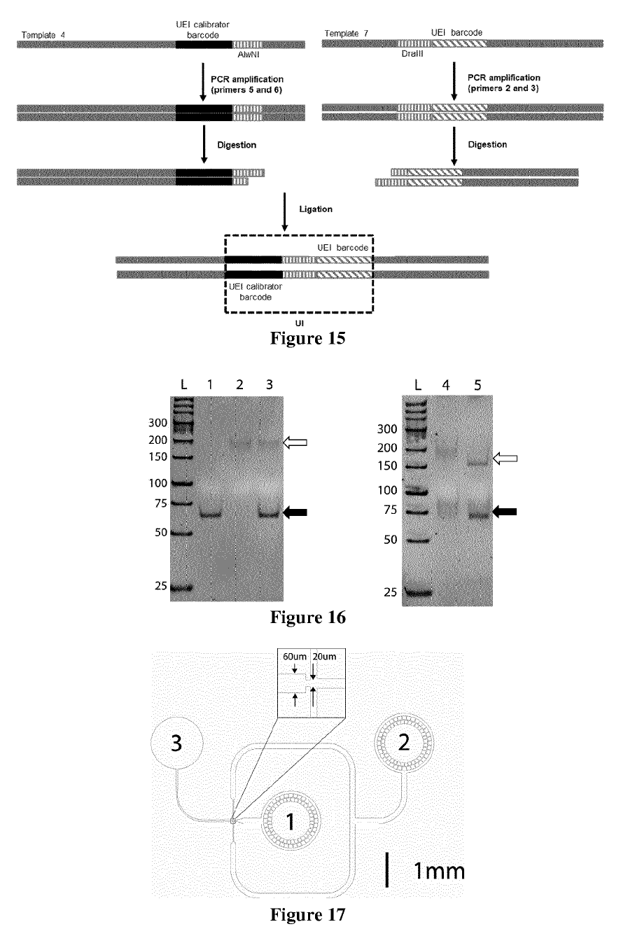

In this example, we show how Unique Identifiers (UIs) can be prepared both in bulk and in microfluidic droplets as well as how they can be used to generate a signature encoding droplet identity. A UI is made of a pair of random sequences (the UEI (“unique entity identifier”) and the UEI Calibrator) surrounded by constant sequence regions encompassing restriction sites generating compatible extremities later used to recombine both sequences together (FIG. 15). To form a UI, both UEI-Calibrator and UEI sequences should be first amplified in a duplex PCR prior to being recombined together through a specific restriction/ligation reaction. Then, the pool of UI contained into the same droplet constitute the signature of the droplet that can later be used to reassign a given encoded molecule to the droplet it originates from.

a. Duplex PCR Co-Amplification of the Two Barcode Sets

We first validated the possibility of performing a duplex PCR co-amplification of UEI-Calibrator and UEI sequences in tubes prior to transferring it in droplet microfluidic format.

Experimental Procedure

500 atto moles of one or both templates (i.e. UEI-DraIII (1) and AlwNI-Calibrator-DraIII (4)) diluted into 0.2 mg/mL yeast total RNA (Ambion) were introduced into a reaction mixture containing 0.2 μM of each forward (molecules 2 and 6) and reverse (molecules 3 and 5) primers, 0.2 mM of each dNTP, 0.1% Pluronic F68, 0.67 mg/mL dextran Texas Red (Invitrogen), 6 nM of a Taq polymerase produced in the laboratory and 1× CutSmart® buffer (50 mM Potassium acetate, 20 mM Tris acetate pH7.9 at 25° C., 10 mM Magnesium acetate and 0.1 mg/mL BSA) in a final volume of 50 μL. The mixture was then thermocycled with an initial denaturation step of 30 sec at 95° C. followed by 25 cycles of 5 sec at 95° C. and 30 sec at 65° C.

Results

Upon amplification, PCR products with the expected size (62 base pairs for UEI-DraIII and 142 base pairs for AlwNI-Calibrator-DraIII) were readily observed on 8% polyacrylamide native gel. Whereas, a single band was observed when only one of the template was present (FIG. 16, lane 1 and 2), two distinguished bands were seen when both templates were mixed together (FIG. 16, lane 3), confirming that both templates can be efficiently co-amplified. In this section only the Calibrators carried a randomized region, which explains the slightly smeary aspect of the band corresponding to the Calibrator. This smear was attributed to the formation of heteroduplexes between the strands of two different Calibrators sharing the same constant regions but having different randomized ones.

b. Amplification of the PCR in Water-in-Oil Droplet

We next verified that the co-amplification can efficiently occur in droplets.

Experimental Procedure

14 atto moles (a concentration allowing for having a theoretical average of 4 template DNA molecules per droplet) of each template (i.e. Template UEI-N15-DraIII (7) and Template AlwNI-N15-Calibrator-DraIII (8)) diluted into 0.2 mg/mL yeast total RNA (Ambion) were introduced in 200 μL of reaction mixture containing 0.2 μM of each forward (molecules 2 and 6) and reverse (molecules 3 and 5) primers, 0.2 mM of each dNTP, 0.1% Pluronic F68, 0.67 mg/mL dextran Texas Red (Invitrogen), 6 nM of a Taq polymerase produced in the laboratory and 1× CutSmart® buffer (50 mM Potassium acetate, 20 mM Tris acetate pH7.9 at 25° C., 10 mM Magnesium acetate and 0.1 mg/mL BSA. The mixture was then loaded into a length of PTFE tubing (I.D. 0.75 mm tubing; Thermo Scientifc) and connected to the Fluigent infusion device at one side of the tubing while the other the other side was connected to a 40 μm deep droplet generator (FIG. 17, inlet 1). An oil phase made of Novec 7500 supplemented with 3% Krytox-Jeffamine 1000 diblock copolymer fluorosurfactant (Ryckelynck et al 2015) was also infused into the chip (FIG. 17, inlet 2) and used to produce 100 pL droplets at a rate of 1600 droplets per second by infusing oil and aqueous phases at 1500 nL/min and 1550 nL/min respectively. Droplets were collected for 11 min via a length of tubing (FIG. 17, outlet 3) into a 0.2 mL PCR tube closed by a plug of PDMS. The tube was then placed in a thermocycler and the emulsion was subjected to an initial denaturation step of 30 sec at 95° C. followed by 25 cycles of 5 sec at 95° C. and 30 sec at 65° C. Upon thermocycling, the emulsion was broken using 50 μL of 1H, 1H, 2H, 2H, perfluoro-1-octanol (Sigma-Aldrich) and 5 μL were loaded on an 8% native PAGE.

Results

Gel analysis confirmed that the duplex PCR amplification properly worked in water-in-oil droplets (FIG. 16, lane 5) and with the same apparent efficiency than in bulk (FIG. 16, lane 4). Moreover, the analysis revealed that compartmentalizing the reaction into droplets allowed obtaining better resolved bands, likely by limiting recombination, the formation of heteroduplexes and other PCR artefacts linked to the presence of the randomized regions on both barcodes (UEI-Calibrators and UEIs).

c. UI-Based Signature

Forming the UI requires recombining together the UEI-Calibrator and the UEI through a restriction/ligation coupled reaction. To do so, we choose two non-palindromic restriction sites producing compatible 3′ overhangs. AlwNI (5′-CAGNNN/CTG-3′) and DraIII (5′-CACNNN/GTG-3′) are two enzymes digesting sequences (bolded in Table 1) fulfilling these criteria. Digestion at these sites generates two compatible sequences that can recombine together and form new sequences (i.e. 5′-CAGNNNGTG-3′ and 5′-CACNNNCTG-3′) that can no longer be digested by the enzymes, pulling therefore the equilibrium toward the formation of recombined molecules.

Experimental Procedure

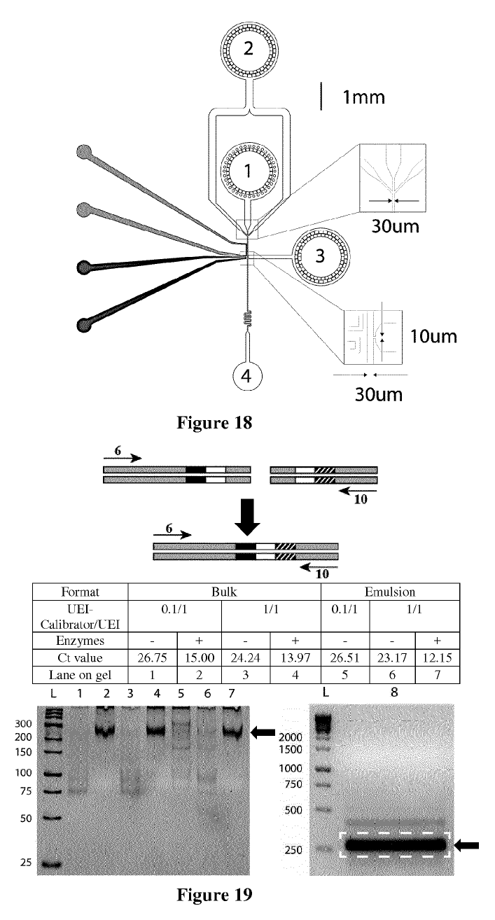

The duplex PCR established in section b was repeated using template molecules 7 and 8 and amplification primers 3, 5 and 6. However, primer 2 was exchanged for primer 9 that allowed introducing Illu-1 sequence, an adaptor sequence later used for next generation sequencing analysis. To evaluate to what extend the relative amount of both barcodes can affect recombination efficiency, so UI formation, we performed a set of experiments using two relative concentrations of UEI-Calibrators and UEI in the droplets. This ratio was changed by varying the concentration of the corresponding primers in the PCR mixture. However, the diversity of the barcodes was preserved by initiating each experiment with the same average number of template per PCR droplet. Therefore, in a first reaction set 14 atto moles of each template diluted into 0.2 mg/mL yeast total RNA (Ambion) were mixed with 0.2 μM of each primer (3, 5, 6 and 10) giving a final ratio UEI-Calibrator/UEI of 1/1. In a second set of reactions, 14 atto moles of each template diluted into 0.2 mg/mL yeast total RNA (Ambion) were mixed with 0.2 μM of primers 3 and 10 and 0.02 μM of primers 5 and 6, giving a final ratio UEI-Calibrator/UEI of 0.1/1. Each template/primers mixture was then introduced into 200 μL of reaction mixture containing 0.2 mM of each dNTP, 0.1% Pluronic F68, 0.67 mg/mL dextran Texas Red (Invitrogen), 6 nM of a Taq polymerase produced in the laboratory and 1× CutSmart® buffer (50 mM Potassium acetate, 20 mM Tris acetate pH7.9 at 25° C., 10 mM Magnesium acetate and 0.1 mg/mL BSA). The mixture was dispersed into 100 pL droplets as above and the emulsion was collected for 20 min in a 0.2 mL tube closed by a plug of PDMS and thermocycled as before. Upon thermocycling, the emulsion was reinjected into a droplet picoinjector (FIG. 18, inlet 1) at 500 nL/min and the droplets were spaced by a stream of Novec 7500 oil supplemented with 2% Krytox-Jeffamine 1000 diblock copolymer fluorosurfactant infused at 1600 nL/min through a second inlet (FIG. 18, inlet 2). A mixture of CutSmart™buffer 1× (50 mM Potassium acetate, 20 mM Tris acetate pH7.9 at 25° C., 10 mM Magnesium acetate and 0.1 mg/mL BSA), supplemented with 1 mM rATP, 7 mM DTT, 3 U/μL DraIII HF (New England Biolabs), 3 U/μL AlwN1 (New England Biolabs), 30 U/μL T4 DNA ligase (New England Biolabs), 20 μM coumarin acetic (used as injection tracker) and 0.1% pluronic F68 was prepared. The mixture was loaded into a length of PTFE tubing (I.D. 0.75 mm tubing; Thermo Scientifc) and connected to the Fluigent infusion device at one side of the tubing and to the third inlet of the chip (FIG. 18, inlet 3) at the other side. Then, 25 pL of the enzyme mixture were delivered to each 100 pL droplet by infusing the “enzyme” phase at 150 nL/min while energizing the electrodes facing the injection point with a squared AC field (400 V, 30 Hz) obtained by a function generator connected to an high voltage amplifier (TREK Model 623B). Droplets were collected (FIG. 18, outlet 4) in a 0.5 mL tube under mineral oil for 45 minutes prior to being incubated overnight at 37° C. to allow digestion and ligation to occur. In parallel, an identical reaction in which enzymes were omitted was performed as a control. Upon incubation, the emulsion was broken with 50 4 of 1H, 1H, 2H, 2H, perfluoro-1-octanol (Sigma-Aldrich) and the labelling efficiency was assessed by quantitative PCR with the kit SsoFast Evagreen Supermix (Bio-Rad) supplemented with primers 6 and 10. The mixture was thermocycled in a CFX qPCR machine (Bio-Rad) with an initial denaturation step of 30 sec at 95° C. followed by 40 cycles of 5 sec at 95° C. and 30 sec at 60° C. At the end of the process, the Ct value was determined for each condition (FIG. 19, Top and middle panels) and the quality of the amplified material was verified by loading an aliquot of each qPCR reaction onto a native polyacrylamide gel (FIG. 19, bottom left panel). After having verified the quality of the DNA, ˜1000 droplets from the emulsion containing the 1/1 UEI-Calibrator/UEI ratio were transferred in a new tube where they were broken with 50 4 of 1H, 1H, 2H, 2H, perfluoro-1-octanol (Sigma-Aldrich) and the released DNA was recovered in 1× CutSmart™ buffer. The DNA was then indexed with primers N503 and N702 using Nextera index kit (ref FC-121-1011, Illumina) following supplier specifications. To limit the risk of unwanted mutations during the indexing step, the reaction was performed using Phusion DNA polymerase (ThermoScientific). The band containing indexed DNA was then purified on a 1% agarose gel electrophoresis and the DNA recovered with the kit Wizard SV Gel and PCR clean up system (Promega) (FIG. 19, right panel). Last, the resulting library was loaded onto an Illumina V2-300 cycles flow-cell and the DNA was sequenced on a MiSeq device (Illumina).

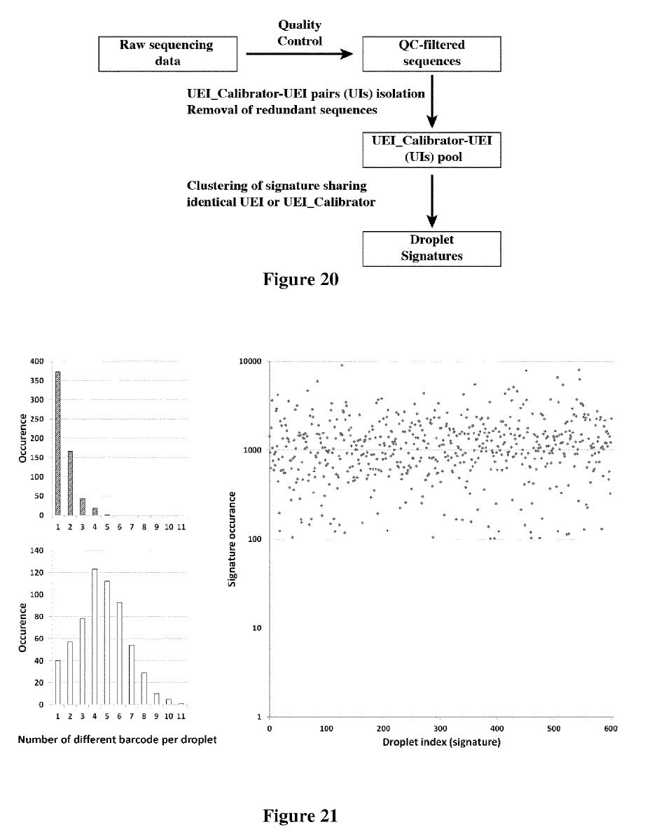

Finally, the sequences were analyzed using a Python-based bioinformatics algorithm (FIG. 20). The algorithm works in 3 main steps. First, raw sequences obtained from the sequencer are quality-filtered and those with a Q score below 30 are removed from the pool. Moreover, sequences presenting mutations in the non-randomized region or showing an inappropriate length are also removed from the pool. Second, UEI-Calibrator and UEI sequences are extracted and pairs coming from the same droplet are clustered together. Briefly, all the UEIs associated with a given UEI-Calibrator are clustered together. Then, all the UEI-calibrator sharing the UEIs contained into the same cluster are also clustered together. At the end of the process, clusters of UIs (pairs of UEI-Calibrators and UEIs) are obtained and form a signature of the droplet. Finally, in a third step, the signatures can be used to reassign each molecule from a pool to the droplet it originates from.

Results

The UI signature readily forms only in the presence of restriction/ligation enzymes. Indeed, the qPCR analysis revealed that whatever the UEI-Calibrator/UEI ratio used, more than 10 additional amplification cycles (delta Ct >10) are required the reach the threshold in the absence of recombination enzymes (FIG. 19, Top panel, compare columns − and + enzymes), indicating that there is at least a thousand times less recombined material in the absence of enzymes. Moreover, electrophoresis gel analysis (FIG. 19, left panel) showed that specific band of the expected size is obtained only in the presence of the enzymes (lanes 2, 4 and 7). Therefore, UI formation is highly controlled as it occurs only in the presence of specific enzymes and does not form spontaneously form during the PCR reaction, which limits the risk of forming UI in non-specific way. Not only this recombination was found to work in tubes (FIG. 19, left panel, lanes 1 to 4) but it also works in droplets (FIG. 19, left panel, lanes 5 to 7) starting from PCR amplification products also prepared in droplets. As in tubes, the presence of UI in droplets requires the presence of the specific restriction/ligation enzymes (compare lanes 6 and 7).

Further analyzing the droplet-contained UI by Next Generation Sequencing together with the bioinformatics algorithm shown on FIG. 20, allowed us to reassign each UEI and each UEI-Calibrator to the droplet it originates from (FIG. 21). Indeed, the use of a barcode made of 15 randomized (theoretical diversity of 10⁹ different variants) region makes unlikely to find the same UEI or the same UEI-Calibrator in more than one droplet in the emulsion (made at most of a few millions of droplets), making this reassignment faithful. The number of different UEI and UEI-Calibrator per droplet followed a Poisson distribution (FIG. 21, left; see also FIG. 23) as expected for objects randomly distributed into compartments such as droplets. This confirms that the proper confinement of the information was preserved all along the process and that the UI were generated in the droplets and not after droplet breaking. Note that in this example, the average number of different UEI-Calibrator per droplet was only 1 while 4 were expected. However, complementary experiments presented hereafter (section d) allowed readjusting this parameter and further confirming the Poisson-driven behavior of the molecules.

Clustering the different UIs contained in each droplet allows obtaining a signature unique to each droplet. Interestingly, the signatures were evenly represented within the pool of sequences (FIG. 21, right), indicating that no significant droplet-to-droplet bias occurs during UI preparation.

d. Adjusting UEI Diversity

To further confirm that both UEI-Calibrators and UEIs distributes into the droplets following a Poisson distribution, as well as to properly adjust the content of each type of barcodes, the experiment was performed at three different average number (lambda values) of barcode per droplet.

Experimental Procedure

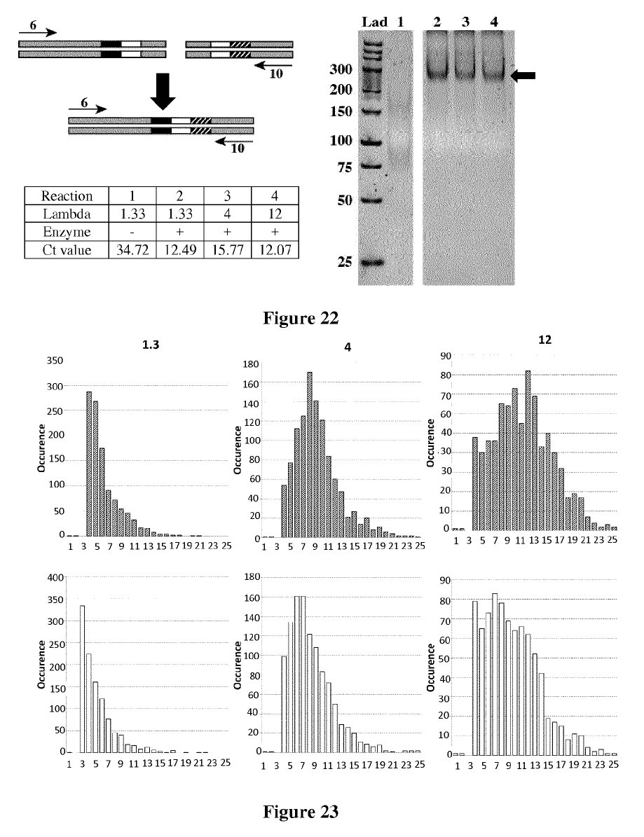

As before, a duplex PCR was performed but using different starting amount of template molecules 7 and 8. To test the lambda 1.3 condition (i.e. an average of 1.3 UEI-Calibrator and 1.3 UEI per droplet), 5 atto moles of each template (i.e. Template UEI-N15-DraIII (7) and Template AlwNI-N15-Calibrator-DraIII (8)) diluted into 0.2 mg/mL yeast total RNA (Ambion) were introduced into 200 μL of a reaction mixture containing 0.2 μM of each forward (molecules 2 and 6) and reverse (molecules 3 and 5) primers, 0.2 mM of each dNTP, 0.1% Pluronic F68, 0.67 mg/mL dextran Texas Red (Invitrogen), 6 nM of a Taq polymerase produced in the laboratory and 1× CutSmart® buffer (50 mM Potassium acetate, 20 mM Tris acetate pH7.9 at 25° C., 10 mM Magnesium acetate and 0.1 mg/mL BSA. To test the lambda 4 (i.e. an average of 4 different UEI-Calibrators and 4 different UEIs per droplet) and lambda 12 (i.e. an average of 12 different UEI-Calibrators and 12 different UEIs per droplet) conditions, the starting amount of each template introduced in the reaction mixture was respectively raised to 14 atto moles and 56 atto moles. 100 pL droplets were generated at a frequency of 300 droplets per second using the same chip as before (FIG. 17) and the emulsion was collected and thermocycled as in section c. Finally, as above, an enzyme mixture was picoinjected into each droplet and the emulsion was incubated to allow the recombination to occur. Moreover, a control reaction in which restriction/ligation enzymes were omitted was performed in parallel with the lambda 1.3 condition. Upon incubation, the proper recombination was again verified by qPCR (using primers 6 and 10) and gel electrophoresis (FIG. 22). Moreover, for each condition, ˜1000 droplets were transferred into a new tube where they were broken by adding 20 μL of 1H, 1H, 2H, 2H, perfluoro-1-octanol (Sigma-Aldrich) and the released DNA was recovered in 200 μL of PCR mixture containing Q5 DNA polymerase (New England Biolabs) and its buffer at the recommended concentration, 0.2 mM of each dNTP and a Nextera primer pair (N703 and N502 to index the lambda 1·3 condition; N705 and N504 to index the lambda 4 condition; N706 and N517 to index the lambda 12 condition; Illumina) at the recommended concentration. Indexing was performed by thermocycling the mixture using the program recommended by the manufacturer and the amplification product was loaded on a 1% agarose gel). Upon electrophoresis, the band containing the indexed DNA was sliced from the gel and the DNA was recovered with the kit Wizard SV Gel and PCR clean up system (Promega). Finally, the resulting library was loaded into an Illumina V2-300 cycles flow-cell and a high-throughput sequencing was performed on a MiSeq device (Illumina). Then, sequencing data were analyzed using our bioinformatics algorithm (FIG. 20) and the distribution profiles of both UEI-Calibrators and UEIs were established for each condition (FIG. 23).

Results

Preparing UIs in droplets while varying the initial number of UEI-Calibrator and UEI template per droplet (lambda value) did not significantly challenged the process per se. Indeed, whatever the starting lambda value (from 1.3 to 12), very close Ct values were obtained when quantifying the amount of DNA amplified and recombined into the droplets at the of the process (reactions 2 to 3 on FIG. 22). With respect to the enzyme-free control (reaction 1 on FIG. 22), up to 20 less amplification cycles were required to reach the threshold when enzymes were added, indicating that there was up to a million times more UI formed in the presence of enzymes. Analysis on gel, confirmed that the proper reaction product (indicated by an arrow on FIG. 22) was observed only in the presence of the enzyme (lanes 2 to 4), whereas only a small parasitic amplification product was obtained in the absence of enzymes (lane 1).

Analyzing the sequence content of an aliquot of 1000 droplets from each condition with our bioinformatics algorithm revealed that, as expected, changing the starting amount of UEI-Calibrator and UEI template per droplet directly impacts the distribution of both barcodes in the droplets (FIG. 23) and that the higher the starting number of different UEI-Calibrator and UEI per droplet the higher the number of different UI forming droplet signature. Therefore, adjusting the initial concentration of both templates allows adjusting the diversity of UI per droplet (so the complexity of the signature) in a predictable way.

### Example 3: Labelling and Quantifying DNA Using UI/Signature

Since UIs are obtained through a digestion/ligation process, they can also be appended to another DNA molecule provided it possesses a site compatible with one of the restriction products. This DNA could be either a reverse transcription product or a DNA attached to another molecule such as a protein. In this example, we demonstrate the possibility of attaching UI to another DNA and to later use our approach to reassign each DNA molecule to the droplet it originates from as well as quantifying the DNA content of each droplet. Therefore, in this example we aimed to: i) isolate and PCR co-amplify UEI-Calibrator and UEI templates; ii) supplement each UEI-Calibrator/UEI-containing droplet with a DNA displaying an extremity compatible with one of the restriction products; iii) add restriction/ligation enzymes to each droplet to form UIs through recombination and attach them at the extremity of the target DNA molecule; iv) index and sequence the library in high throughput regimes. Furthermore, a random sequence of 8 nucleotides forming a Unique Molecular Identifier (UMI) was inserted into the target DNA to allow the later counting of the molecules contained in each droplet.

a. UEI Formation and DNA Labeling in Tubes

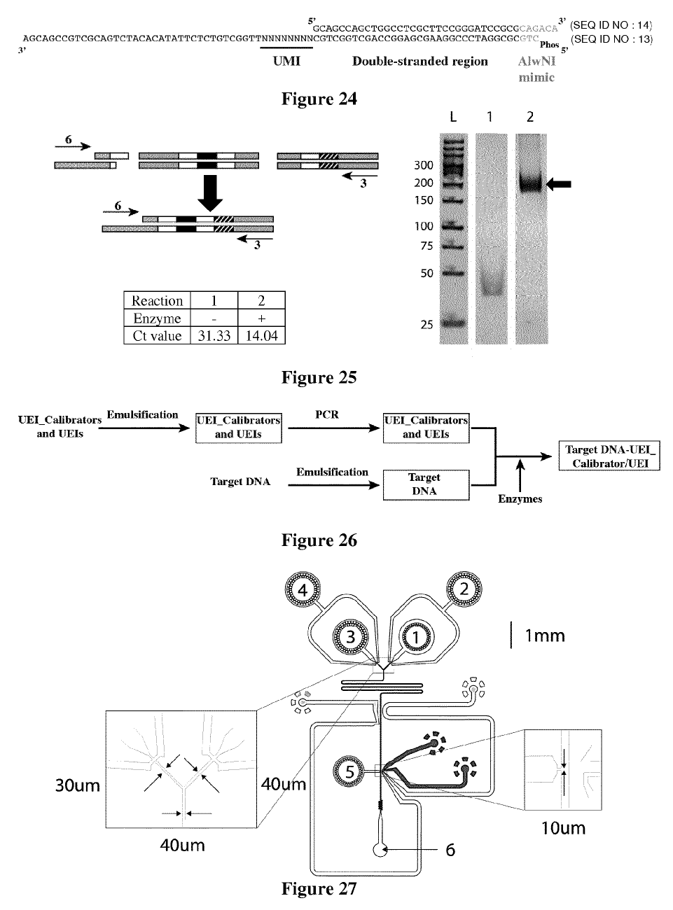

Prior to performing DNA labelling in droplets, we first verified the whole reaction process in tubes. In this example, we used a partially double stranded DNA mimicking a reverse transcription product (FIG. 24). This molecule is made of a long molecule (13) whom the 3′ part is kept single stranded and bears a randomized region of 8 nucleotides (UMI) at its 5′ part followed by a constant region forming a double strand by pairing with the molecule 14. This pairing generates a 3′ overhang compatible with the DraIII cleavage product of the UEI-Calibrator moiety of the UIs. Moreover, the 5′ phosphorylated end of 13 makes it substrate of DNA ligases.

Experimental Procedure

UI components (UEI-Calibrators and UEIs) were first generated as in Example 2 by a duplex PCR co-amplification. To do so, 500 atto moles of template DNAs 7 and 11 were first PCR amplified in a volume of 200 μL as described in Example 2 section a using 0.2 μM of primers 2, 3, 5 and 12. Target DNA was prepared by annealing together 1.25 μmol of the molecule 13 and 1.25 μmol of the molecule 14 in 20 μL of 1× CutSmart® buffer, 0.25 mM dNTP, 10 mM DTT, 0.1 μM FAM, 0.15 mg/mL dextran Texas Red. The enzyme mixture was prepared by supplementing a 1× CutSmart® buffer with 6 mM rATP, 60 mM DTT, 8 U/μL DraIII HF, 8 U/μL AlwN1, 80 U/μL T4 DNA ligase, 55 μM coumarin acetic and 0.3% Pluronic F68. 10 μl of duplex PCR were then mixed with 4 μL of target DNA solution and 3 μL of Enzyme mixture and the resulting mixture was incubated overnight at 37° C. As a control, the same reaction was performed but restriction enzymes and the ligase were omitted from the enzyme mixture. Upon incubation, an aliquot of each reaction was analyzed by quantitative PCR using the kit Sso-Fast Evagreen (Bio-Rad) supplemented in primers 3 and 6. After the qPCR, an aliquot of each reaction was analyzed on polyacrylamide gel electrophoresis.

Results

The annealing of molecule 13 and 14 form a target DNA displaying a 3′ overhang compatible with the DraIII product generated upon UEI-Calibrator digestion. Mixing together this target DNA with co-amplified UEI-Calibrators and UEIs as well as with restriction and ligation enzymes allows the formation of UIs and their attachment to the target DNA, therefore directly encoding it. qPCR analysis indicates that more than 17 additional amplification cycles (delta Ct >17) are required the reach the threshold in the absence of recombination enzymes (FIG. 25, compare columns − and + enzymes), indicating that there is at least a 160,000-times less UI-encoded DNA molecules in the absence of enzymes. Moreover, gel electrophoresis analysis (FIG. 25, right panel) showed that specific band of the expected size is obtained only in the presence of the enzymes (lane 2).

Therefore, UIs can be used to encode a DNA displaying an overhang compatible with one of the restriction site present in the UI.

b. UI Formation and DNA Labeling in Droplet

We next transposed the DNA labelling into the droplet microfluidic format. As in Example 2, UEI-Calibrators and UEIs were co-amplified in droplets using a duplex PCR prior to adding the DNA to label and the enzyme mixture to each droplet.

Experimental Procedure

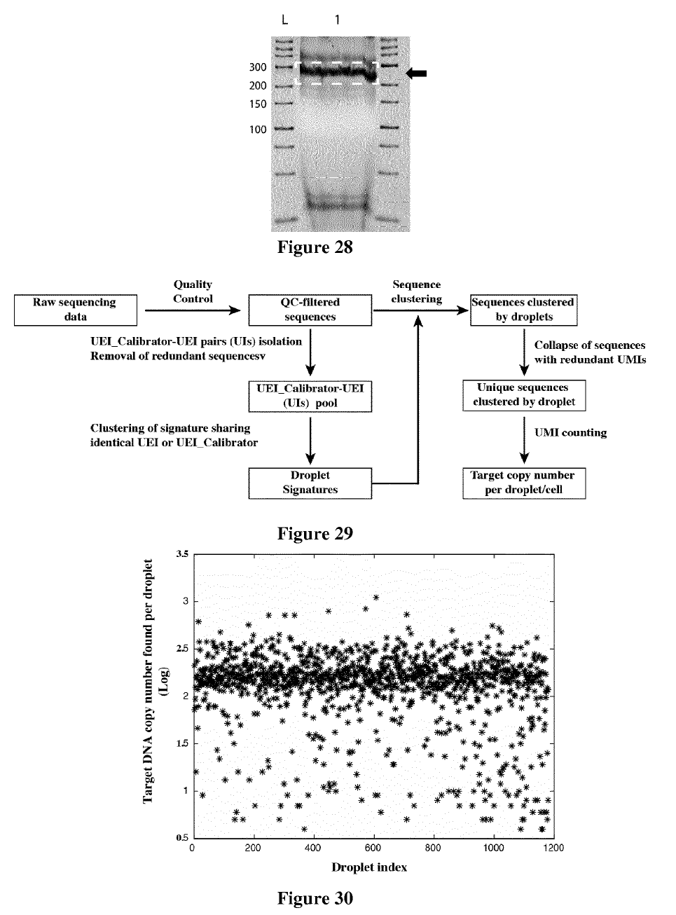

An extended version of the molecule 7 encompassing the sequencing adapter Illu1 was first prepared by primer extension. Briefly, 10 μmols of the template 7 were mixed with 50 μmols of primer 9 in a 50 μL reaction mixture containing 0.2 mM of each dNTP, 1U of Phusion DNA polymerase (ThermoScientific) and the corresponding buffer at the recommended working concentration. The mixture was then incubated for 5 minutes at 98° C. and 10 minutes at 72° C. Extension product was then purified on an agarose gel and the DNA recovered with the Wizard SV Gel and PCR clean up system (Promega). The extended molecule is now called Template UEI-N15-DraIII-Illu1 and corresponds to molecule 15. 14 atto moles (a concentration allowing for having an average of 4 template DNA molecules per droplet) of each template (i.e. Template UEI-N15-DraIII-Illu1 (15) and Template-AlwNI-N15-Calib-DraIII (11)) diluted into 0.2 mg/mL yeast total RNA (Ambion) were introduced into a reaction mixture containing 0.2 μM of each forward (molecules 2 and 5) and reverse (molecules 10 and 12) primers, 0.2 mM of each dNTP, 0.1% Pluronic F68, 0.67 mg/mL dextran Texas Red (Invitrogen), 6 nM of a Taq polymerase produced in the laboratory and 1× CutSmart® buffer (50 mM Potassium acetate, 20 mM Tris acetate pH7.9 at 25° C., 10 mM Magnesium acetate and 0.1 mg/mL BSA) in a final volume of 200 μL. The mixture was then loaded into a length of PTFE tubing (I.D. 0.75 mm tubing; Thermo Scientifc) and connected to the Fluigent infusion device at one side of the tubing while the other the other side was connected to a 40 μm deep droplet generator (FIG. 17, inlet 1). An oil phase made of Novec 7500 supplemented with 3% Krytox-Jeffamine 1000 diblock copolymer fluorosurfactant (Ryckelynck et al 2015) was also infused into the chip (FIG. 17, inlet 2) and used to produce 100 pL droplets at a rate of 1600 droplets per second by infusing oil and aqueous phases at 1500 nL/min and 1550 nL/min respectively. Droplets were collected (FIG. 17, outlet 3) for 11 min via a length of tubing into a 0.2 mL PCR tube closed by a plug of PDMS. The tube was then closed, placed into a thermocycler and the emulsion was subjected to an initial denaturation step of 30 sec at 95° C. followed by 25 cycles of 5 sec at 95° C. and 30 sec at 65° C. Target DNA was prepared by mixing and annealing together 33 femto moles of oligonucleotide 13 (corresponding to 1000 DNA molecule per 20 pL) and 66 femto moles of oligonucleotide 14 in 400 μL of solution containing a 1× CutSmart® buffer, 0.25 mM dNTP, 10 mM DTT, 0.1 μM FAM (droplet tracker fluorescent dye), 0.15 mg/mL dextran Texas red, 0.005% Pluronic F68. The mixture was then loaded into a length of PTFE tubing (I.D. 0.75 mm tubing; Thermo Scientifc) and connected to the Fluigent infusion device at one side of the tubing while the other the other side was connected to a 15 μm deep droplet generator (FIG. 17, inlet 1). An oil phase made of Novec 7500 supplemented with 3% Krytox-Jeffamine 1000 diblock copolymer fluorosurfactant was also infused into the chip (FIG. 17, inlet 2) and used to produce 20 pL droplets at a rate of 1600 droplets per second by infusing oil and aqueous phases at 1200 nL/min and 900 nL/min respectively. Droplets were collected (FIG. 17, outlet 3) into a 0.2 mL PCR tube closed by a plug of PDMS for via a length of tubing. The enzyme mixture was prepared by mixing together 9 mM rATP, 30 mM DTT, 3 U/μL DraIII HF (New England Biolabs), 3 U/μL AlwN1 (New England Biolabs), 15 U/μL T4 DNA ligase (New England Biolabs), 20 μM coumarin acetic (injection tracker) and 0.1% pluronic F68 in a 1× CutSmart® buffer. The enzyme mixture was loaded into a length of PTFE tubing (I.D. 0.75 mm tubing; Thermo Scientifc) and connected to the chip (FIG. 27, inlet 5). Both emulsions (UEI-Calibrator/UEI-containing 100 pL droplets and target DNA-containing 20 pL droplets) were combined, were fused and supplemented with the enzyme mixture on a Barcoding microfluidic device (FIG. 27). UEI-Calibrator/UEI-containing 100 pL droplets were reinjected (FIG. 27, inlet 1) at a frequency of ˜100 droplets per second by infusing the emulsion at 400 nL/min while spacing them with a stream of oil (Novec 7500 supplemented with 2% Krytox-Jeffamine 1000 diblock copolymer fluorosurfactant) infused into the chip (FIG. 27, inlet 2) at a flow-rate of 1300 nL/min. Target DNA-containing 20 pL droplets were reinjected (FIG. 27, inlet 3) at a frequency of ˜100 droplets per second by infusing the emulsion at 100 nL/min while spacing them with a stream of oil (Novec 7500 supplemented with 2% Krytox-Jeffamine 1000 diblock copolymer fluorosurfactant) infused into the chip (FIG. 27, inlet 4) at a flow-rate of 1200 nL/min. Pairs of droplets were allowed to form while the droplets were circulating into a short delay line. Pair-wised droplets were then fused when passing in front of an electrode pair to which a squared AC field (800 V, 30 Hz) was applied using a function generator connected to a high voltage amplifier (TREK Model 623B). At the same time, 25 pL of enzyme mixture was delivered to each droplet by injecting the enzyme mixture into the chip (FIG. 27, inlet 5) and infusing it at 100 nL/min. Fused and picoinjected droplets were collected (FIG. 27, outlet 6) for 20 minutes in a 0.5 mL tube under mineral oil. The emulsion was then incubated in a thermocycler and subjected to 15 repeats of the program: incubate for 15 min at 37° C. and 45 min at 16° C. Upon incubation, 1000 droplets were transferred in a new tube where they were broken by adding 20 μL of 1H, 1H, 2H, 2H, perfluoro-1-octanol (Sigma-Aldrich) and the released DNA was recovered in 200 μL of PCR mixture containing Q5 DNA polymerase (New England Biolabs) and its buffer at the recommended concentration, 0.2 mM of each dNTP and Nextera primers N701 and N502 (Illumina) at the recommended concentration. Indexing was performed by thermocycling the mixture using the program recommended by the manufacturer and the amplification product was loaded on a 1% agarose gel (FIG. 28). Upon electrophoresis, the band containing the indexed DNA was then recovered and the DNA isolated with the kit Wizard SV Gel and PCR clean up system (Promega). Finally, the resulting library was loaded onto an Illumina V2-300 cycles flow-cell and the DNA was sequenced on a MiSeq device (Illumina). Upon sequencing, data were analyzed using an up-graded version of our bioinformatics algorithm (FIG. 29). Indeed, first, droplet signatures were established as previously (see Example 2-section c and FIG. 20). Next, these signatures were used to cluster the QC-filtered sequences and reassign each cluster to the droplet it originates from. Then, all the sequences sharing the same UMI sequence (stretch of 8 randomized nucleotides carried by the target DNA) were collapsed into a single sequence. Finally, counting the number of different UMIs contained into each droplet gave access to the absolute quantification of the DNA content of each droplet (FIG. 30).

Results

In this example, we prepared an emulsion made of 20 pL droplets, each containing on average ˜1000 target DNA molecules. In parallel, UEI-Calibrator/UEI-containing droplets were prepared as described in Example 2. Finally, using our Barcoding microfluidic device (FIG. 27) each target DNA-containing droplet was fused to a single UEI-Calibrator/UEI-containing and received a controlled amount of restriction/ligation enzyme mixture. Upon sequencing analysis of the content of ˜1000 droplets we applied an up-graded version of our bioinformatics algorithm (FIG. 29) and succeed in: i) identifying the different UIs contained in the sample and cluster them into droplet signatures and ii) use these signatures to reassign each sequence to the droplet it originates from and finally iii) count the target DNA copy number present into each droplet (FIG. 30). The analysis led us to identify ˜1200 different droplets, a value approaching closely the 1000 expected droplets. Moreover, an average of 200 copies of the target DNA were found in each droplet, again a value closing approaching the theoretical droplet content (expected to be ˜1000 copies per droplet). DNA copy number distribution was very tight, indicating a high homogeneity of the barcoding process (i.e. formation and addition of UIs) among the different droplets. Each sequence-UMI-UI was found at least 10 times in the sequence pool, indicating that the library was analyzed with a 10-times coverage. This indicates that the tight distribution of the droplets content does results from the saturation of the methods and is rather representative of the actual content of the emulsion. Taken together, the results presented in this example show that our methodological and analytical algorithm allows for quantifying the DNA content of a droplet in reproducible way by attaching an UI to it.

### Example 4: Converting RNA into cDNA Encodable by an UI

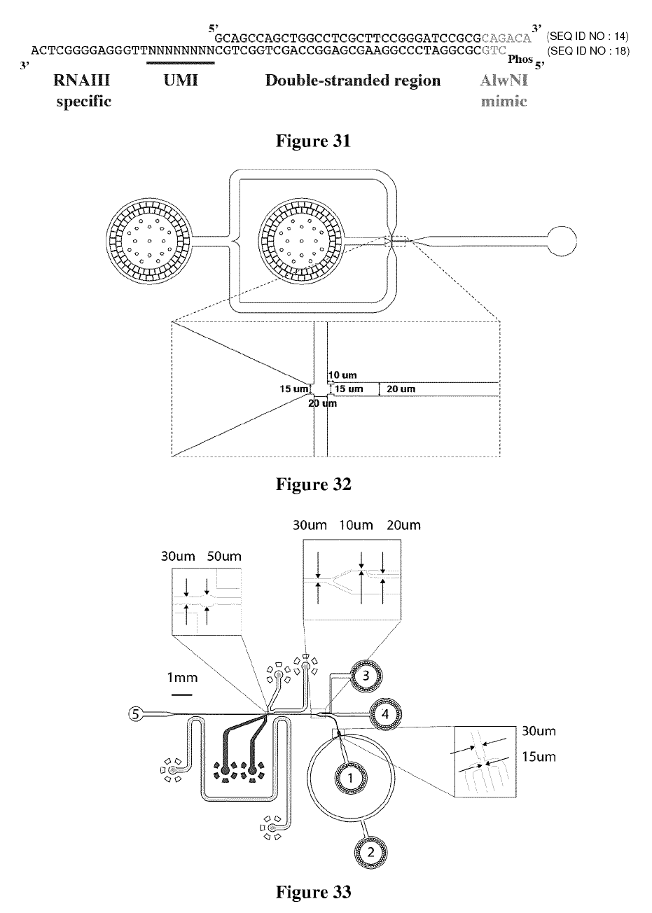

Since our methodology allows for labelling and counting the DNA molecule contained into the droplets of an emulsion, we next investigated if RNA can be converted, in the droplets, into a cDNA that could be used as a substrate of our labelling technology. To generate such cDNA, we designed composite reverse transcription (RT) primers made of i) double-stranded 5′ part terminated by a 3′ overhang compatible with the restriction site present at the extremity of the UI; followed by ii) a single-stranded region containing 8 random nucleotides and working as Unique Molecular Identifier (UMI) and iii) terminated by 3′ single-stranded region annealing specifically to the target RNA (FIG. 31). In this example, we show that such molecule can be used to prime reverse transcription of an RNA as well as attachment point for an UI.

a. Reverse Transcription of RNAs in Droplets

To test the possibility of reverse transcribing RNAs in the droplets, we choose the highly structured (therefore challenging to reverse transcribe) RNA III from Staphylococcus aureus (Benito et al, 2000). The RNA was prepared by in vitro transcription using T7 RNA polymerase using the same procedure described in (Ryckelynck et al, 2015). Upon transcription, RNA was purified on a size exclusion column Nap5 (GE-Healthcare) and quantified using a Nanodrop device. To prevent unwanted reverse transcription of the RNA prior to its encapsulation in droplets, RNA and RT reagents were first emulsified separately and mixed together through droplet fusion (Mazutis et al, 2009). Moreover, to evaluate the sensitivity of the reverse transcription process, we prepared emulsions containing 10, 100 or 1000 RNA molecules per droplet. Then the reverse transcription was added to each droplet and the RT allowed to proceed prior to analyzing the produced cDNA.

Experimental Procedure

0.6, 6 or 60 femto moles of RNA-III (allowing for having respectively, 10, 100 or 1000 RNA molecule per 2 pL droplet) were introduced into a 100 μL of a solution containing CutSmart® buffer, 1.5 mg/mL Dextran Texas Red (Invitrogen), 0.25% Pluronic F68. The mixture was then loaded into a length of PTFE tubing (I.D. 0.75 mm tubing; Thermo Scientifc) and connected to the Fluigent infusion device at one side of the tubing while the other the other side was connected to a 10 μm deep droplet generator (FIG. 32, inlet 1). An oil phase made of Novec 7500 supplemented with 3% Krytox-Jeffamine 1000 diblock copolymer fluorosurfactant was also infused into the chip (FIG. 32, inlet 2) and used to produce 2 pL droplets at a rate of 9000 droplets per second by infusing oil and aqueous phases at 1450 nL/min and 700 nL/min respectively. Droplets were collected (FIG. 32, outlet 3) via a length of tubing into a 0.2 mL PCR tube closed by a plug of PDMS. 200 μL of reverse transcription mixture were prepared by supplementing a CutSmart® buffer prepared at the recommended concentration with 1.25 μmols of RT-UMI-RNAIII primer (molecule 18), 2.5 μmol of Anti-SBACA primer (molecule 14), 0.25 mM of each dNTP, 10 mM DTT, 0.1 μM FAM (droplet tracker) and 125 U of Reverse Transcriptase Maxima RNase H minus (Thermo Scientific). The mixture was loaded into a length of PTFE tubing (I.D. 0.75 mm tubing; Thermo Scientifc) and connected to the Fluigent infusion device at one side of the tubing while the other the other side was connected to a 15 μm deep droplet fusion device (FIG. 33, inlet 1). An oil phase made of Novec 7500 supplemented with 2% Krytox-Jeffamine 1000 diblock copolymer fluorosurfactant was also infused into the chip (FIG. 33, inlet 2) and used to produce 18 pL droplets at a frequency of 1200 droplets per second by infusing oil and aqueous phases at 1280 nL/min and 800 nL/min respectively. Moreover, the 2 pL droplets containing the RNA were also reinjected into the same chip (FIG. 33, inlet 3) at a frequency of ˜1200 droplets per second by infusing them at 140 nL/min and spacing them with a stream of surfactant-free Novec 7500 fluorinated oil (3M) infused into the chip (FIG. 33, inlet 4) at 1400 nL/min. Pairs of droplet were formed and fused by a squared AC field (450 V, 30 Hz) that was applied to built-in electrodes and obtained from a function generator connected to a high voltage amplifier (TREK Model 623B). Fused droplets were then collected (FIG. 33, outlet 5) under mineral oil. In parallel, the same reaction was performed in bulk by mixing 1 volume of RNA dilution with 9 volumes of reverse transcription mixture and by incubating both the bulk mixture and the emulsions for 1 hour at 55° C. Furthermore, a second control reaction in which the reverse transcriptase was omitted was also performed. Upon incubation, emulsions were broken using of 1H, 1H, 2H, 2H, perfluoro-1-octanol (Sigma-Aldrich) and the aqueous phases were recovered. cDNA obtained from the different conditions (bulk or emulsion; 10, 100 or 1000 RNAs per droplet) was then analyzed by qPCR using the SsoFast Evagreen Supermix kit (Bio-Rad) supplemented with the primers 20 and 21. Moreover, the amplification products were analyzed on an 8% native polyacrylamide gel.

Results

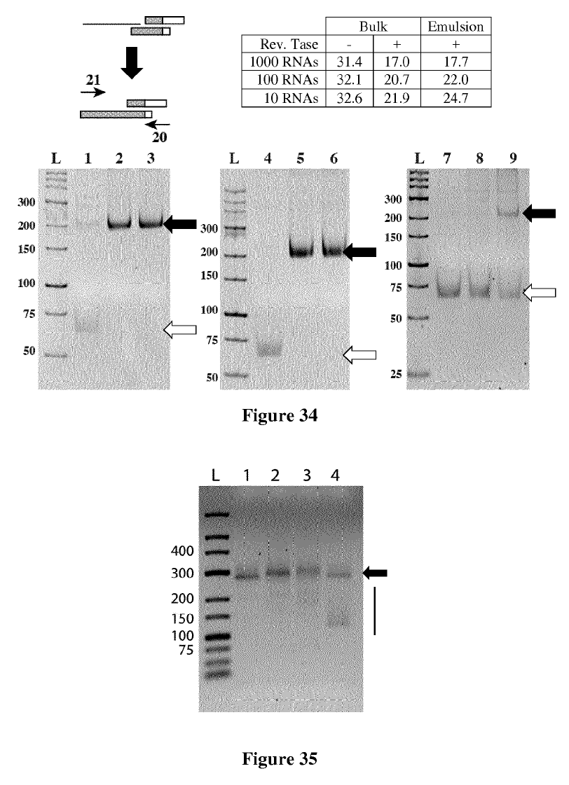

In this example, emulsions of 2 pL droplets containing each 10, 100 or 1000 molecules of RNA-III were produced and fused to 18 pL droplets containing an RT mixture. As a control, the same experiment was performed in a bulk format. Upon incubation, qPCR analysis revealed that RT occurred with the same efficiency both in bulk and emulsified format (FIG. 34). Indeed, in both formats Ct values obtained with bulk and emulsified reactions were very close and significantly higher than that of a control reaction where the reverse transcriptase was omitted. Moreover, the analysis on gel confirmed that the qPCR product obtained in both conditions had the expected size, whereas only a low size PCR side product was obtained with the negative control. Interestingly, in the condition where the RNA was the most diluted (10 molecules per 2 pL) gave a detectable signal corresponding to an amplification product of the expected size only in emulsion demonstrating the higher sensitivity offered by the droplet format and suggesting that if a target RNA is present in at least 10, it should be possible to convert it into a cDNA displaying an extremity compatible with the grafting of a UI. Indeed, in this example we validated the use of an RT primer possessing the sequences elements allowing for efficiently grafting an UI using the strategy validated in Example 3 (compare FIGS. 24 and 31).

### Example 5: Exploring Alternative Format of UEI-Calibrator and UEI Sequences

We noticed that, when working at low concentration of target molecules, some non-specific recombination events occur between some primers and the randomized regions of the UEI-Calibrators and the UEIs. We therefore explored alternative sequences to the simple stretch of 15 contiguous randomized nucleotides. In this example, we reverse transcribed a purified gfp mRNA prior to subjecting the obtained cDNA the labeling by UI produced from templates: i) deprived of randomized regions; ii) or bearing a stretch of 15 contiguous randomized nucleotides (N15); iii) or bearing a stretch of semi-randomized nucleotides where 5 randomized dinucleotides are spaced by constant dinucleotides (N2×5); iv) or bearing a stretch of semi-randomized nucleotides where 4 randomized trinucleotides are spaced by constant trinucleotides (N4443).

Reverse Transcription of the GFP RNA and UI Addition

Experimental Procedure

gfp mRNA was prepared by in vitro transcription using T7 RNA polymerase using the same procedure described in (Ryckelynck et al, 2015). Upon transcription, RNA was purified on a size exclusion column Nap5 (GE-Healthcare) and quantified using a Nanodrop device. 16.67 femto moles of purified gfp mRNA were reverse transcribed in 20 μL CutSmart® buffer prepared at the recommended concentration and supplemented with 1.25 μmol of RT primer (molecule 23), 1.25 μmol of the complementary oligonucleotide (molecule 24), 1 mM dNTP, 10 mM DTT, 10 U of AMV reverse transcriptase, 0.1 μM FAM. The mixture was then incubated 1 hour at 42° C. 500 atto moles of pairs of template UEI-Calibrators and UEIs (i.e. 1/27, 25/28, 26/29 and 7/30) were co-amplified in 200 μL of amplification mixture supplemented with 0.2 μM of each primer (2, 5, 10 and 12) as described in Example 3-section a. Upon thermocycling, 10 μL duplex PCR were mixed with 4 μL of reverse transcribed reaction and 3 μL of Enzyme mixture (lx CutSmart® buffer with 6 mM rATP, 60 mM DTT, 8 U/μL DraIII HF, 8 U/μL AlwN1, 80 U/μL T4 DNA ligase, 55 μM coumarin acetic and 0.3% pluronic F68). The mixture was then subjected to 5 cycles of temperature: 15 min at 37° C. and 45 min at 16° C. At the end of the incubation, 1 μL of each reaction was amplified by PCR using the kit Sso-Fast Evagreen (Bio-Rad) supplemented in primers 3 and 6 and the amplification products were analyzed on a 1% agarose gel (FIG. 35).

Results & Conclusions

The target RNA (gfp mRNA) was properly reversed transcribed and the 4 types of barcodes (UEI-Calibrator/UEI) were appended to the resulting cDNA. Whereas barcodes deprived of randomized regions or affording N2×5 semi-randomized regions gave homogeneous PCR products (lanes 1 and 2 on FIG. 35), more smeary bands were observed with the N4443 and N15 regions. Moreover, secondary amplification products of smaller size tend to significantly accumulate with N4443 and N15. Therefore, semi-randomized barcodes such as the N2×5 represent an attractive alternative to the more conventional N15 barcode design.

### Example 6: Labelling Proteins with UIs

To extend the use of the technology to molecules other than nucleic acids, we validated in this example the capacity of our method to encode a DNA fragment covalently attached to a protein. To this end, we cloned a fusion gene made of protein A and SNAP tag (New England Biolabs) coding regions. The resulting gene was overexpressed in E. coli and the resulting protein (NaBAb) was purified. In this example, we first verified that a unique fragment of DNA could be covalently attached to the protein and that it did not interfere with the capacity of the protein to interact with antibodies via its protein A moiety. Then, we tested if a UI could be attached to the DNA strand appended to the protein.

a. Labeling Antibodies with a Single DNA Per Antibody

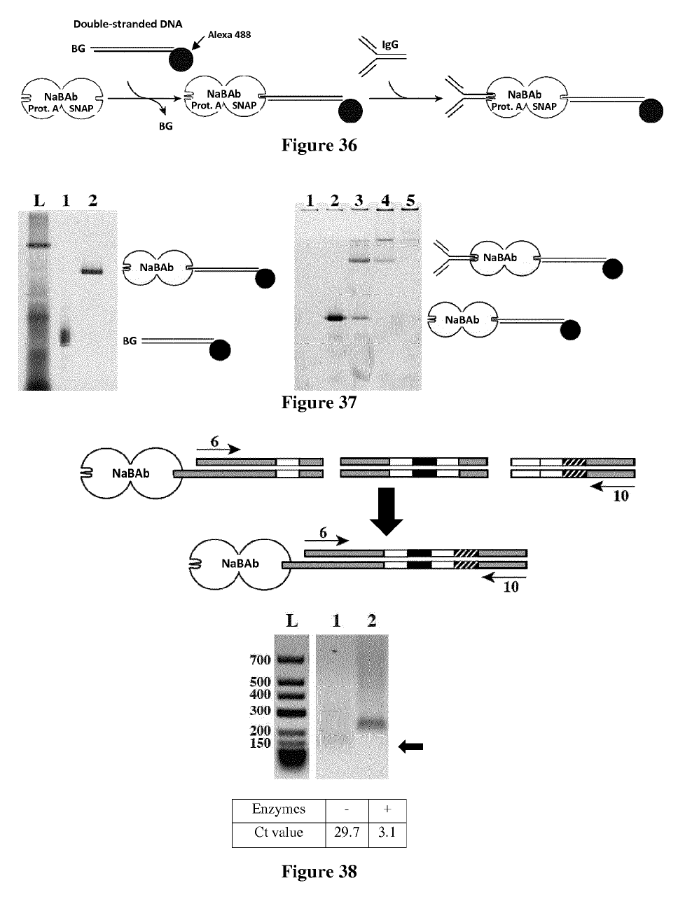

We prepared a construct in which the sequence coding for the protein A was placed in fusion with a His-tagged version of the SNAP-tag (New England Biolabs) and placed the construct on an expression plasmid. The corresponding protein was then overproduced in E. coli and purified by affinity chromatography. The SNAP domain is a catalytic module reacting with benzyl-guanine-displaying substrate molecules. Upon reaction, a single molecule of substrate is covalently and irreversibly attached to the SNAP module (FIG. 36). Moreover, this protein modified with a single DNA molecule should also be able to strongly associate with antibodies via its protein A domain. In this work, we show that our fusion protein is indeed able to be labelled by a single DNA molecule and that the resulting complex is still able to interact with antibodies.

Experimental Procedure

A template DNA (molecule 31) possessing a unique AlwNI as well as a UMI of 8 randomized positions was PCR amplified using a primer modified with a benzyl-guanine (BG) group (molecule 32) allowing for grafting the DNA on the SNAP module of NaBAb and a primer modified with an Alexa-488 fluorescent group (molecule 33) to fluorescently label the DNA. 10 μmols of template 31 were mixed with 1 nmol of each primer (32 and 33) in 1 mL of PCR solution containing 0.2 mM of each dNTP, 20 U of Q5 DNA polymerase (New England Biolabs) and the corresponding buffer at the recommended concentration. The mixture was then placed in thermocycler and subjected to an initial denaturation step of 30 sec at 98° C. followed by 25 cycles of 10 sec at 98° C., 10 sec at 55° C. and 30 sec at 72° C. Finally, the program was concluded by a final extension step of 2 min at 72° C. Amplification products were the purified using the kit Wizard SV Gel and PCR clean up system (Promega) and quantified with a Nanodrop device. 200 μmols of BG/Alexa488 dually labelled DNA were then mixed with 180 μmol of purified NaBAb in 1 mL of CutSmart® buffer (New England Biolabs) diluted at the recommended concentration (lx) and supplemented with 1 mM DTT. The same experiment in which NaBAb was omitted was performed in parallel and used as control. The mixture was incubated for an hour at 37° C. and an aliquot was analyzed on polyacrylamide gel electrophoresis and Alexa488 fluorescence revealed (FIG. 37, left panel).

In a second experiment, 27 μmols of purified NaBAb were mixed with 20 μmols of BG/Alexa488 dually labelled DNA in 40 μL of CutSmart® buffer (New England Biolabs) diluted at the recommended concentration (1×) and supplemented with 1 mM DTT. Moreover, the mixture was also supplemented with 225, 112, 62.5 μg/mL of total Human IgG (Sigma Aldrich). Reactions in which IgG and/or the dually labelled DNA were omitted were used as control. The mixture was incubated for an hour at 37° C. and an aliquot was analyzed on polyacrylamide gel electrophoresis and Alexa488 fluorescence revealed (FIG. 37, right panel).

Results & Conclusions

Incubating BG/Alexa488 dually labelled DNA with a slight excess of NaBAb allowed grafting all the DNA onto the SNAP module of NaBAb as attested by the complete up-shift of the DNA band on the gel (FIG. 37, left panel). Forming the NaBAb-DNA covalent complex in the presence of IgG showed that NaBAb-DNA/IgG ternary complex can readily form (FIG. 37, right panel, lanes 3 to 5). Moreover, considering the average molecular weight of IgG to be ˜150 kDa one can see that the 27 μmols of NaBAb-DNA complex are completely shifted in the presence of an equimolar amount of IgG (125 mg/mL or 30 μmols IgG, lane 4) whereas only half of the complex is shifted using twice less IgG. These data indicate that not only NaBAb can be covalently linked to a single DNA molecule, but mixing it stoichiometrically with an IgG allows forming a NaBAb-DNA/IgG ternary complex in which one antibody molecule is predominantly labelled with a single DNA molecule.

b. Labelling NaBAb-DNA Complex with UEIs

We investigated the possibility of labelling the NaBAb-DNA complex by UIs using the labelling strategy presented in Example 3.

Experimental Procedure

500 atto moles of template UEI-Calibrators and UEIs (molecules 11 and 15) were co-amplified in 200 μl of PCR mixture supplemented with 0.2 μM of each primer (2, 5, 10 and 12) as described in Example 3-section a. Upon thermocycling, 10 μL duplex PCR were added to 10 μL of CutSmart® buffer (New England Biolabs) diluted at the recommended concentration (1×) supplemented with 2 μmol of NaBAb-DNA complex (prepared as described in section a of this Example), 2 mM rATP, 20 mM DTT, 2.5 DraIII HF (New England Biolabs), 2.5 U/μL AlwN1 (New England Biolabs), 25 U/μL T4 DNA ligase (New England Biolabs), 10 μM coumarin acetic and 0.1% pluronic F68. The mixture was then subjected to 5 cycles of temperature: 15 min at 37° C. and 45 min at 16° C. In parallel, we performed a control reaction in which restriction/ligation enzymes were omitted. At the end of the incubation, 1 μL of each reaction was analyzed by quantitative PCR using the kit Sso-Fast Evagreen (Bio-Rad) supplemented in primers 6 and 10, and the amplification products were analyzed on a 1% agarose gel (FIG. 38).

Results & Conclusions

In this section, we showed that a DNA covalently associated to the NaBAb protein can serve as UI acceptor using or restriction/ligation strategy. Indeed, in absence of the enzymes more than 26 additional amplification cycles (delta Ct >26) were required the reach the threshold (FIG. 38, compare columns − and + enzymes), indicating that there is at least a 33 million-times less UI-target DNA formed in the absence of enzyme. Moreover, gel electrophoresis analysis (FIG. 38) showed that specific band of the expected size is obtained only in the presence of the enzymes (lane 2).

In conclusion, in this example, we demonstrated that the NaBAb fusion protein can be used to specifically label IgG with a unique DNA molecule and that, even though it is covalently attached to the NaBAb protein, this DNA can further serve as UI acceptor using our restriction/ligation strategy. Moreover, since the recombination occurs in native conditions preserving the association of non-covalent complexes (e.g. antibody/antigen), the combined use of NaBAb-DNA/IgG complex and of UI encoding can be used to label and encoded IgG-recognized targets (e.g. protein) and quantify them by Next Generation sequencing (e.g. MiSeq).

### GENERAL SUMMARY & CONCLUSIONS

The different examples reported here demonstrate the complete technical feasibility of the method of the invention. Indeed, we demonstrated that the two components (the UEI-Calibrator and the UEI) of Unique Identifiers (UIs) can be efficiently co-amplified in a duplex PCR prior to being recombined together by restriction/ligation reactions to form UIs both in bulk (Example 2.a) and emulsified reactions (Example 2.b). The pool of UI contained in droplet can then be used as signature allowing for unambiguously assigning the sequences contained in a pool of Next Generation Sequencing data to the droplet they originate from (Example 2.c). Moreover, changing the initial number of different template UEI-Calibrators and UEIs was shown to allow adjusting the complexity of the signature (Example 2.d).

In addition, we demonstrated that these UI can be further attached to a target DNA molecule displaying a 3′ overhang compatible with a restriction site present on the UI both in bulk (Example 3.a) and in droplets (Example 3.b). Again, the use UI-based signature allowed to unambiguously assign each DNA sequence to the droplet it originated with a great droplet-to-droplet reproducibility.

Moreover, we showed that RNA can be efficiently reverse transcribed into cDNA in droplet (Example 4.a) using reverse transcription primers either competent for being later coupled with an UI (Example 4.b) or conjugated to an UI prior to being used for reverse transcribing the target RNA (Example 5). In both cases, similar yield of RT and UI coupling were obtained. Our encoding strategy is not limited to nucleic acids since we showed that our UI grafting strategy can also be applied to IgG labeled with a single DNA molecule possessing a restriction site making it compatible with our restriction/ligation approach (Example 6). This last scenario makes possible to encode non-nucleic acids targets such as proteins in a way that preserve non-covalent biological complexes (e.g. antibody/antigen complexes).

Finally, we showed that cells can be isolated and lysed into small 4 pL droplets (Example 1). Not only the material can be released from the cells, but the size of the droplets makes the directly compatible with the use of the droplet fusion device (FIG. 33). Therefore, cell extract can be directly supplemented with the reagents allowing for reverse transcribing target RNAs and/or antibodies coupled to a single DNA molecule. Upon reverse transcription/antigen capture, a different set of UEI-Calibrators/UEIs can then be delivered to each droplet together with the restriction/ligation enzymes using the Barcoding chip (FIG. 27) validated in Example 3. Finally, upon indexing and next generation sequencing the use of our bioinformatics algorithm (FIG. 29) allows to reassign each sequence (initially corresponding to an RNA, a protein or another molecule) to the droplet it originates from and this way allows for quantifying the content of each cell.

### REFERENCES

Benito, Y., et al. RNA, 2000. 6(5): p. 668-79.
Brown, R. B. & Audet, J. R. Soc. Interface 5, S131-S138 (2008)
Islam et al. Nat. Protoc. 2012; 7:813-828.
Kintses et al. Chem. Biol. 2012. 19, 1001-1009
Klein et al., Cell. 2015. 161, 1187-1201
Macosko et al. Cell. 2015, 161, 1202-1214
Mazutis, L., et al., Analytical Chemistry, 2009. 81(12): p. 4813-21.
Novak et al. Angew. Chem. Int. Ed. 50, 390-395 (2011)
Rau et al. Appl. Phys. Lett. 2004. 84, 2940-2942
Rotem et al., PLoS ONE, 2015, 10(5): e0116328
Ryckelynck, M., et al., RNA, 2015. 21(3): p. 458-69.
Siegel, A. C., et al., Advanced Materials, 2007. 19(5): p. 727-733.
Streets et al. Proc. Natl. Acad. Sci. USA, 2014, 111, 7048-7053
Xia, Y. N. and G. M. Whitesides, Soft lithography. Angewandte Chemie-International Edition, 1998. 37(5): p. 551-575.

1-18. (canceled)

19. A method of labelling a plurality of molecular targets from a plurality of entities while preserving the integrity of the single-entity information, said method comprising:
providing a first set of emulsion droplets comprising droplets containing labelled molecular targets, wherein each of these droplets contains a plurality of molecular targets originating from no more than one entity and wherein, in each of these droplets, each molecular target is labelled with a molecular identification DNA sequence comprising (i) a unique molecular identification (UMI) barcode which is different for each molecular target and (ii) an overhang or an overhang producing restriction site;
providing a second set of emulsion droplets comprising droplets containing entity identification sequences, wherein each of these droplets contains at least one entity identification sequence which is a DNA sequence comprising a unique entity identification (UEI) barcode which is different for each droplet of the second set, and a an overhang producing restriction site;
fusing droplets of the first set with droplets of the second set wherein a droplet of the first set is fused with no more than one droplet of the second set; and
ligating UEI barcodes to labelled molecular targets, optionally after restriction enzyme digestion, and
optionally breaking the emulsion.

20. The method of claim 19, wherein the method further comprises:
encapsulating a plurality of entities within emulsion droplets, each droplet containing no more than one entity, and optionally lysing said entities within the droplets to release molecular targets;
labelling said molecular targets with probes, each probe comprising:
a capture moiety capable of specific binding or ligation to a molecular target or to an adaptor linked to said molecular target, and
a DNA moiety comprising (i) a region proximal to the capture moiety and comprising the unique molecular identification (UMI) sequence and (ii) a region distal from the capture moiety and comprising an overhang or an overhang producing restriction site,
thereby obtaining the first set of emulsion droplets.

21. The method of claim 19, wherein the method further comprises encapsulating a plurality of entity identification sequences within emulsion droplets, each droplet containing no more than one entity identification sequence, with an amplification reaction mixture, and
amplifying the entity identification sequences within droplets;
thereby obtaining the second set of emulsion droplets.

22. The method of claim 19, wherein the method further comprises:
encapsulating a plurality of entity identification sequences within emulsion droplets in the presence of UEI-calibrators, wherein at least some droplets comprise one or several entity identification sequences and one or several UEI-calibrators, and wherein said UEI-calibrators are DNA sequences comprising a unique calibrator barcode which is different for each UEI-calibrator and for each droplet, and one or two overhang producing restriction sites; and
amplifying entity identification sequences and/or UEI-calibrators within droplets;
thereby obtaining the second set of emulsion droplets.

23. The method of claim 22, wherein, after fusion of droplets of the first and second sets, (i) UEI calibrator barcodes and UEI barcodes and (ii) UEI barcodes and labelled molecular targets are assembled through restriction enzyme digestion and ligation of compatible overhangs of (i) UEI calibrators and entity identification sequences and of (ii) entity identification sequences and labelled molecular targets.

24. The method of claim 22, wherein, after fusion of droplets of the first and second sets, UEI calibrator barcodes, UEI barcodes and labelled molecular targets are assembled through restriction enzyme digestion and ligation of compatible overhangs of (i) UEI calibrators and labelled molecular targets and (ii) UEI calibrators and entity identification sequences, or of i) UEI calibrators and entity identification sequences and (ii) entity identification sequences and labelled molecular targets.

25. The method of claim 22, wherein entity identification sequences and UEI-calibrators are assembled through their compatible overhangs before amplification and, after fusion of droplets of the first and second sets, the amplified fragment comprising UEI calibrator and UEI barcodes is ligated to labelled molecular targets through compatible overhangs of i) UEI calibrators and labelled molecular targets or (ii) entity identification sequences and labelled molecular targets.

26. The method of claim 19, wherein at least some of molecular targets are nucleic acids and at least some probes comprise:
a capture moiety which is a single stranded DNA region which drives the specific recognition of a nucleic acid molecular target through conventional Watson-Crick base-pairing interactions; and
a DNA moiety comprising a 3′ single stranded region comprising the unique molecular identification (UMI) sequence and a 5′double-stranded region comprising the overhang or overhang producing restriction site.

27. The method of claim 26, wherein said nucleic acid molecular targets are labelled using said probes as priming sites for a DNA polymerase synthesizing complementary strands of molecular targets.

28. The method of claim 26, wherein at least some of molecular targets are RNA molecules and the DNA polymerase is a reverse transcriptase.

29. The method of claim 20, wherein at least some probes comprise a capture moiety which is:
(i) a binding moiety that specifically binds to a molecular target and is directly bound to the DNA moiety;
(ii) a chimeric protein comprising a first domain that specifically binds to a molecular target and a second domain that binds to the DNA moiety; or
(iii) a binding moiety that binds specifically to a molecular target and a protein bridge, said protein bridge comprising a first domain that binds to the binding moiety and a second domain that binds to the DNA moiety.

30. The method of claim 29, wherein (i) the binding moiety or the first domain of the chimeric protein is selected from the group consisting of an antibody, a ligand of a ligand/anti-ligand couple, a peptide aptamer, a nucleic acid aptamer, a protein tag, or a chemical probe (e.g. suicide substrate, activity-based probes ABP) reacting specifically with a molecular target or a class of molecular targets, (ii) the first domain of the protein bridge is an immunoglobulin-binding bacterial protein, and/or (iii) the second domain of the protein bridge or the chimeric protein is selected from the group consisting of SNAP-tag, CLIP-tag or Halo-Tag.

31. The method of claim 20, wherein at least some probes comprise a capture moiety comprising an antibody moiety specific to a molecular target and a protein bridge, said protein bridge comprising a first domain that binds to a Fc region of the antibody moiety and a second domain that binds to the DNA moiety.

32. The method of claim 19, wherein the entity is a cell, or a particle or an emulsion droplet.

33. The method of claim 19, wherein the entity is a particle or an emulsion droplet exposing molecular targets on its outer surface, and the method further comprises:
labelling said molecular targets with probes, each probe comprising:
a capture moiety capable of specific binding or ligation to a molecular target or to an adaptor linked to said molecular target; and
a DNA moiety comprising (i) a region proximal to the capture moiety and comprising the unique molecular identification (UMI) sequence and (ii) a region distal from the capture moiety and comprising an overhang or an overhang producing restriction site; and
encapsulating entities attached to labelled molecular targets within emulsion droplets, each droplet containing no more than one entity,
thereby obtaining the first set of emulsion droplets.

34. A method of quantifying one or several molecular targets from a plurality of entities with single-entity resolution, said method comprising:
labelling said molecular targets according to the method of claim 19;
capturing said labelled molecular targets,
amplifying sequences comprising UMI and UEI barcodes, and optionally UEI-calibrator barcodes;
sequencing amplified sequences.

35. A kit comprising:
a microfluidic device; and/or
one or several probes comprising a capture moiety capable of specific binding or ligation to a molecular target or to an adaptor linked to said molecular target, and a DNA moiety comprising (i) a region proximal to the capture moiety and comprising the unique molecular identification (UMI) sequence and (ii) a region distal from the capture moiety and comprising an overhang or an overhang producing restriction site; and/or
one or several entity identification sequences comprising DNA sequences comprising a unique entity identification (UEI) barcode and an overhang producing restriction site; and/or
one or several UEI-calibrators comprising DNA sequences comprising a unique calibrator barcode which is different for each UEI-calibrator and one or two overhang producing restriction sites; and/or
one or several primers suitable to amplify entity identification sequences and/or UEI-calibrators; and/or
an aqueous phase and/or an oil phase; and optionally
a leaflet providing guidelines to use such a kit.

36. The kit of claim 35, wherein the microfluidic device comprises:
a first emulsion re-injection module or on-chip droplet generation module;
a second emulsion re-injection module or on-chip droplet generation module;
a droplet-pairing module; and
a module coupling droplet fusion to injection,
wherein emulsion re-injection modules and/or on-chip droplet generation modules are in fluid communication and upstream to the droplet-pairing module, the droplet-pairing module is in fluid communication and upstream to the module coupling droplet fusion to injection.
